SEQUENCE LISTING (43 sequences)

SEQ ID NO: 1 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 441 aa, synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 441 aa, synthetic construct; identical to SEQ ID NO: 2
  MOD_RES 224: note = Lactylated lysine
  MOD_RES 225: note = Lactylated lysine
  MOD_RES 257: note = Lactylated lysine
  MOD_RES 267: note = Lactylated lysine
  MOD_RES 281: note = Lactylated lysine
  MOD_RES 311: note = Lactylated lysine
  MOD_RES 317: note = Lactylated lysine
  MOD_RES 321: note = Lactylated lysine
  MOD_RES 331: note = Lactylated lysine

SEQ ID NO: 4 - protein, 16 aa, synthetic construct
  MOD_RES 10: note = Lactylated lysine
  MOD_RES 11: note = Lactylated lysine
CPTPPTREPKKVAVVR

SEQ ID NO: 5 - protein, 14 aa, synthetic construct
  MOD_RES 7: note = Lactylated lysine
  MOD_RES 8: note = Lactylated lysine
PPTREPKKVAVVRC

SEQ ID NO: 6 - protein, 16 aa, synthetic construct
CPTPPTREPKKVAVVR

SEQ ID NO: 7 - protein, 12 aa, synthetic construct
  MOD_RES 7: note = Lactylated lysine
CQIINKKLDLSN

SEQ ID NO: 8 - protein, 15 aa, synthetic construct
  MOD_RES 5: note = Lactylated lysine
CINKKLDLSNVQSKC

SEQ ID NO: 9 - protein, 12 aa, synthetic construct
CQIINKKLDLSN

SEQ ID NO: 10 - protein, 14 aa, synthetic construct
  MOD_RES 6: note = Lactylated lysine
GNIHHKPGGGQVEC

SEQ ID NO: 11 - protein, 12 aa, synthetic construct
  MOD_RES 6: note = Lactylated lysine
GNIHHKPGGGQC

SEQ ID NO: 12 - protein, 14 aa, synthetic construct
GNIHHKPGGGQVEC

SEQ ID NO: 13 - protein, 16 aa, synthetic construct
  MOD_RES 10: note = Lactylated lysine
  MOD_RES 11: note = Lactylated lysine
CTHVPGGGNKKIETHK

SEQ ID NO: 14 - protein, 14 aa, synthetic construct
  MOD_RES 8: note = Lactylated lysine
  MOD_RES 9: note = Lactylated lysine
CVPGGGNKKIETHK

SEQ ID NO: 15 - protein, 16 aa, synthetic construct
CTHVPGGGNKKIETHK

SEQ ID NO: 16 - protein, 9 aa, synthetic construct
LRKLRKRLL

SEQ ID NO: 17 - protein, 39 aa, synthetic construct
SSVIDALQYKLEGTTRLTRKRGLKLATALSLSNKFVEGS

SEQ ID NO: 18 - protein, 10 aa, synthetic construct
LRKLRKRLLR

SEQ ID NO: 19 - protein, 29 aa, synthetic construct
YTIWMPENPRPGTPCDIFTNSRGKRASNG

SEQ ID NO: 20 - protein, 15 aa, synthetic construct
GGGGYGRKKRRQRRR

SEQ ID NO: 21 - protein, 7 aa, synthetic construct
AGILKRW

SEQ ID NO: 22 - protein, 18 aa, synthetic construct
CNCKAPETALCARRCQQH

SEQ ID NO: 23 - protein, 9 aa, synthetic construct
  SITE 1: note = X = 2,3-diaminopropionic acid
XKAPETALD

SEQ ID NO: 24 - protein, 12 aa, synthetic construct
PWVPSWMPPRHT

SEQ ID NO: 25 - protein, 12 aa, synthetic construct
TGNYKALHPHNG

SEQ ID NO: 26 - protein, 12 aa, synthetic construct
THRPPMWSPVWP

SEQ ID NO: 27 - protein, 31 aa, synthetic construct
PWVPSWMPPRHTPWVPSWMPPRHTKKGKCFG

SEQ ID NO: 28 - protein, 5 aa, synthetic construct
HAYED

SEQ ID NO: 29 - protein, 9 aa, synthetic construct
CNSRLHLRC

SEQ ID NO: 30 - protein, 9 aa, synthetic construct
CENWWGDVC

SEQ ID NO: 31 - protein, 20 aa, synthetic construct
WRCVLREGPAGGCAWFNRHL

SEQ ID NO: 32 - protein, 441 aa, synthetic construct; identical to SEQ ID NO: 2
  MOD_RES 32: note = Lactylated lysine

SEQ ID NO: 33 - protein, 441 aa, synthetic construct; identical to SEQ ID NO: 2
  MOD_RES 225: note = Lactylated lysine

SEQ ID NO: 34 - protein, 441 aa, synthetic construct; identical to SEQ ID NO: 2
  MOD_RES 257: note = Lactylated lysine

SEQ ID NO: 35 - protein, 441 aa, synthetic construct; identical to SEQ ID NO: 2
  MOD_RES 267: note = Lactylated lysine

SEQ ID NO: 36 - protein, 441 aa, synthetic construct; identical to SEQ ID NO: 2
  MOD_RES 311: note = Lactylated lysine

SEQ ID NO: 37 - protein, 441 aa, synthetic construct; identical to SEQ ID NO: 2
  MOD_RES 317: note = Lactylated lysine

SEQ ID NO: 38 - protein, 441 aa, synthetic construct; identical to SEQ ID NO: 2
  MOD_RES 331: note = Lactylated lysine

SEQ ID NO: 39 - protein, 441 aa, synthetic construct; identical to SEQ ID NO: 2
  MOD_RES 321: note = Lactylated lysine

SEQ ID NO: 40 - protein, 441 aa, synthetic construct; identical to SEQ ID NO: 2
  MOD_RES 281: note = Lactylated lysine

SEQ ID NO: 41 - protein, 18 aa, synthetic construct
  MOD_RES 10: note = Lactylated lysine
CGSLGNIHHKPGGQVEVK

SEQ ID NO: 42 - protein, 17 aa, synthetic construct
  MOD_RES 16: note = Lactylated lysine
IGSLDNITHVPGGGNKK

SEQ ID NO: 43 - protein, 17 aa, synthetic construct
  MOD_RES 17: note = Lactylated lysine
IGSLDNITHVPGGGNKK

(19) United States
(12) Patent Application Publication
(10) Pub. No.: US 2026/0186004 A1
(43) Pub. Date: Jul. 2, 2026

(54) COMPOSITIONS AND METHODS FOR DETECTION AND TREATMENT OF TAUOPATHIES

(71) Applicant: University of Kansas, Lawrence, KS (US)

(72) Inventors: Ning Wang, Lawrence, KS (US); Xiaoyu Zhang, Roeland Park, KS (US)

(73) Assignee: University of Kansas, Lawrence, KS (US)

(21) Appl. No.: 19/425,491

(22) Filed: Dec. 18, 2025

Related U.S. Application Data
(60) Provisional application No. 63/738,871, filed on Dec. 26, 2024.

(51) Int. Cl. G01N 33/68

(52) U.S. Cl. CPC G01N 33/6896; G01N 2440/00; G01N 2800/2821

(57) ABSTRACT

It has been established that increased levels of lactylated of tau protein in the brain and CNS is associated with tauopathies and neurodegenerative diseases, and increased levels of lactylated lysine on tau protein has been established as a marked for AD. Compositions and methods for the detection and treatments of tauopathies have been developed. In some forms, the methods identify and quantify lactylated lysine on tau protein in a biological sample from a subject to diagnose a tauopathy, such as AD. In some forms, the methods identify a subject as having or at risk of having a tauopathy. In some forms, the methods include treating the tauopathy. Compositions to identify a tauopathy include lactylated tau binders, such as antibodies are also described. Compositions to treat or prevent a tauopathy, such as lactylated tau peptides having a defined lactyllysine, are also described.

### CROSS-REFERENCE TO RELATED APPLICATIONS

This application claims the benefit of and priority to U.S. Application No. 63/738,871 filed Dec. 26, 2024, the contents of which are incorporated by reference herein in their entirety.

### STATEMENT REGARDING FEDERALLY SPONSORED RESEARCH

This invention was made with government support under HD103888 and AG072973 awarded by the National Institutes of Health. The Government has certain rights in the invention.

### REFERENCE TO THE SEQUENCE LISTING

The Sequence Listing submitted as an XML file named “KU25-036M-02US_ST26.xml” created on Dec. 18, 2025, and having a size of 56,748 bytes is hereby incorporated by reference pursuant to 37 C.F.R. § 1.834(c)(1).

### FIELD OF THE INVENTION

The invention is generally related to the field of detecting and preventing neurodegenerative diseases, and more particularly to is general compositions and methods for the detection, reduction and prevention of lactylation of tau proteins in the human brain.

### BACKGROUND OF THE INVENTION

A key feature in Alzheimer's disease (AD) pathogenesis is metabolic dysfunction [1]. Originally observed in AD cells, the Warburg effect [2], has received increasing attention. This effect entails a preferential shift towards aerobic glycolysis over oxidative phosphorylation, even in the presence of oxygen. As such, most glucose is converted to lactate, which is then supplied to neurons as an alternative fuel in AD [3, 4].

In addition to its metabolic function, lactate has been shown to induce a post-translational modification (PTM) on histones and proteins known as “lactylation”, which is the addition of lactyl group to lysine (K) residues [5]. Lactylation of lysine residues in histones functions as an epigenetic modification to regulate gene transcription [5]. In the context of AD, it has been reported that elevated histone H4 lysine 12 lactylation (H4K12la) levels in microglia near β-amyloid (AB) plaques enhance glycolytic gene transcription, which worsens microglial dysfunction in AD [6]. More recently, protein lactylation on lysine residues has been detected, and protein lactylation as a PTM has been shown to play important roles in regulating their biological functions [7]. However, whether protein lactylation is implicated in AD remains unclear [8].

Amyloid plaques and neurofibrillary tangles (NFTs) are neuropathological hallmarks of AD. While amyloid plaques are composed of β-amyloid (AB) peptides, NFTs are composed of intracellular aggregates of hyperphosphorylated microtubule binding protein tau (tauopathy). Abnormal accumulation of hyperphosphorylated tau as insoluble NFTs is implicated in the pathogenesis of AD as well as other neurodegenerative disorders involving tauopathy. Of the two pathological features, amyloid plaques and NFTs, the presence of NFTs strongly correlates with symptoms such as cognitive decline in AD patients. Notably, tau is profoundly regulated by post-translational modifications (PTMs), e.g., phosphorylation, ubiquitination, acetylation, and is profoundly implicated in tauopathy.

Thus, there remains a need for improved means of diagnosing and treating tauopathies, such as lactylation of tau protein in the brain of patients with Alzheimer's disease.

It is an object of the invention to provide compositions and methods thereof for detecting lactylation of tau protein in a subject.

It is another object of the invention to provide compositions and methods thereof for diagnosing tauopathies, such as lactylation of tau protein in the brain of patients with Alzheimer's disease.

It is another object of the invention to provide compositions and methods thereof for treating and preventing tauopathies.

It is another object of the invention to provide compositions and methods thereof for treating and preventing Alzheimer's disease in patients in need thereof.

### SUMMARY OF THE INVENTION

It has been established that lactylation of tau protein in the brain and central nervous system (CNS) contributes to and results from neurodegenerative diseases such as Alzheimer's disease. Detection of lactylated tau protein correlates with severity and progression of AD in a subject. Therefore, compositions and methods for the detection and quantitation of lactylated tau are provided. It has also been established that disruption of the processes that give rise to lactylation of tau protein and/or reduction in the amount of lactylated tau protein reduces or prevents the progression and severity of neurodegenerative diseases such as AD. Therefore, compositions and methods for the reduction and/or prevention of the lactylation of tau protein in the brain are also provided. In some forms, the reduction and/or prevention of the lactylation of tau protein treats or prevents one or more symptoms of a neurodegenerative disease or disorder.

Methods of detecting or quantifying lactylation of tau protein, including detecting lactylated tau protein in a sample including tau protein are described. Any suitable detection method can be used. Options include, but are not limited to, immunoassays and other antibody-based techniques, mass spectrometry, chemical labeling and imaging, chromatographic methods, animal behavior based assays, and biosensor analyses. In some forms, the detecting includes contacting the sample with one or more binding moieties that selectively bind lactylated tau protein. In some forms, the tau protein is within a biological sample from a subject, optionally wherein the biological sample includes cells or a cell lysate or a fraction thereof, optionally wherein the cells or cell lysate are obtained as a biopsy from the subject.

In some forms, the biological sample includes a bodily fluid selected from the group including cerebral spinal fluid (CSF), blood, plasma, urine, saliva, nasal fluid, bone marrow, brain tissue, vomit, and feces. In some forms, the biological sample includes CSF. In some forms, the methods further include determining that the biological sample includes lactylated tau protein if the level detected is higher in the biological sample than in a control. In some forms, the control includes a recombinant non-lactylated tau protein, or a biological sample including a tau protein from a healthy subject. In some forms, binding is detected by an assay selected from an immunoassay, immunohistochemistry, Western blotting, surface plasmon resonance, flow cytometry (FACS) analysis, and biochip analyses. Exemplary immunoassays include an enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, and immuno-chromatography assays. In some forms, the one or more binding moieties do not bind to non-lactylated tau protein, optionally wherein the one or more binding moieties binds to lactylated lysine (Kla). In some forms, the one or more binding moieties is selected from a small molecule, a protein, and a nucleic acid. In some forms, the one or more binding moieties is or includes an antibody or an antigen binding fragment thereof. In some forms, the antibody or antigen binding fragment thereof includes an antigen binding domain that immuno-specifically binds to one or more of CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIHH(K-lactyl)PGGGQVEC (SEQ ID NO:10); GNIHH(K-lactyl)PGGGQC (SEQ ID NO:11); CTHVPGGGN(K-lactyl)(K-lactyl)IETHK (SEQID NO:13); or CVPGGGN(K-lactyl)(K-lactyl)IETHK (SEQ ID NO:14).

Methods of diagnosing a subject with a disease or disorder associated with lactylated tau protein, including detecting or quantifying lactylated tau protein within a sample from the subject are also provided. In some forms, the disease or disorder associated with lactylated tau protein is selected from Alzheimer's disease (AD), Argyrophilic grain disease (AGD), Chronic Traumatic Encephalopathy (CTE), Dementia pugilistica (chronic traumatic encephalopathy), frontotemporal dementia, frontotemporal lobar degeneration, gangliocytoma, Ganglioglioma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis,tangle-predominant dementia, lead encephalopathy, tuberous sclerosis, Hallervorden-Spatz disease, lipofuscinosis, and corticobasal degeneration. In certain forms, the disease or disorder is Alzheimer's disease. In some forms, the methods further include treating the subject. For example, in some forms, the methods include administering a therapy effective for treating diseases and disorders characterized by the presence of lactylated tau protein. In some forms, the treatment includes administering to the subject an effective amount of a lactylated tau protein or fragment thereof, to reduce or alleviate one or more symptoms of the disease or disorder. In some forms, the lactylated tau protein or fragment thereof is administered to the subject at dosage of between 0.01 mg/kg and about 1.0 mg/kg, inclusive. In some forms, the lactylated tau protein or fragment thereof is administered to the subject every day, every two days, every three days, every four days, every five days, every six days, every seven days, once every two weeks, or once a month. In some forms, the lactylated tau protein is selected from the group including CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIHH(K-lactyl)PGGGQVEC (SEQ ID NO:10); GNIHH(K-lactyl)PGGGQC (SEQ ID NO:11); CTHVPGGGN(K-lactyl)(K-lactyl)IETHK (SEQ ID NO:13); and CVPGGGN(K-lactyl)(K-lactyl)IETHK (SEQ ID NO:14). In some forms, the lactylated tau protein is selected from the group including CQIINK(K-lactyl)LDLSN (SEQ ID NO:7) and CINK(K-lactyl)LDLSNVQSKC (SEQ ID NO:8). In some forms, the lactylated tau protein is CQIINK(K-lactyl)LDLSN (SEQ ID NO:7).

Compositions including an isolated tau protein, or a fragment thereof, wherein the tau protein, or a fragment thereof includes lactylation of one or more lysine residues are also provided. In some forms, the tau protein includes an amino acid sequence of any one of SEQ ID NO:2, wherein one or more lysine resides is lactylated, wherein the one or more lactylated lysine residue is selected from the group including K224, K225, K257, K267, K281, K311, K317, K331, and K321. In some forms, the tau protein includes an amino acid sequence of any one of SEQ ID NOs:3, or 32-40. In some forms, the tau protein includes an amino acid sequence of SEQ ID NO:36. In some forms, the tau protein or fragment thereof is in an amount effective for use as a positive control composition for an assay for diagnosing a subject with a disease or disorder associated with lactylated tau protein.

A kit including an isolated tau protein, or a fragment thereof, wherein the tau protein, or a fragment thereof includes lactylation of one or more lysine residues selected from K224, K225, K257, K267, K281, K311, K317, K331, and K321 of SEQ ID NO:2 is also described.

An antibody that selectively binds lactylated tau protein, or an antigen binding fragment thereof, wherein the antibody is prepared according to a method including: (a) immunizing a subject with the recombinant tau protein; and (b) isolating an antibody that immuno-specifically binds to the recombinant tau protein from the subject is also provided.

In some forms, the subject is immunized with one or more of SEQ ID NOs:3, 4-5, 7-8, 10-11, 13-14, or 32-40. In some forms, the antibody is an intact antibody or functional antibody fragment or fusion protein. In some forms, the functional fragment or fusion protein is selected from Fab fragments, F(ab′)2 fragments, Fab′ fragments, Fv fragments, recombinant IgG (rlgG) fragments, single chain antibody fragments optionally single chain variable fragments (scFv), and single domain antibodies optionally selected from sdAb, sdFv, and nanobody fragments. In some forms, the antibody is selected from intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv. In some forms, the antibody is an IgM, IgE, IgA, IgD, or IgG optionally an IgG₁, IgG₂, IgG₃, or IgG₄.

### BRIEF DESCRIPTION OF THE DRAWINGS

FIGS. 1A-1J show elevated lactate signature and tau lactylation in AD samples. FIGS. 1A-1B are graphs of the association of lactate signature levels with phosphorylated tau (p-tau) (FIG. 1A) and total tau (FIG. 1A) levels from CSF proteomics datasets (n=19 control and 20 AD samples). Protein levels were detected by ELISA. FIG. 1C is a graph of immunofluorescence staining using anti-lactylated lysine (Kla) and anti-phosphorylated tau (AT8) antibodies on the brain's frontal cortex from postmortem human control and AD subjects, showing correlation of AT8 staining intensities with Kla staining intensities in individual cells. FIGS. 1D-1I are violin plots showing relative levels of proteins Kla(IP) (FIG. 1D), tau5(IP) (FIG. 1E), AT8 (FIG. 1F), tau368N (FIG. 1G), tau5(IP) (FIG. 1H), and Kla(IP) (FIG. 1I), measured by immunoblot analysis. Lactylated and total tau levels were determined by immunoprecipitation/immunoblot analysis using anti-lactylated lysine (Kla) and tau5 antibodies, respectively, in 6 human control and 6 AD samples. Protein lysates were immunoblotted using phosphorylated tau (AT8), cleaved tau (tau368N), tau5, and anti-lactylated lysine (Kla) antibodies. FIG. 1J is a graphical correlation of the levels of phosphorylated tau (AT8), lactylated lysine (Kla), and cleaved tau (Tau368N) with lactylated tau in human control and AD samples measured by Western blot analysis.

FIGS. 2A-2F show quantitative proteomics analysis of tau lactylation in human control and AD brain samples. FIG. 2A is a schematic showing tau-441 and its key domains, with six lysine sites that were identified to be lactylated by MS: K257, K267, K311, K317, K331, and possibly K321. FIG. 2B is a mass spectrogram showing identification of tau K331 lactylation, as set forth in the amino acid sequence CGSLGNIHH(K-lactyl)PGGQVEVK (SEQ ID NO:41). FIG. 2C is a bar graph showing percentages of each of human control and AD brain samples with or without tau K331 lactylation (n=38 control and 47 AD samples). FIGS. 2D-2F are violin plots showing Log2-normalized abundance of tau K331 lactylation in human control versus AD brain samples (n=31 control samples and 44 AD samples; tau K331 lactylation was not detected in 7 control and 3 AD samples) (FIG. 2D); Log2-normalized abundance of tau K331 lactylation in human control (Braak stages I [n=6], II [n=5], and III [n=2]) and AD (Braak stages IV [n=14] and V [n=26]) brain samples (FIG. 2E); and Log2-normalized abundance of tau K331 lactylation in female (n=16 control and 28 AD) versus male (n=22 control and 19 AD) brain samples (FIG. 2E), respectively.

FIGS. 3A-3U are graphs and schematics demonstrating lactate induction of lactylation of tau. FIGS. 3A-3I are violin plots showing lactylated and total tau levels, determined by immunoprecipitation/immunoblot analysis using anti-lactylated lysine (Kla) and tau5 antibodies, respectively, in HEK 293T cells treated with vehicle or lactate at 2 mM for 24 hours. Protein lysates from the soluble and insoluble fractions were immunoblotted using phosphorylated tau (AT8), cleaved tau (tau368N), tau5, and anti-lactylated lysine (Kla) antibodies. Graphs depict levels of Kla(IP) (FIG. 3A); Soluble (FIG. 3B) and Insoluble (FIG. 3C) AT8; tau5(IP) (FIG. 3D); Soluble (FIG. 3E) and Insoluble (FIG. 3F) tau368N; Soluble Kla (FIG. 3G); and Soluble (FIG. 3H) and Insoluble (FIG. 3I) tau5, respectively. FIG. 3J is a violin plot showing the flow cytometry analysis of lactate levels in tau-transfected HEK 293T cells treated with vehicle or lactate using lactate probe (SCOTfluor). FIGS. 3K-3S are violin plots showing levels of lactylated and total tau, determined by immunoprecipitation/immunoblot analysis using anti-lactylated lysine (Kla) and tau5 antibodies, respectively, in HEK 293T cells transfected with control and shRNA against LDHA. Protein lysates from the soluble and insoluble fractions were immunoblotted using phosphorylated tau (AT8), cleaved tau (tau368N), tau5, and anti-lactylated lysine (Kla) antibodies. Graphs depict levels of Kla(IP) (FIG. 3K); Soluble (FIG. 3L) and Insoluble (FIG. 3M) AT8; tau5(IP) (FIG. 3N); Soluble (FIG. 3O) and Insoluble (FIG. 3P) tau368N; Soluble Kla (FIG. 3Q); and Soluble (FIG. 3R) and Insoluble (FIG. 3S) tau5, respectively. FIG. 3T is a violin plot showing flow cytometry analysis of lactate level in HEK 293T cells transfected with control and shRNA against LDHA using lactate probe (SCOTfluor). FIG. 3U is a schematic of the regulation of glycolysis and lactate production by diverse metabolic modulators.

FIGS. 4A-4J illustrate lactylation of tau by p300 in vitro. FIG. 4A is an image of a Western blot of whole-cell extracts of 293T cells transfected with tau (tauWT-GFP) and p300-HA, collected for immunoprecipitation with the HA antibody, followed by immunoblot analysis by antibodies for total tau (tau5) and GFP, respectively. FIG. 4B is an image of a Western blot of purified tau (tauWT-His) incubated with Flag-p300, followed by Flag pull-down assay and immunoblot with anti-His tag antibody. FIG. 4C is an image of a Western blot of an in vitro lactylation assay, where purified tau (tauWT-His) was incubated with recombinant Flag-p300 with or without lactyl-CoA, followed by immunoblot analysis using antibodies against anti-lactylated lysine (Kla), anti-acetylated lysine (Kac), Flag-tag (Flag), His-tag (His), and phosphorylated tau (AT-8). FIG. 4D is an image of a Western blot of an in vitro acetylation assay, where purified tau (tauWT-His) was incubated with recombinant Flag-p300 with or without acetyl-CoA, followed by immunoblot analysis using each of antibodies against anti-lactylated lysine (Kla), anti-acetylated lysine (Kac), Flag-tag (Flag), His-tag (His), and phosphorylated tau (AT-8), respectively. FIG. 4E is a bar graph of peptide spectrum matches (PSM) of the in vitro lactylated tau assay depicted in FIG. 4C.

FIG. 4F is a schematic showing tau-441 and its key domains, indicating relative positions of each of eight lysine sites that were identified to be lactylated by MS, including K24, K67, K163, K224, K225, K331, K369, and possibly K370. FIGS. 4G-4I are mass spectrograms, showing identification of tau K331 lactylation via the peptide CGSLGNIHH(K-lactyl)PGGQVEVK (SEQ ID NO:41) (FIG. 4G); tau K369 lactylation via the peptide IGSLDNITHVPGGGN(K-lactyl)K (SEQ ID NO:42) (FIG. 411); and tau K370 lactylation via the peptide IGSLDNITHVPGGGNK(K-lactyl) (SEQ ID NO:43) (FIG. 4I), respectively, by MS. FIG. 4J is an image of a Western blot of lactylated and total tau levels, determined by immunoprecipitation/immunoblot analysis using anti-lactylated lysine (Kla) and tau5 antibodies, respectively, in HEK 293T cells transfected with wild-type tau (tauWT) and mutant tau (tau3KR) treated with vehicle or lactate at 2 mM for 24 hours. tau3KR harbors K331/369/370R triple mutation. Protein lysates from the soluble and insoluble fractions were immunoblotted using phosphorylated tau (AT8), cleaved tau (tau368N), tau5, and actin antibodies.

FIGS. 5A-5R show data to assess whether lactylation regulates tau turnover. FIGS. 5A-5F are graphs depicting relative levels of Soluble (FIG. 5A) and Insoluble (FIG. 5B) AT8; Soluble (FIG. 5C) and Insoluble (FIG. 5D) tau368N; and Soluble (FIG. 5E) and Insoluble (FIG. 5F) tau5, respectively, for each of vehicle only (Veh) and lactate, respectively, from immunoblot analysis by AT8 and tau368N antibodies, respectively, of protein lysates from the soluble and insoluble fractions, respectively, of HEK 293T cells transfected with tauWT and treated with cycloheximide (CHX). FIGS. 5G-5L are graphs depicting relative levels of Soluble (FIG. 5G) and Insoluble (FIG. 511) AT8; Soluble (FIG. 51) and Insoluble (FIG. 5J) tau368N; and Soluble (FIG. 5K) and Insoluble (FIG. 5L) tau5, respectively, for each of vehicle only (Veh) and lactate, respectively, from immunoblot analysis by AT8 and tau368N antibodies, respectively, of protein lysates from the soluble and insoluble fractions, respectively, of HEK 293T cells transfected with control and shRNA against LDHA and treated with cycloheximide (CHX). FIGS. 5M-5R are graphs depicting relative levels of Soluble (FIG. 5M) and Insoluble (FIG. 5N) AT8; Soluble (FIG. 5O) and Insoluble (FIG. 5P) tau368N; and Soluble (FIG. 5Q) and Insoluble (FIG. 5R) tau5, respectively, for each of vehicle only (Veh) and lactate, respectively, from immunoblot analysis by AT8 and tau368N antibodies, respectively, of protein lysates from the soluble and insoluble fractions, respectively, of HEK 293T cells transfected with wild-type tau (tauWT) and mutant tau (tau3KR) and treated with cycloheximide (CHX).

FIGS. 6A-6H show data to determine whether lactylation regulates tau ubiquitination and cleavage. FIG. 6A is an image of a Western blot of whole-cell extracts of HEK 293T cells transfected with tauWT-GFP and HA-tagged ubiquitin (Ub-HA), with whole-cell extracts collected for immunoprecipitation with anti-GFP antibody, followed by immunoblot analysis with anti-HA antibody that detected ubiquitin. FIG. 6B is an image of a Western blot of whole-cell extracts of HEK 293T cells transfected with tauWT-GFP and treated with 2 mM and 10 mM lactate for 24 hours, with whole-cell extracts collected for cleavage assay with rAEP, followed by immunoblot analysis that detected tau by using tau5 antibody. FIG. 6C is an image of a Western blot of whole-cell extracts of HEK 293T cells transfected with tauWT-GFP, control and shRNA against LDHA, and Ub-HA, with whole-cell extracts collected for immunoprecipitation with anti-GFP antibody, followed by immunoblot analysis with anti-HA antibody that detected ubiquitin. FIG. 6D is an image of a Western blot of whole-cell extracts of HEK 293T cells transfected with tauWT-GFP with control and shRNA against LDHA, with whole-cell extracts collected for cleavage assay with rAEP treatment, followed by immunoblot analysis that detected tau by using tau5 antibody. FIG. 6E is an image of a Western blot of whole-cell extracts of HEK 293T cells transfected with tauWT-383, tau3KR and Ub-HA, with whole-cell extracts collected for immunoprecipitation with anti-tau5 antibody, followed by immunoblot analysis with anti-HA antibody that detected ubiquitin. FIG. 6F is an image of a Western blot of whole-cell extracts of HEK 293T cells transfected with tauWT-383 and tau3KR, with whole-cell extracts collected for tau cleavage assay with rAEP treatment, followed by immunoblot analysis that detected tau by using tau5 antibody. FIG. 6G is an image of a Western blot of an in vitro ubiquitination assay, where purified non-lactyl-tau and lactyl-tau proteins were incubated with ubiquitination proteins (E1, E2, CHIP, and ubiquitin), followed by immunoblotting with anti-ubiquitin (Ub) and tau5 antibodies. FIG. 6H is an image of a Western blot of an in vitro cleavage assay, where purified non-lactyl-tau and lactyl-tau proteins were incubated with rAEP, followed by immunoblotting with tau5 and tau368N antibodies.

FIGS. 7A-7B illustrate binding of lactylated tau (K281) polyclonal antibodies. FIG. 7A is an image of a Western blot of whole-cell extracts of wild-type and PS19 mouse hippocampal lysate using indicated antibodies against TauK281la; AT8; Tau368N; Tau5; and Actin (Control), respectively. FIG. 7B is an image of a Western blot of whole-cell extracts of AD and non-AD human brain cell lysate using indicated antibodies against TauK281la; Tau368N; Tau5; and Actin (Control), respectively.

FIG. 8 is a violin plot showing the effects of lactylated peptide A and lactylated peptide B including lactylated K281 (K281la), and non-lactylated control (Ctrl) peptides on the presence of pathogenic tau in the brains of PS19 and Normal (Ctrl) test animals, respectively.

### DETAILED DESCRIPTION OF THE INVENTION

### I. Definitions

As used herein, “tauopathy” refers to a neurodegenerative disease associated with the pathological aggregation of tau protein in the human brain.

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multispecific antibodies (e.g., bispecific antibodies), and antibody fragments so long as they specifically bind the target antigen. Any specific antibody can be used in the methods and compositions provided herein. Thus, in one form the term “antibody” encompasses a molecule including at least one variable region from a light chain immunoglobulin molecule and at least one variable region from a heavy chain molecule that in combination form a specific binding site for the target antigen. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia, C. et al. (1987) “Canonical Structures For The Hypervariable Regions Of Immunoglobulins,” J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to the described antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass. An “antibody fragment” or “antigen binding fragment” of an antibody is defined as at least a portion of the variable region of the immunoglobulin molecule that binds to its target, i.e., the antigen binding region (also antigen binding domain). In one form it specifically covers single antibodies and clones thereof and anti-antibody compositions with poly-epitopic specificity. The antibody of the present methods and compositions can be monoclonal or polyclonal. An antibody can be in the form of an antigen binding antibody fragment including a Fab fragment, F(ab′)₂ fragment, a single chain variable region, and the like. Fragments of intact molecules can be generated using methods well known in the art and include enzymatic digestion and recombinant means. Thus, the “fragment” may be a recombinant protein, e.g., a fusion protein.

As used herein, any form of the “antigen” can be used to generate an antibody that is specific for the target antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one form, the antibody of the methods and compositions herein specifically bind at least a portion of the extracellular domain of the target antigen of interest.

The antibodies or antigen binding fragments thereof provided herein may be conjugated to a “bioactive agent.” As used herein, the term “bioactive agent” refers to any synthetic or naturally occurring compound that binds the antigen and/or enhances or mediates a desired biological effect.

In one form, the binding fragments are biologically active fragments. As used herein, the term “biologically active” refers to an antibody or antibody fragment that is capable of binding the desired the antigenic epitope and directly or indirectly exerting a biologic effect.

“Bispecific” antibodies are also useful in the present methods and compositions. As used herein, the term “bispecific antibody” refers to an antibody, typically a monoclonal antibody, having binding specificities for at least two different antigenic epitopes. In one form, the epitopes are from the same antigen. In another form, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison et al., Proc. Natl. Acad. Sci. USA 81: 6851-6855 (1984)).

The term “specifically binds” or “immuno-specifically binds” refers to the binding of an antibody to its cognate antigen while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The term “monoclonal antibody” or “mAb” refers to an antibody obtained from a substantially homogeneous population of antibodies, i.e., the individual antibodies within the population are identical except for possible naturally occurring mutations that may be present in a small subset of the antibody molecules.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

“Treatment” or “treating” means to administer a composition to a subject or a system with an undesired condition (e.g., AD). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., AD). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the terms “variant” or “active variant” refers to a polypeptide or polynucleotide that differs from a reference polypeptide or polynucleotide, but retains necessary properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

Compositions for detection of lactylated tau protein have been developed. As described in the Examples, a single nuclei RNA-seq (snRNA-seq) dataset was analyzed from control (ND) and AD human brain samples to identify AD-associated changes in the expression of lactate signature genes (genes encoding regulators of lactate metabolism). It has been established that cells in the brain of AD patients, including inhibitory (INH) neurons, excitatory (EX) neurons, oligodendrocytes (ODC), oligodendrocyte progenitor cell (OPC), and microglia (MG) clusters exhibit a significant and coordinated upregulation of lactate signature gene expression. Further, it has been established that lactate signature expression is up-regulated in AD CSF, and a significant increase in the number of cells positive for an antibody against lactylated lysine (Kla) was observed. The data support the conclusion that the presence and quantity of lactylated tau proteins can serve as biomarkers for the detection of tauopathies in human. Therefore, lactylated lysine associated with tau protein has been established as a marked for AD.

Accordingly, compositions for the detection of lactylated tau protein are provided. In some forms, the compositions are binding proteins, such as antibodies or antigen-binding fragments or fusion-proteins thereof. In other forms, the compositions are non-protein molecules, such as nucleic acids or small molecules.

Any of the described compositions for the detection of lactylated tau protein motifs can further include a carrier protein fused or conjugated thereof, optionally to the N- or C-terminus of the peptide. As discussed elsewhere in more detail, also provided are nucleic acid molecules (DNA or RNA) that encode any of the described compositions for the detection of lactylated tau protein, as well as fusion proteins or fragments, as well as vector molecules (such as plasmids) that are capable of transmitting or of replication of such nucleic acid molecules. The nucleic acids can be single-stranded, double-stranded, may contain both single-stranded and double-stranded portions.

Pharmaceutical compositions including an effective amount of the described compositions for the detection of lactylated tau protein and optionally further including one or more additional agents are also provided.

### A. Microtubule Associated Protein (Tau)

Microtubule associated protein tau (MAPT) (also referred to herein as tau) is located on chromosome 17q21 in humans, containing 16 exons. The major tau protein in the human brain is encoded by 11 exons, of which exons 2, 3 and 10 are alternatively spliced, leading to the formation of six tau isoforms.

In the human brain, tau proteins constitute a family of six isoforms with a range of 352-441 amino acids, which differ in having either zero, one, or two inserts of 29 amino acids at the N-terminal part (exons 2 and 3) and three or four repeat-regions at the C-terminal part (exon 10). Thus, the longest isoform in the CNS has four repeats (R1, R2, R3 and R4) and two inserts (441 amino acids total), while the shortest isoform has three repeats (R1, R3 and R4) and no insert (352 amino acids total).

Tau is profoundly regulated by post-translational modification (PTM), for example, phosphorylation, ubiquitination, and acetylation, and is profoundly implicated in tauopathy. For example, pathologies and dementias of the nervous system such as Alzheimer's disease and Parkinson's disease are associated with tau proteins that have become hyperphosphorylated insoluble aggregates, which accumulate inside cells as neurofibrillary tangles (NFTs). Tangle pathology is most pronounced in vulnerable brain regions in AD, including the cortex and hippocampus.

### 1. Tau Reference Polypeptide Sequences

Full-length proteins sequences for the human tau are known in the art. See e.g., UniPro Accession No. P10636·TAU_HUMAN, which is specifically incorporated by reference herein in its entirety, and provides the amino acid sequence for normal human tau protein:

(SEQ ID NO: 1, with sites of potentially
lactylated lysine residues is
indicated in bold font)
MAEPRQEFEVMEDHAGTYGLGDRKDQGGYTMHQDQEGDTDAG
LKESPLQTPTEDGSEEPGSETSDAKSTPTAEDVTAPLVDEGA
PGKQAAAQPHTEIPEGTTAEEAGIGDTPSLEDEAAGHVTQEP
ESGKVVQEGFLREPGPPGLSHQLMSGMPGAPLLPEGPREATR
QPSGTGPEDTEGGRHAPELLKHQLLGDLHQEGPPLKGAGGKE
RPGSKEEVDEDRDVDESSPQDSPPSKASPAQDGRPPQTAARE
ATSIPGFPAEGAIPLPVDFLSKVSTEIPASEPDGPSVGRAKG
QDAPLEFTFHVEITPNVQKEQAHSEEHLGRAAFPGAPGEGPE
ARGPSLGEDTKEADLPEPSEKQPAAAPRGKPVSRVPQLKARM
VSKSKDGTGSDDKKAKTSTRSSAKTLKNRPCLSPKHPTPGSS
DPLIQPSSPAVCPEPPSSPKYVSSVTSRTGSSGAKEMKLKGA
DGKTKIATPRGAAPPGQKGQANATRIPAKTPPAPKTPPSSGE
PPKSGDRSGYSSPGSPGTPGSRSRTPSLPTPPTREPKKVAVV
RTPPKSPSSAKSRLQTAPVPMPDLKNVKSKIGSTENLKHQPG
GGKVQIINKKLDLSNVQSKCGSKDNIKHVPGGGSVQIVYKPV
DLSKVTSKCGSLGNIHHKPGGGQVEVKSEKLDFKDRVQSKIG
SLDNITHVPGGGNKKIETHKLTFRENAKAKTDHGAEIVYKSP
VVSGDTSPRHLSNVSSTGSIDMVDSPQLATLADEVSASLAKQ
GL.

In some forms, the tau protein includes a tau 441 peptide having the amino acid sequence:

(SEQ ID NO: 2; Tau 441, with sites of
potentially lactylated lysine residues
is indicated in bold font)
MAEPRQEFEVMEDHAGTYGLGDRKDQGGYTMHQDQEGDTDAG
LKESPLQTPTEDGSEEPGSETSDAKSTPTAEDVTAPLVDEGA
PGKQAAAQPHTEIPEGTTAEEAGIGDTPSLEDEAAGHVTQAR
MVSKSKDGTGSDDKKAKGADGKTKIATPRGAAPPGQKGQANA
TRIPAKTPPAPKTPPSSGEPPKSGDRSGYSSPGSPGTPGSRS
RTPSLPTPPTREPKKVAVVRTPPKSPSSAKSRLQTAPVPMPD
LKNVKSKIGSTENLKHQPGGGKVQIINKKLDLSNVQSKCGSK
DNIKHVPGGGSVQIVYKPVDLSKVTSKCGSLGNIHHKPGGGQ
VEVKSEKLDFKDRVQSKIGSLDNITHVPGGGNKKIETHKLTF
RENAKAKTDHGAEIVYKSPVVSGDTSPRHLSNVSSTGSIDMV
DSPQLATLADEVSASLAKQGL.

Compositions including engineered non-lactylated tau proteins and polypeptides are also described. The engineered nonlactylated tau proteins typically include a sequence of about 20 to about 441, or about 30 to about 400, or 30 to about 331, or about 40 to about 200, or about 40 to about 100 contiguous amino acid residues, inclusive, of any one of SEQ ID NOS:1, or 2. For example, in some forms, the engineered lactylated tau protein includes a sequence of 30, 31, 32, 33, 34, 35, 36, 37, 38, 39, 40, 41, 42, 43, 44, 45, 46, 46, 47, 48, 49, 50, 51, 52, 53, 54, 55, 56, 57, 58, 59, 60, 61, 62, 63, 64, 65, 66, 67, 68, 69, or 70, contiguous amino acid residues, that include at least one non-lactylated lysine residue, for example at one or more positions including K224, K225, K257, K267, K281, K311, K317, K321 and K331 of SEQ ID NO:2. In exemplary forms, the engineered non-lactylated tau protein is effective to cap and arrest formation of neurofibrillary tangles (NFTs), and/or to specifically target pathological lactylated tau aggregates without affecting physiological tau functions. In some forms, the engineered non-lactylated tau protein includes a non-lactylated lysine residue at position 331 of SEQ ID NO:2.

### 2. Lactylated Tau Proteins

It has been established that lactylation is a PTM of tau implicated in the development and pathology of tauopathies, such as AD.

Lysine lactylation is a post-translational modification (PTM) that influences a variety of cellular processes, including cell metabolism, neuronal development, cellular reprogramming, inflammation, and tumorigenesis. This PTM has three distinct isomers: L-lactylation (KL-la), D-lactylation (KD-la), and N-ε-(carboxyethyl)-Lysine (Kce). The cellular levels of KL-la and KD-la can be stimulated by the two optical isomers of lactate, i.e., L-lactate and D-lactate, respectively. As a byproduct of cellular metabolism, lactate serves as an important signaling molecule, regulating tumor development and immune responses. Lysine lactylation has mechanistic similarities with lysine acylation. To introduce and remove the modification, there are generally two distinct types of catalytic mechanisms, enzymatic and non-enzymatic. The enzymatic mechanism is conducted by lactyltransferases (writers) and delactylases (erasers), which function similarly to acetyltransferases (KATs) and deacetylases. KL-la is tightly regulated by these enzymes, which can install and remove the L-lactyl groups rather than the acetyl group to lysine residues. Studies in Escherichia coli (E. coli) and Streptococcus mutans (S. mutans) have shown that, lysine lactylation is a PTM conserved across prokaryotes and eukaryotes, much like lysine acetylation. Furthermore, several lactyltransferases and delactylases have been identified in various microbial species. On the other hand, a nonenzymatic mechanism has been identified in HEK293 cells, where S-D-lactylglutathione (LGSH) directly donates its D-lactyl group to lysine residues, generating KD-la modification.

Elevated tau lactylation in human AD brain samples include lysine residue at position 331 (K331) being a prominent site that is associated with AD pathogenesis. The data in the Examples suggest that lactylation regulates tau phosphorylation, ubiquitination, turnover, and cleavage, such that that increased lactate levels in AD brains may drive tau lactylation, potentially contributing to tauopathy. Quantitative proteomics analysis of tau lactylation in human control and AD brain samples identified six lysine sites to be lactylated, including K257, K267, K281, K311, K317, K331, and possibly K321 of human Tau 441 (SEQ ID NO:2).

Therefore, compositions of lactylated tau proteins and fragments or variants thereof are provided. In some forms, the compositions include isolated or recombinant tau proteins including one or more lactylayted lysine residues. For example, in some forms, the lactylated tau proteins include one or more lactylayted lysine residue, for example, such as one or more lactylate residue selected from K257, K267, K281, K311, K317, K331, and K321. In some forms, the lactylated tau protein includes a lactylayted lysine residue at K257 of SEQ ID NO:2. In some forms, the lactylated tau protein includes a lactylayted lysine residue at K267 of SEQ ID NO:2. In some forms, the lactylated tau protein includes a lactylayted lysine residue at K281 of SEQ ID NO:2. In some forms, the lactylated tau protein includes a lactylayted lysine residue at K311 of SEQ ID NO:2. In some forms, the lactylated tau protein includes a lactylayted lysine residue at K317 of SEQ ID NO:2. In some forms, the lactylated tau protein includes a lactylayted lysine residue at K331 of SEQ ID NO:2. In some forms, the lactylated tau protein includes a lactylayted lysine residue at K321 of SEQ ID NO:2.

In exemplary forms, the lactylated tau protein includes a variant of the amino acid sequence of SEQ ID NO:2, including lactylation of all of the lysine residues at positions K224, K225, K257, K267, K281, K311, K317, K331, and K321 of SEQ ID NO:2:

(SEQ ID NO: 3; lactylated lysine residues
are indicated as “(K-lactyl)”),
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREP (K-lactyl) (K-lactyl)
VAVVRT PPKSPSSAKSRLQTAPVPMPDLKNV (K-lactyl)
SKIGSTENL (K-lactyl) HQPGGG KVQIINK (K-
lactyl) LDLSNVQSKCGSKDNIKHVPGGGSVQIVY (K-lactyl)
PVDLS (K-lactyl) VTS (K-lactyl)
CGSLGNIHH (K-lactyl) PGGGQVEVKSEKLD
FKDRVQSKIGSLDNITHVPGGGNKKIETHKLTFRENAKAKTDHGAEIV
YKSPVVSGDTSPRHLSNVSSTGSIDMVDSPQLATLADEVSASLAKQGL.
(SEQ ID NO: 33)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGPPKSGDRSGYSSPE
GSPGTPGSRSRTPSLPTPPTREPK (K-lactyl)
VAVVRTPPKSPSSAKS
RLQTAPVPMPDLKNVKSKIGSTENLKHQPGGGKVQIINKKLDLSNVQSKC
GSKDNIKHVPGGGSVQIVYKPVDLSKVTSKCGSLGNIHHKPGGGQVEVKS
EKLDFKDRVQSKIGSLDNITHVPGGGNKKIETHKLTFRENAKAKTDHGAE
IVYKSPVVSGDTSPRHLSNVSSTGSIDMVDSPQLATLADEVSASLAKQG
L.

or a variant or fragment thereof.

In exemplary forms, the lactylated tau protein includes a variant of the amino acid sequence of SEQ ID NO:2, including lactylation of the lysine residue at position K224, of SEQ ID NO:2:

(SEQ ID NO: 32)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREP(K-lactyl)KVAVVRTPPKSPSSAKS
RLQTAPVPMPDLKNVKSKIGSTENLKHQPGGGKVQIINKKLDLSNVQSKC
GSKDNIKHVPGGGSVQIVYKPVDLSKVTSKCGSLGNIHHKPGGGQVEVKS
EKLDFKDRVQSKIGSLDNITHVPGGGNKKIETHKLTFRENAKAKTDHGAE
IVYKSPVVSGDTSPRHLSNVSSTGSIDMVDSPQLATLADEVSASLAKQG
L.

In exemplary forms, the lactylated tau protein includes a variant of the amino acid sequence of SEQ ID NO:2, including lactylation of the lysine residue at position K225 of SEQ ID NO:2:

(SEQ ID NO: 33)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPK(K-lactyl)VAVVRTPPKSPSSAKS
RLQTAPVPMPDLKNVKSKIGSTENLKHQPGGGKVQIINKKLDLSNVQSKC
GSKDNIKHVPGGGSVQIVYKPVDLSKVTSKCGSLGNIHHKPGGGQVEVKS
EKLDFKDRVQSKIGSLDNITHVPGGGNKKIETHKLTFRENAKAKTDHGAE
IVYKSPVVSGDTSPRHLSNVSSTGSIDMVDSPQLATLADEVSASLAKQGL.

In exemplary forms, the lactylated tau protein includes a variant of the amino acid sequence of SEQ ID NO:2, including lactylation of the lysine residue at position K257 of SEQ ID NO:2:

(SEQ ID NO: 34)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNV (K-lactyl) SKIGSTENLKHQPGGGKVQIINKKLDLSNVQS
KCGSKDNIKHVPGGGSVQIVYKPVDLSKVTSKCGSLGNIHHKPGGGQVEV
KSEKLDEKDRVQSKIGSLDNITHVPGGGNKKIETHKLTFRENAKAKTDHG
AEIVYKSPVVSGDTSPRHLSNVSSTGSIDMVDSPQLATLADEVSASLAKQ
GL.

In exemplary forms, the lactylated tau protein includes a variant of the amino acid sequence of SEQ ID NO:2, including lactylation of all of the lysine residue at position K267, of SEQ ID NO: 2:

(SEQ ID NO: 35)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENL (K-lactyl) HQPGGGKVQIINKKLDLSNVQS
KCGSKDNIKHVPGGGSVQIVYKPVDLSKVTSKCGSLGNIHHKPGGGQVEV
KSEKLDFKDRVQSKIGSLDNITHVPGGGNKKIETHKLTFRENAKAKTDHG
AEIVYKSPVVSGDTSPRHLSNVSSTGSIDMVDSPQLATLADEVSASLAKQ
GL.

In exemplary forms, the lactylated tau protein includes a variant of the amino acid sequence of SEQ ID NO:2, including lactylation of all of the lysine residue at position K311 of SEQ ID NO: 2:

(SEQ ID NO: 36)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVY
(K-lactyl)
PVDLSKVTSKCGSLGNIHHKPGGGQVEVKSEKLDFKDRVQSKIGSLDNIT
HVPGGG
NKKIETHKLTFRENAKAKTDHGAEIVYKSPVVSGDTSPRHLSNVSSTGSI
DMVDSPQLATLADEVSASLAKQGL.

In exemplary forms, the lactylated tau protein includes a variant of the amino acid sequence of SEQ ID NO:2, including lactylation of all of the lysine residue at position K317, of SEQ ID NO: 2:

(SEQ ID NO: 37)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVY
KPVDLS (K-lactyl)
VTSKCGSLGNIHHKPGGGQVEVKSEKLDFKDRVQSKIGSLDNITHVPGGG
NKKIETHKLTFRENAKAKTDHGAEIVYKSPVVSGDTSPRHLSNVSSTGSI
DMVDSPQLATLADEVSASLAKQGL.

In exemplary forms, the lactylated tau protein includes a variant of the amino acid sequence of SEQ ID NO:2, including lactylation of all of the lysine residue at position K331 of SEQ ID NO: 2:

(SEQ ID NO: 38)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTS
(K-lactyl)
CGSLGNIHHKPGGGQVEVKSEKLDFKDRVQSKIGSLDNIT
HVPGGGNKKIETHKLTFRENAKAKTDHGAEIVYKSPVVSGDTSPRHLSNV
SSTGSIDMVDSPQLATLADEVSASLAKQGL.

In exemplary forms, the lactylated tau protein includes a variant of the amino acid sequence of SEQ ID NO:2, including lactylation of the lysine residue at position K321 of SEQ ID NO:2:

(SEQ ID NO: 39)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHH (K-
lactyl) PGGGQVEVKSEKLDFKDRVQSKIGSLDNITHVPGGGN
KKIETHKLTFRENAKAKTDHGAEIVYKSPVVSGDTSPRHLSNVSSTGSID
MVDSPQLATLADEVSASLAKQGL.

In exemplary forms, the lactylated tau protein includes a variant of the amino acid sequence of SEQ ID NO:2, including lactylation of the lysine residue at position K281 of SEQ ID NO:2:

(SEQ ID NO: 40)
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINK (K-
lactyl) LDLSNVQSKCGSKDNIKHVPGGGSVQIVYKPVDLSKVTSKCG
SLGNIHHKPGGGQVEVKSEKLDFKDRVQSKIGSLDNITHVPGGGNKKIET
HKLTFRENAKAKTDHGAEIVYKSPVVSGDTSPRHLSNVSSTGSIDMVDSP
QLATLADEVSASLAKQGL.

Compositions including engineered lactylated tau proteins are also described. The engineered lactylated tau protein typically include a sequence of about 20 to about 441, or about 30 to about 400, or 30 to about 331, or about 40 to about 281, or about 40 to about 267, or about 40 to about 257, or about 40 to about 225, or about 40 to about 224 contiguous amino acid residues, inclusive, of any one of SEQ ID NOS:3, or 32-40. For example, in some forms, the engineered lactylated tau protein includes a sequence of 30, 31, 32, 33, 34, 35, 36, 37, 38, 39, 40, 41, 42, 43, 44, 45, 46, 46, 47, 48, 49, 50, 51, 52, 53, 54, 55, 56, 57, 58, 59, 60, 61, 62, 63, 64, 65, 66, 67, 68, 69, or 70, contiguous amino acid residues, that include at least one lactylated lysine residue, for example at one or more positions including K224, K225, K257, K267, K281, K311, K317, K321, and K331 of SEQ ID NO:2.

3. Peptide Fragments of Tau Proteins with Defined Lactylation Sites

Full length and fragments of lactylated tau proteins can be used to raise polyclonal and monoclonal antibodies specific for lactylation at one or more residues of tau. Furthermore, as set forth in the Examples, it has been established that peptide fragments of lactylated tau proteins that include defined lactylation sites can reduce the amount, formation or distribution of tau fibril. Therefore, peptide fragments of lactylated tau proteins that include defined lactylation sites are provided as therapeutic agents for the treatment and prevention of tauopathies.

In some forms, the peptide fragments of lactylated tau proteins that include defined lactylation sites, include lactyllysine at position 225 of Tau 441 (K2251a), such as:

(SEQ ID NO: 4; Tau K224/K225la peptide A 216-230)
CPTPPTREP (K-lactyl) (K-lactyl) VAVVR;
and
(SEQ ID NO: 5; Tau K224/K225la peptide B 218-230)
PPTREP (K-lactyl) (K-lactyl) VAVVRC.

In some forms, the peptide fragments of lactylated tau proteins that include defined lactylation sites, include lactyllysine at position 281 of Tau 441 (K281la), such as:

(SEQ ID NO: 7; Tau K281la peptide A: 276-286)
CQIINK (K-lactyl) LDLSN;
and
(SEQ ID NO: 8; Tau K281la peptide B: 279-290)
CINK (K-lactyl) LDLSNVQSKC.

In some forms, the peptide fragments of lactylated tau proteins that include defined lactylation sites, include lactyllysine at position 331 of Tau 441 (K331la), such as:

(SEQ ID NO: 10; Tau K33 1la peptide A: 326-338)
GNIHH (K-lactyl) PGGGQVEC;
and
(SEQ ID NO: 11; Tau K331la peptide B: 326-336)
GNIHH (K-lactyl) PGGGQC.

In some forms, the peptide fragments of lactylated tau proteins that include defined lactylation sites, include lactyllysine at position 370 of Tau 441 (K3701a), such as:

(SEQ ID NO: 13; Tau K3691a/K370la peptide A:
361-375)
CTHVPGGGN (K-lactyl) (K-lactyl) IETHK;
and
(SEQ ID NO: 14; Tau K369la/K370la peptide B:
363-375).
CVPGGGN (K-lactyl) (K-lactyl) IETHK.

In some forms, the peptides include a lactylated tau polypeptide that includes one or more lactyllysines within the sequence of any one of SEQ ID NOs. 3, 4-5, 7-8, 10-11, 13-14, or 32-40, such as a fragment thereof having from about 2 to about 440 amino acids, inclusive. For example, in some forms, the lactylated tau polypeptide includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 or more than 100 consecutive amino acids of any one of SEQ ID NOs. 3, 4-5, 7-8, 10-11, 13-14, or 32-40, and which include at least one lactylated lysine residue.

### 4. Sources of Tau Proteins

Samples including tau proteins for analysis according to the described methods are also provided. In some forms, the source of a tau protein for analysis is a biological sample obtained from a subject. Methods for preparation of biological samples, such as those including cells and/or tissues that may include tau proteins are known in the art. For example, methods for collecting various bodily or cellular samples and for extracting one or more components are well known in the art. In some forms, biological samples include cells, tissues, and/or bodily fluids containing tau proteins. Exemplary source organisms for biological samples include animals, such as humans.

Exemplary biological samples include bodily materials including, but not limited to, cerebro-spinal fluid (CSF), tissue, organ(s), blood, lymph, urine, gynecological fluids, and biopsies. Bodily fluids can include CSF, serum, blood, urine, saliva, or any other bodily secretion or derivative thereof. Blood can include whole blood, plasma, serum, or any derivative of blood. Typically, the sample includes cells, particularly eukaryotic cells from tissue(s) or from a biopsy. Samples can be obtained from a subject by a variety of techniques including, for example, by scraping, washing, or swabbing an area, by using a needle to aspirate bodily fluids, or by removing a tissue sample (i.e., biopsy).

If the tau protein is within cells, tissue or bodily fluids, preparation and purification of the tau proteins from the sample can include lysis of cells, such as cells within blood. A biological sample, such as a CSF sample obtained from a subject can be obtained, processed, (e.g., sterilized) and provided for the described methods as a fresh or frozen aqueous solution, or as a lyophilized, dry powder using techniques and procedures known in the art. In some forms, the methods include obtaining more than one sample including tau protein from a subject, for example, two or more samples, for example, separated in time. In some forms, a sample includes a known amount of lactlylated tau, or non-lactylated tau, for example, for use as a control in the described methods. In some forms, a sample includes brain tissue, bone marrow, CSF, blood, saliva or urine. In some forms, a sample includes a known amount of lactylated tau from diseased cells, such as cells from an AD brain.

In other forms, a sample includes tau protein, such as a lactylated tau protein, from an artificial source, such as a recombinant tau protein or a fragment thereof, for example, a recombinant lactylated tau protein.

### B. Binding Compositions Specific for Lactylated Tau Protein Motifs

Binding compositions, such as binding proteins that specifically and selectively recognize and bind to lactylated tau protein motifs are provided. Typically, the specific binding compositions do not recognize or bind to non-lactylated tau protein, or do not bind to non-lactylated tau protein with equivalent affinity and/or avidity. Exemplary binding compositions include binding proteins, such as binding proteins that bind to one or more structures on lactylated tau protein, and which are not present on non-lactylated tau protein. Exemplary binding molecules include proteins, such as antibodies, such as polyclonal antibodies or monoclonal antibodies. In some forms, the binding proteins include antibodies which selectively bind to lactylated lysine residues per se. In other forms, the binding proteins include antibodies which selectively bind to lactylated lysine residues that are present on lactylated tau protein, but do not bind to lactylated lysine residues that are not associated with tau protein.

### 1. Antibodies Specific for Lactylated Lysine (Kla)

Antibodies specific for lactylated lysine (Kla) targets are provided.

Existing polyclonal antibodies to lactylated lysine (Kla) targets were raised against non-tau-based epitopes that in some forms may be limited in specificity and may give rise to non-tau-specific detection, for example, of proteins harboring similar Kla motifs. In some forms, the anti-lactyllysine antibodies, or antigen binding fragments or binding proteins derived therefrom detect proteins with L-lactylated lysine residues, regardless of the surrounding peptide sequences. In some forms, anti-lactyllysine antibodies, or antigen binding fragments or binding proteins derived therefrom do not bind with D-lactylated or N-ε-carboxyethylated peptides.

Antibodies specific for lactylated lysine (Kla) targets are available from multiple commercial sources, including from PTM BIO; see, e.g., monoclonal rabbit IgG anti-L-Lactyllysine antibody derived from clone number: 9H1L6 (Cat. #PTM-1401RM; see Table 1), which detects proteins with L-lactylated lysine residues, regardless of the surrounding peptide sequences, and does not cross-react with D-lactylated or N-ε-carboxyethylated peptides.

Another commercially available monoclonal anti-L-Lactyllysine antibody is the mouse IgG3 mAb derived from clone number: K16286_4E11 (see antibody registry accession No. AB_3668225; available from Invitrogen; see cat #. MA5-55993).

Commercially available polyclonal anti-Lactic acid Lysine antibodies include rabbit, antibody registry accession No. AB_2901531 (Invitrogen cat #. PA5-116901), as well as mouse IgG anti-lactyllysine, antibody registry accession No. AB_3667847 (Invitrogen cat #. MA5-55615).

In addition to those antibodies listed, the described methods may use any anti-lactyllysine (Kla) antibody that specifically recognizes lactylated lysine residues, including but not limited to pan-anti-lactyllysine antibodies and site-specific anti-lactyllysine antibodies (e.g., antibodies recognizing lactylated tau at specific lysine residues). Such antibodies may be polyclonal or monoclonal, and may be generated in any host species or produced recombinantly. Commercially available anti-lactyllysine antibodies (e.g., from vendors including PTM Bio, Proteintech, Abcam, and Cell Signaling Technology) as well as custom-generated antibodies are contemplated and suitable for use in the disclosed methods.

### 2. Antibodies Specific for Lactylated Tau Protein Motifs

Antibodies specific for lactylated tau protein motifs are provided. It may be that existing reagents for detecting lactylated lysine (Kla) targets are insufficient to use for detection and diagnostic purposes with high confidence.

Therefore, in some forms, improved antibodies that specifically and selectively bind to lactylated tau protein motifs are provided to detect lactylated tau protein biomarkers for use in diagnostic and prognostic applications. Therefore, disclosed are antibodies and their antigen binding fragments and other molecules that are capable of immuno-specifically binding lactylated tau protein proteins present within a biological sample obtained from a subject. More particularly, antibodies and their antigen binding fragments and other molecules capable of immunospecifically binding one or more lactylated tau proteins are provided.

i. Sources Antibodies Specific for Lactylated Tau

Polyclonal and monoclonal antibodies (mAbs) that selectively and specifically bind to lactylated tau proteins, and which are produced according to the following methods of making antibodies are also provided.

In an exemplary form, a polyclonal antibody is derived by immunization of a host animal with a synthetic peptide comprising a lactylated lysine residue corresponding to a tau peptide sequence, conjugated to a carrier protein to enhance immunogenicity. The peptide included the lactylated lysine modification at the target position, and the resulting antiserum is then purified to enrich for antibodies recognizing lactylated tau.

In some forms, the first step for production of polyclonal and monoclonal antibodies (mAbs) that selectively and specifically bind to lactylated tau proteins is immunization of an appropriate host with a suitable antigen, such as a lactylated tau protein, or a fragment thereof. A given composition for immunization may vary in its immunogenicity. It is often necessary therefore to boost the host immune system, as may be achieved by coupling a peptide or polypeptide antigen to a carrier. Exemplary and preferred carriers are discussed below. Means for conjugating a polypeptide to a carrier protein are well known in the art and include glutaraldehyde, m-maleimidobencoyl-N-hydroxysuccinimide ester, carbodiimide and bis-biazotized benzidine.

The immunogenicity of a particular antigen composition can be enhanced by the use of non-specific stimulators of the immune response, known as adjuvants. The amount of antigen composition used in the production of polyclonal antibodies varies upon the nature of the antigen as well as the animal used for immunization. A variety of routes can be used to administer the antigen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate monoclonal antibodies. Following immunization, somatic cells with the potential for producing antibodies, specifically B lymphocytes (B cells), are selected for use in the mAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Methods for generating hybrids of antibody-producing spleen or lymph node cells and myeloma cells usually include mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

A preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radio-immunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

Monoclonal antibodies produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Yeast-based antibody presentation libraries may be designed rationally, and antibodies may be selected and/or isolated from such yeast-based antibody presentation libraries, as disclosed in, for example, WO2012/009568; WO2009/036379; WO2010/105256; WO2003/074679; U.S. Pat. Nos. 8,691,730; and 9,354,228 The antibodies may then be expressed as full length IgGs from the desired cell type and purified.

In some forms, the methods include one or more steps for selecting an screening an antibody produced according to the described methods. For example, in some forms, the methods select an antibody for specificity and/or selectivity to bind a lactylated tau protein, or a fragment thereof that includes at least one lactylated lysine. In some forms, the methods select an antibody that does not bind to a reference or control protein, such as a tau protein that does not include a lactylated lysine.

a. Peptide Antigens

Provided are peptide antigens that can be used to raise antibodies against lactylated tau proteins. Also provided are non-lactylated tau peptides that can be used as antigens for control antibodies. Typically, the peptide antigens include a lactylated tau antigenic peptide having a sequence of amino acids that includes at least one lactylated lysine. In some forms, lactylated tau antigen is between about 10 and about 20 amino acids, or between about 15 and 18 amino acids, or about 16 amino acids in length. Typically the lactylated tau antigenic peptide includes one, two, or three lactylated residues, such as one, two or three lactylated lysine residues. In particular, the lactylated residue(s) are necessary to insure reactivity of the resulting antibody with the target lactylated tau protein, and selectivity against the non-lactylated (reference) amino acid sequence.

In some forms, the antibodies are raised against a lactylated tau protein, for example, including lactyalation of one or more or the lysine residues of the proteins having an amino acid sequence set forth in any of SEQ ID NOs:1 or 2. For example, in some forms, the antibodies are raised against a lactylated tau protein having an amino acid sequence of SEQ ID NO:3, or 32-40. In an exemplary form, antibodies against a lactyalted tau protein are raised against a lactylated tau protein including a lactyllysine at position K331 of SEQ ID NO:2, e.g., as set forth in SEQ ID NO:36.

In some forms, the antibodies immunospecifically bind to one or more polypeptide motifs present on lactylated tau protein, including:

(SEQ ID NO: 4; Tau K224/K225la peptide A
216-230)
CPTPPTREP (K-lactyl) (K-lactyl) VAVVR;
and
(SEQ ID NO: 5; Tau K224/K225la peptide B
218-230)
PPTREP (K-lactyl) (K-lactyl) VAVVRC.

In some forms, antibodies that immunospecifically bind to SEQ ID NO:4 and/or 5 do not bind to a non-lactylated protein motif including:

(SEQ ID NO: 6; unmodified Tau K224/K225la
peptide B 218-230)
CPTPPTREPKKVAVVR.

In some forms, the antibodies immunospecifically bind to one or more polypeptide motifs present on lactylated tau protein, including:

(SEQ ID NO: 7; Tau K281la peptide A: 276-286)
CQIINK (K-lactyl) LDLSN;
and
(SEQ ID NO: 8; Tau K281la peptide B: 279-290)
CINK (K-lactyl) LDLSNVQSKC.

In some forms, antibodies that immunospecifically bind to SEQ ID NO:7 and/or 8 do not bind to a non-lactylated protein motif including:

(SEQ ID NO: 9; Tau K281la unmodified peptide:
279-290)
CQIINKKLDLSN.

In some forms, the antibodies immunospecifically bind to one or more polypeptide motifs present on lactylated tau protein, including:

(SEQ ID NO: 10; Tau K331la peptide A: 326-338)
GNIHH (K-lactyl) PGGGQVEC;
and
(SEQ ID NO: 11; Tau K331la peptide B: 326-336)
GNIHH (K-lactyl) PGGGQC.

In some forms, antibodies that immunospecifically bind to SEQ ID NO: 10 and/or 11 do not bind to a non-lactylated protein motif including:

(SEQ ID NO: 12; Unmodified Tau K331la peptide:
326-338)
GNIHHKPGGGQVEC.

In some forms, the antibodies immunospecifically bind to one or more polypeptide motifs present on lactylated tau protein, including:

(SEQ ID NO: 13; Tau K369/K370la
peptide A: 361-375)
CTHVPGGGN (K-lactyl) (K-lactyl) IETHK;
and
(SEQ ID NO: 14; Tau K331la peptide B:
363-375)
CVPGGGN (K-lactyl) (K-lactyl) IETHK.

In some forms, antibodies that immunospecifically bind to SEQ ID NO: 13 and/or 14 do not bind to a non-lactylated protein motif including:

(SEQ ID NO: 15; Unmodified Tau K331la
peptide: 361-375)
CTHVPGGGNKKIETHK.

In some forms, the methods include one or more steps for selecting and screening an antibody produced according to the described methods, for example, in some forms the methods select an antibody for specificity and/or selectivity to bind a lactylated tau polypeptide that includes the sequence of any one of SEQ ID NOs:3, 4-5, 7-8, 10-11, 13-14, or 32-40. In some forms, the methods select an antibody that does not bind to a reference or control protein, such as a tau protein that includes the sequence of any one of SEQ ID NOs:1, 2, 6, 9, 12, or 15.

As discussed elsewhere herein in more details, in non-limiting forms, such molecules can be, for example, a monoclonal antibody, a human antibody, a chimeric antibody or a humanized antibody, or a fragment thereof, and fusion proteins formed therefrom. The antibodies and antigen binding fragments can be monospecific, bispecific, trispecific or multispecific.

Also encompassed are antibodies or their antigen-binding fragments that are conjugated to a diagnostic or therapeutic agent or any other molecule. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance.

Antibodies prepared according to the methods are also provided. For example, antibodies that immunospecifically bind to lactylated tau protein, optionally wherein the lactylated tau protein includes the amino acid sequence of SEQ ID NOs:3-5, 7-8, 10-11, 13-14, or 32-40, are provided. For example, an antibody or antigen binding fragment thereof that selectively binds lactylated tau protein, prepared according to a method including:
(a) immunizing a subject with a recombinant tau protein of including a lactyllysine at one or more positions including K224, K225, K257, K267, K281, K311, K317, K321, and K331 of SEQ ID NO:2; and
(b) isolating an antibody that immuno-specifically binds to the recombinant tau protein from the subject is provided. In some forms, the antibody or antigen binding fragment thereof is immunized with one or more of SEQ ID NOs:3-5, 7-8, 10-11, 13-14, or 32-40. In some forms, the antibody is an intact antibody or functional antibody fragment or fusion protein, for example, selected from Fab fragments, F(ab′)2 fragments, Fab′ fragments, Fv fragments, recombinant IgG (rlgG) fragments, single chain antibody fragments optionally single chain variable fragments (scFv), and single domain antibodies optionally selected from sdAb, sdFv, and nanobody fragments. In some forms, the antibodies are polyclonal antibodies, monoclonal antibodies, multi-specific antibodies, human antibodies, humanized antibodies, synthetic antibodies, chimeric antibodies, camelized antibodies, single chain antibodies, disulfide-linked Fvs (sdFv), intrabodies, and anti-idiotypic (anti-Id) antibodies. In particular, such antibodies include immunoglobulin molecules of any type (e.g., IgG, IgE, IgM, IgD, IgA and IgY), class (e.g., IgG1, IgG2, IgG3, IgG4, IgAQ1 and IgA2) or subclass. Also provided are antibody fragments and antigen binding fragments of the antibodies.
   b. Carrier Proteins

The lactylated tau antigens can be fusion proteins or conjugates further including a carrier protein. Carrier proteins can help elicit a strong immune response against weak antigens. The fusion proteins or conjugates can have a first partner including a lactylated tau antigen fused or conjugated (i) directly to a carrier protein or, (ii) optionally, fused or conjugated to a linker (e.g., peptide linker) that is fused to the carrier protein. The fusion proteins and conjugates can optionally contain a domain that functions to dimerize or multimerize two or more of the molecules. The peptide/polypeptide linker domain can either be a separate domain, or alternatively can be contained within one of one of the other domains (peptide antigen or carrier protein) of the fusion protein. Similarly, the domain that functions to dimerize or multimerize the fusion proteins or conjugates can either be a separate domain, or alternatively can be contained within one of one of the other domains of the fusion protein or conjugate. In some forms, the dimerization/multimerization domain and the peptide/polypeptide linker domain are the same.

In some forms, the fusion proteins and conjugates disclosed herein can be of formula I:

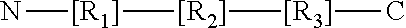

wherein “N” represents the N-terminus of the fusion protein or conjugate, “C” represents the C-terminus of the fusion protein or conjugate, “R₁” is a lactylated tau antigen, “R₂” is an optionally a conjugate or peptide/polypeptide linker domain, and “R₃” is a carrier protein. Alternatively, R₃ may be the lactylated tau antigen and R₁ may be the carrier protein.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

Exemplary carrier proteins include, but are not limited to, Keyhole Limpet Hemocyanin (KLH), Tetanus Toxoid (TT), Diphtheria Toxoid, Bovine Serum Albumin (BSA), Ovalbumin, Mouse Serum Albumin, Rabbit Serum Albumin, Hen Egg Lysozyme (HEL), Mucin, Hapten-Protein Conjugates, etc.

### 3. Nucleic Acid Binders of Lactylated Tau Proteins

Provided are functional nucleic acids that specifically and selectively bind to the described lactylated tau proteins, and which do not bind to non-lactylated tau peptides. Disclosed are functional nucleic acids including, for example, a nucleic acid molecule including an aptamer that selectively and specifically binds to lactylated tau. Aptamers are nucleic acid segments and structures that can bind selectively to particular compounds and classes of compounds.

Aptamers are short single-stranded DNA or RNA oligonucleotides (6˜26 kDa) that fold into well-defined 3D structures that recognize a variety of biological molecules including transmembrane proteins, sugars and nucleic acids with high affinity and specificity (Yu B, et al, Mol Membr Biol., 27(7):286-98 (2010)). The high sequence and conformational diversity of naive aptamer pools (not yet selected against a target) makes the discovery of target binding aptamers highly likely. The selection of aptamers capable of binding a target of interest is called ‘Systematic Evolution of Ligands by Exponential enrichment’ (SELEX). SELEX involves iterative rounds of target binding, partitioning binding from non-binding sequences, and amplification of the enriched binding sequences. Given their unique conformations with ligand-binding characteristics, typical non-immunogenicity and non-toxicity, and ability to be modified for stability in circulation, aptamers are suited to the active targeting of the nucleic acid assemblies described herein. The aptamer may include modified or unmodified DNA or RNA.

Typically, the aptamer adopts a particular secondary- and tertiary-structure fold, typically ranging from about 70 to about 170 contiguous nucleotides, inclusive, such as about 80 nucleotides, or about 100 nucleotides, or any integer in between, such as 80, 81, 82, 83, 84, 85, 86, 87, 88, 89, 90, 91, 92, 93, 94, 95, 96, 97, 98, 99 or 100 nucleotides. Although the reasons for the substantial increase in complexity and information content of the natural aptamer sequences relative to artificial aptamers remains to be proven, this complexity is believed required to form RNA receptors that function with high affinity and selectivity. In some forms, the aptamer includes a nucleotide sequence that forms 2 or 3 hairpin secondary structures.

In some forms, the aptamers are nuclease resistant. In some forms, the aptamer is an RNA aptamer that is 2′-modified (e.g., 2′-fluro and 2′-0-methyl). In some forms, the aptamer (e.g., RNA aptamer) exhibits fluorescence upon binding small molecules. For example, the Spinach and Spinach2 aptamers bind and activate the fluorescence of fluorophores similar to that found in green fluorescent protein, and Broccoli is a 49-nt-long aptamer that exhibits bright green fluorescence upon binding DFHBI or DFHBI-1T (Filonov G S, et al., J Am Chem Soc., 136(46): 16299-308 (2014)).

### 4. Small Molecule Binders

Provided are functional small molecules that specifically and selectively bind to the described lactylated tau proteins, and which do not bind to non-lactylated tau peptides. Typically, the small molecules have a molecular weight less than about 2,000 Da.

Small-molecule binders that selectively recognize and bind lactylated tau or tau fragments comprising one or more lactylated lysine residues are described. Exemplary small molecules include, but are not limited to, peptidomimetics, lactyl-lysine-recognition scaffolds, acyl-lysine binding compounds, or compounds identified through structure-based design or screening approaches (e.g., affinity screening, fragment-based screening, or in silico docking) that preferentially bind lactylated tau relative to non-lactylated tau.

In some forms, small-molecule binders of lactyllysine are used for detection, imaging, diagnostic assays, or therapeutic modulation of tau lactylation, including inhibition of lactylated tau aggregation, promotion of clearance, or interference with pathogenic protein-protein interactions. As described in more detail below, any of the described binders of lactyllysine may be formulated alone or with carriers suitable for delivery to the brain and central nervous system.

### 5. Detectable Compositions

In some forms, the described binding compositions that specifically and selectively recognize and bind to lactylated tau protein motifs include a detectable substance. For example, in some forms, the compositions are configured to be specifically and selectively detected and/or quantified in vitro and/or in vivo.

Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the binding composition or indirectly, such as through an intermediate (such as, for example, a linker known in the art) using techniques known in the art. See, for example, U.S. Pat. No. 4,741,900 for metal ions which can be conjugated to antibodies for use as diagnostics according to the present disclosure. Such diagnosis and detection can be accomplished by coupling the binding composition to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (213Bi), carbon (14C), chromium (51Cr), cobalt (57Co), fluorine (18F), gadolinium (153Gd, 159Gd), gallium (68Ga, 67Ga), germanium (68Ge), holmium (166Ho), indium (11In, 113In, 112In, 111In), iodine (131I, 125I, 123I, 121I), lanthanium (140La), lutetium (177Lu), manganese (54Mn), molybdenum (99Mo), palladium (103Pd), phosphorous (32P), praseodymium (142Pr), promethium (149Pm), rhenium (186Re, 188Re), rhodium (105Rh), ruthemium (97Ru), samarium (153Sm), scandium (47Sc), selenium (75Se), strontium (85Sr), sulfur (35S), technetium (99Tc), thallium (201Ti), tin (113Sn, 117Sn), tritium (3H), xenon (133Xe), ytterbium (169Yb, 175Yb), yttrium (90Y), zinc (65Zn); positron emitting metals using various positron emission tomographies, and nonradioactive paramagnetic metal ions.

In some forms, the detectable compositions can be conjugated to a second antibody to form an antibody heteroconjugate as described by Segal in U.S. Pat. No. 4,676,980. Such heteroconjugate antibodies may additionally bind to haptens (such as fluorescein, etc.), or to cellular markers.

Bispecific and multispecific antibodies that bind the disclosed targets and e.g., a second AD antigen or immune cell antigen are provided.

### 6. Additional Functional Motifs

In some forms, the described binding compositions that specifically and selectively recognize and bind to lactylated tau protein motifs include one or more additional functional motifs. Any of the described molecules can be fused to marker sequences, such as a peptide, to facilitate purification. In some forms, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

In other forms, the described binding compositions that specifically and selectively recognize and bind to lactylated tau protein motifs can be attached to solid supports, which are particularly useful for immunoassays or purification of the target antigen or of other molecules that are capable of binding to target antigen that has been immobilized to the support via binding to an antibody or antigen-binding fragment of the present disclosure. Such solid supports include, but are not limited to, glass, cellulose, polyacrylamide, nylon, polystyrene, polyvinyl chloride or polypropylene.

### C. Nucleic Acids and Host Cells

Nucleic acids and vectors encoding or expressing any of the disclosed polypeptides or nucleic acids are provided. For example, nucleic acids encoding or expressing any or the described protein binders of lactylated tau, and/or nucleic acid binders of lactylated tau are described. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence provided herein.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phospho-triester backbone.

The nucleic acids can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Nucleic acids, such as those encoding or expressing a lactylated tau binder described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, FLAG™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the polypeptides described herein.

### D. Delivery Vehicles

In some forms, compositions that include or encode the described engineered lactylated tau, engineered non-lactylated tau, or selective binders thereof are formulated with, encapsulated within, or conjugated to excipients, carriers, or delivery systems designed to enhance delivery to the brain and/or central nervous system (CNS). Exemplary delivery systems include, but are not limited to, nanoparticles, liposomes, polymeric carriers, lipid-based formulations, viral vectors, extracellular vesicles, or cell-penetrating peptides, including formulations that facilitate transport across the blood-brain barrier (BBB). In some forms, targeting moieties or ligands that promote CNS uptake or cell-type specificity are included. Representative systems and compositions for targeting molecules to the brain and CNS are described, for example, in Sousa, Pharmaceutics 2022 September; 14(9):1835, which is incorporated herein by reference.

The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for inter-tissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some forms the nanoparticles can have a diameter less than 400 nm, less than 300 nm, or less than 200 nm. The range can be between 50 nm and 300 nm.

Thus, in some forms, the delivery vehicles are nanoscale compositions, for example, 10 nm up to, but not including, about 1 micron. However, it will be appreciated that in some forms, and for some uses, the particles can be smaller, or larger (e.g., microparticles, etc.). Although many of the compositions disclosed herein are referred to as nanoparticle or nanocarrier compositions, it will be appreciated that in some forms and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

### 1. Viral Capsid Proteins

In some forms, the delivery vehicle is a viral capsid, or a virus-like particle formed from partly or entirely of a multiplicity of viral capsid proteins. Generally, virus capsids are stable toward thermal denaturation at temperatures up to 80-100° C., chaotropic agents, and to extremes of pH. Exemplary viral-like particles that are stable toward thermal denaturation at temperatures up to 80-100° C., chaotropic agents, and to extremes of pH include bacteriophage capsids and phage particles.

In some forms, the delivery vehicle includes a viral-like particle (VLP), or vesicle, composed of a bacteriophage capsid protein.

The stability of a virus-like particle (VLP) is an important consideration for its use in nanobiotechnology. In some forms, the icosahedral capsid of a bacteriophage is cross-linked by disulfide bonds between coat protein dimers at its 5-fold and quasi-6-fold symmetry axes, providing enhanced stability to VLPs formed from capsid proteins. In some forms, the capsid is a modified capsid, for example, modified by attachment of a peptide, carbohydrate, small molecule or nucleic acid to the viral capsid.

### 2. Polymeric Particles

In some forms, the delivery vehicle is or includes one or more polymers, such as polymeric nanoparticles or microparticles. Exemplary polymers include biocompatible polymers. In some forms, the biocompatible polymer(s) is biodegradable or bioabsorbable. In other forms, the polymer is non-degradable. In some forms, the particles are a mixture of degradable and non-degradable particles.

In some forms, the delivery vehicle is a particles that includes one or more biocompatible polymer(s) including, but not limited to, polyamino acids; cyclodextrin-containing polymers, in particular cationic cyclodextrin-containing polymers, such as those described in U.S. Pat. No. 6,509,323; polymers prepared from lactones such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, poly(valeric acid), and poly-L-glutamic acid; hydroxypropyl methacrylate (HPMA); polyanhydrides; other polyesters; polyorthoesters; poly(ester amides); polyamides; poly(ester ethers); polycarbonates; polyalkylenes such as polyethylene and polypropylene; polyalkylene glycols such as poly(ethylene glycol) (PEG) and polyalkylene oxides (PEO), and block copolymers thereof such as polyoxyalkylene oxide (“PLURONICS®” or block copolymers containing PEG where PEG has a molecular weight of any values within the range of 300 Daltons to 1 MDa); polyalkylene terephthalates such as poly(ethylene terephthalate); ethylene vinyl acetate polymer (EVA); polyvinyl alcohols (PVA); polyvinyl ethers; polyvinyl esters such as poly(vinyl acetate); polyvinyl halides such as poly(vinyl chloride) (PVC), polyvinylpyrrolidone; polysiloxanes; polystyrene (PS); and celluloses including alkyl celluloses, hydroxyalkyl celluloses, cellulose ethers, cellulose esters, nitro celluloses, hydroxypropylcellulose, and carboxymethylcellulose; polymers of acrylic acids including poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; and polyphosphazenes.

Examples of preferred natural polymers include proteins such as albumin, collagen, gelatin and prolamines, for example, zein, and polysaccharides such as alginate. Copolymers of the above, such as random, block, or graft copolymers, or blends of the polymers listed above can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

In some forms, the delivery vehicles are particles modified with one or more surfactants. Examples of surfactants include, but are not limited to, L-α-phosphatidylcholine (PC), 1,2-dipalmitoylphosphatidycholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil, lecithin, oleic acid, and sorbitan trioleate.

In some forms where polyalkylene glycol (e.g., PEG) is used in a composition of polymers to modify the particles, PEG surface density may be controlled by varying the amount of PEG in the polymer composition or by mixing a blend of pegylated polymer component and non-pegylated polymer component. The density of PEG or polyalkylene glycol on the surface of formed particles may be evaluated using several techniques.

In some forms, the delivery vehicles are modified by the addition of one or more polymers to possess a specific ζ-potential. For example, in some forms, the delivery vehicles are modified by the attachment of PEG and/or other polymers to the surface to possess a ζ-potential of between about 20 mV and about −20 mV, preferably between about 10 mV and about −10 mV, more preferably between about 2 mV and about −2 mV.

### 3. Liposomes, Micelles and Lipidic Particles

In some forms, the particles are lipidic particles, such as liposomes, or micelles. Lipidic particles include unilamellar phospholipid vesicles, liposomes, or lipoprotein particles. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes, Chapter 10, 1979.

Formulations of liposomes and methods of making such formulations are well known to one of ordinary skill in the art. Liposomes are formed from commercially available phospholipids supplied by a variety of vendors including Avanti Polar Lipids, Inc. (Birmingham, Ala.).

Suitable methods, materials and lipids for making liposomes are known in the art. Liposome delivery vehicles are commercially available from multiple sources. The liposome may be formed from a single lipid; however, in some forms, the liposome is formed from a combination of more than one lipid. The lipids can be neutral, anionic or cationic at physiologic pH. In some forms, the liposomes incorporate PEG, or PEGylated lipid derivatives. Incorporation of one or more PEGylated lipid derivatives can result in a liposome which displays polyethylene glycol chains on its surface. The resulting liposomes may possess increased stability and circulation time in vivo as compared to liposomes lacking PEG chains on their surfaces. Liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In some forms, the liposomes contain a phosphaditylcholine (PC) head group, and preferably sphingomyelin. In another form, the liposomes contain DPPC. In a further form, the liposomes contain a neutral lipid, preferably 1,2-dioleoylphosphatidylcholine (DOPC).

In certain forms, the liposomes are generated from a single type of phospholipid. In such forms, preferably the phospholipid has a phosphaditylcholine head group, and, most preferably is sphingomyelin. The liposomes may include a sphingomyelin metabolite. Sphingomyelin metabolites used to formulate the liposomes include, without limitation, ceramide, sphingosine, or sphingosine 1-phosphate. The concentration of the sphingomyelin metabolites included in the lipids used to formulate the liposomes can range from about 0.1 mol % to about 10 mol %. Preferably from about 2.0 mol % to about 5.0 mol %, and more preferably can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one form, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one form, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred. The molar ratio of a first phospholipid, such as sphingomyelin, to second lipid can range from about 5:1 to about 1:1 or 3:1 to about 1:1, more preferably from about 1.5:1 to about 1:1, and most preferably, the molar ratio is about 1:1.

### 4. Targeting Moieties and Compositions

Any of the disclosed compositions including a delivery vehicle, such as a nanoparticle, encapsulating an engineered lactylated tau or selective binders thereof or a nucleic acid encoding or expressing these can include a targeting moiety.

It is envisioned that the described selective binders of lactylated tau are effective to arrest, prevent or reverse tauopathies associated with lactylated tau within one or more areas of the central nervous system and/or brain of a subject. Therefore, in some forms, the described compositions include one or more moieties that directs or targets the compositions to the CNS or brain of a subject in vivo. For example, in some forms, the compositions include one or more moieties conjugated to, complexed with or otherwise associated with the engineered selective binders of lactylated tau that facilitate or enhance passage of the compositions across the blood-brain barrier. For example, in some forms, the targeting moiety is a peptide, a lipid, a polymer, a small molecule, a carbohydrate, or combinations thereof.

i. Peptide Targeting Moieties

In some forms, the targeting moiety is a peptide, such as a cell-penetrating peptide (CPP). CPPs are described, for example, in Ghorai, et al., Pharmaceutics 2023, 15(7), 1999, the contents of which are incorporated herein in their entirety. CPPs are known in the art and typically include a 15-25 long amino-acid sequence of amphipathic molecules rich in positively charged amino acids, primarily arginine. Arginine is preferred over lysine owing to the extra H-bond of the guanidium group. Naturally, all characteristic features of CPPs are primarily aimed at improving internalization into the cells. The Pep- and MPG families of small peptides are instances of such amphipathic cell penetrating molecules that can form conjugates with proteins and nucleic acids, respectively, and can aid in obtaining the desired results. CPPs are designed to successfully deliver macromolecules into the cytosol; thus, they are used as delivery systems rather than therapeutic agents. CPPs may be transported directly across the cellular membrane or by entrapment as peptides/cargo within the endosomes. Endocytic pathways usually involve one of the energy-dependent mechanisms such as phagocytosis, caveolae-mediated endocytosis (CvME), clathrin-mediated endocytosis (CME), or cholesterol-dependent endocytosis.

In exemplary forms, CPPS are conjugated directly to a delivery vehicle, such as a nanoparticle, or they may be conjugated via one or more spacers or linkers. Exemplary CCP peptide sequences active in shuttling an associated cargo molecule through the BBB include: ApoE peptide, having an amino acid sequence of LRKLRKRLL (SEQ ID NO:16); ApoB peptide, having an amino acid sequence of SSVIDALQYKLEGTTRLTRKRGLKLATALSLSNKFVEGS (SEQ ID NO:17); hApoE peptide, having an amino acid sequence of LRKLRKRLLR (SEQ ID NO:18); RVG-29 peptide, having an amino acid sequence of YTIWMPENPRPGTPCDIFTNSRGKRASNG (SEQ ID NO:19); TAT peptide, having an amino acid sequence of GGGGYGRKKRRQRRR (SEQ ID NO:20); PepH3 peptide, having an amino acid sequence of AGILKRW (SEQ ID NO:21); Apamin peptide, having an amino acid sequence of H-CNCKAPETALCARRCQQH-NH2 (SEQ ID NO:22); MiniAp-4 peptide, having an amino acid sequence of H-DapKAPETALD-NH2 (SEQ ID NO:23); THRre peptide, having an amino acid sequence of PWVPSWMPPRHT (SEQ ID NO:24); TGN peptide, having an amino acid sequence of TGNYKALHPHNG (SEQ ID NO:25); THR peptide, having an amino acid sequence of THRPPMWSPVWP (SEQ ID NO:26); THRre_2f peptide, having an amino acid sequence of (PWVPSWMPPRHT)₂KKGK(CF)G (SEQ ID NO:27); and K16APoE, having an amino acid sequence of HAYED (SEQ ID NO:28). In other forms, the CCP includes a peptide sequence CNSRLHLRC (SEQ ID NO:29); or CENWWGDVC (SEQ ID NO:30); or WRCVLREGPAGGCAWFNRHL (SEQ ID NO:31). Therefore, in some forms, a delivery vehicle includes or is conjugated to one or more CPP having an amino acid sequence of any one of SEQ ID NOS:15-31.

ii. Formulations for Targeting to the Brain/CNS

In some forms, compositions including or encoding the described engineered lactylated tau or selective binders thereof are formulated with or mixed within a specific excipient or carrier that is designed to enhance delivery to the brain and/or CNS. Systems and compositions for targeting of molecules to the brain are described, for example in Sousa, Pharmaceutics. 2022 September; 14(9): 1835.

### E. Pharmaceutical Compositions

Pharmaceutical compositions containing engineered selective binders of lactylated tau, or nucleic acids encoding, or a delivery vehicle, such as a nanoparticle, encapsulating an engineered selective binder of lactylated tau or a nucleic acid encoding an engineered selective binder of lactylated tau, are also described.

In some forms, the pharmaceutical compositions include one or more of a pharmaceutically acceptable buffer, carrier, diluent or excipients.

The term “pharmaceutically acceptable carrier” describes a pharmaceutically acceptable material, or composition, that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some forms, pharmaceutical compositions include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. The pharmaceutical compositions can be formulated for delivery via any route of administration. The term “route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Biomarkers of Tau Lactylation and Uses Thereof

As described in the Examples, it has been established that lysine lactylation in tau protein plays a significant role associated with tauopathies such as AD, and tau lactylation therefore represents a biomarker for the study of tauopathies.

The observed upregulation of lactate signature genes and lactylation of tau correlates with increased tau phosphorylation and cleavage, highlighting lactate as a mediator of tau pathophysiology. These results suggest that lactylation not only serves as a biomarker for AD but also potentially contributes to tau's pathogenic features, such as its aggregation and impaired clearance. Furthermore, in vitro experiments indicate that lactate directly induces tau lactylation and affects its turnover, providing new insights into the metabolic regulation of tau and offering potential therapeutic avenues for targeting lactate metabolism in AD. As introduced above, lactylation of tau protein in the CNS or brain can be used as a biomarker for AD diagnosis and/or other diseases and disorders characterized by aberrant or increased levels of tau lactylation, identifying the corresponding subject as a target for treatment, and guiding such treatment selection. Lactylation of tau protein in the CNS or brain can also be used to increase the positive predictive value of current screening modalities and for selection and monitoring the efficacy of treatment regimens. Such compositions and methods are disclosed herein.

The methods typically include detecting lactylation of tau protein in the CNS or brain sample from a subject. For example, cells in a biological sample can be suspected of having increased lactylation of tau protein in the CNS or brain. Aberrant or increased levels of tau lactylation can be characterized by the simultaneous activation of multiple pathways regulating cell proliferation and invasion. In some forms, aberrant or increased levels of tau lactylation activity includes over or unregulated expression of tau lactylation, hyper-phosphorylation of tau lactylation substrates including, but not limited to, K224, K225, K257, K267, K281, K311, K317, K331, and K321 of SEQ ID NO:2, or a combination thereof.

For example, in some forms, a biomarker for the detection, monitoring and determining the status of a tauopathy, such as AD, in a subject includes a lactylated tau protein within a biological sample. Exemplary lactylated tau proteins include proteins having an amino acid sequence set forth in any one of SEQ ID NOs:3, and 32-40. Therefore, in some forms, the methods detect a protein including an amino acid sequence set forth in any one of SEQ ID NOs:3, and 32-40.

In some forms, a biomarker for the detection, monitoring and determining the status of a tauopathy, such as AD, in a subject includes a lactylated tau protein within a biological sample having lactylated lysine at position 331 of SEQ ID NO:2. For example, in some forms, a biomarker for the detection, monitoring and determining the status of a tauopathy, such as AD, in a subject includes a lactylated tau protein having an amino acid sequence set forth in SEQ ID NO:36.

In some forms, the cells are suspected or known to be AD cells. Any of the methods can further include detecting one or more AD antigen(s) or other disease markers to further characterize the sample. For example, overexpression of tau lactylation has been detected in AD that may serve as additional diagnostic markers of aberrant or increased levels of tau lactylation in disease.

In some forms, the methods diagnose pathological tau formation in a subject by quantitation of lactylated tau proteins in a sample, such as CSF, from the subject. While it is understood that a baseline or threshold amount of lactylated tau protein is present in a sample, such as CSF, from a healthy, non-diseased subject, it has been established that an increase in the amount of lactylated tau protein is associated with pathological tau formation. Therefore, in some forms, the methods measure and compare lactylated tau protein in a sample, such as CSF, from a subject and compare the amount of lactylated tau protein in the sample, with that of a control, such as CSF from a non-diseased or healthy subject that does not have pathological tau aggregation. In some forms, the quantitation and comparison is carried out for all potential lactyllysines in the tau protein in each sample. In other forms, the quantitation and comparison is carried out for one or more potential lactyllysines in the tau protein in each sample. For example, in some forms, the methods measure and compare tau lactylation at one or more lysine residues including, but not limited to, K224, K225, K257, K267, K281, K311, K317, K331, and K321 of SEQ ID NO:2. In some forms, the methods measure and compare tau lactylation at one or more lysine residues including, but not limited to, K281 and K331, of SEQ ID NO:2. Differences in the amount of lactyllysine over two or more different samples can be, for example, from 1% to 100%, inclusive, such as 10%, 20%, 30%, 40%, 50%, 60%, 70%, 80%, 90% or more than 100%, such as 200%, 300%, 400%, 500%, 600%, 700%, 800% or 900% or more than 900% increase in lactyllysine relative to a control.

### A. Sources of the Biomarkers

The disclosed biomarkers are lactylated tau proteins. Some forms provide these biomolecules in isolated form such as in a biological sample. The preferred biological source for detection of the biomarkers is tissue (e.g., tissue suspected of being associated with a tauopathy) including biopsy material from the CNS or brain, or cells thereof. In some forms, the cells are blood cells such as white blood cells such as immune cells, or plasma cells.

In some forms, intact cells are subjected to biomarker detection. For example, a sample may be obtained and processed using well-known and routine clinical methods. In some aspects, the biological sample includes a plurality of cells. In certain aspects, the biological sample includes fresh or frozen tissue. In specific aspects, the biological sample includes formalin fixed, paraffin embedded tissue. In some forms, the cells are permeabilized. In some forms, a cell lysate or homogenate is subjected to biomarker detection.

### B. Methods of Detecting Biomarkers

The disclosed lactylated tau biomarkers can be detected by any suitable means. In some forms, the biomarkers are detected using a binding molecule such as an antibody. In addition to immunoassay-based methods, non-immunoassay detection methods for lactylated tau are also contemplated. In some forms, the methods include mass spectrometry-based detection methods, such as LC-MS/MS, targeted MS (e.g., PRM, SRM/MRM), and quantitative proteomics approaches for detection and quantification of lactylated tau and specific lactylated lysine residues.

In some forms, the methods include chemical labeling or enrichment strategies coupled to MS, including affinity capture of lactylated peptides or derivatization approaches.

In some forms, the methods include chromatographic methods (e.g., HPLC or UPLC) coupled with MS or other detectors.

In some forms, the methods include imaging and spectroscopy-based methods, including fluorescent, luminescent, or radiolabeled probes that selectively bind lactylated tau.

In some forms, the methods include biosensor-based approaches, including aptamer-based, electrochemical, or surface plasmon resonance (SPR)-based detection of lactylated tau.

In some forms, the methods include the described detection methods are applied to brain tissue, cerebrospinal fluid, blood, plasma, serum, or other biological samples; the methods may be used alone or in combination with immunoassays for diagnostic, prognostic, or research purposes.

In some forms, the disclosed lactylated tau biomarkers are detected and quantitated using one or more of the tau binding molecules, such as antibodies or other antigen binding molecules provided herein. The biomarkers can be detected by any suitable method utilizing the provided antibodies. In preferred forms, the biomarkers are detected and/or measured by an immunoassay. Immunoassays utilize biospecific capture reagents, such as antibodies, to capture or locate the biomarkers.

The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label (also referred to as a detectable substance or reporter), wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Traditional immunoassays including, for example, sandwich immunoassays including ELISA or fluorescence-based immunoassays, as well as other enzyme immunoassays can be used for detecting the biomarkers. In other forms, the detection of the biomarker is carried out on slides of test material (e.g., immunohistochemistry), Western blotting, surface plasmon resonance (e.g., Biacore), or by flow cytometry (FACS) analysis).

Other specific examples include, but are not limited to, enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, and immuno-chromatography assays. Most assays now use nonradioactive labels. Enzyme immunoassays (enzyme-linked immunosorbent assays, or ELISA; immunometric assays) can use enzymes as labels, such as, for example, horseradish peroxidase or alkaline phosphatase. Chemiluminescent immunoassays (CIA) can use luminol. Fluorimetric immunoassays (FIA) use fluorescent compounds (e.g., fluorescein) as labels.

The assays can be homogenous or heterogeneous assays, competitive and noncompetitive assays.

In some forms, ELISA is used to identify and quantify the presence of lactylated tau. There are four main kinds of ELISA: sandwich, competitive, direct, and indirect assays. These methods differ in how the antibody or antigen is attached to the solid plate, and how the signal is detected. For example, in a sandwich ELISA, for example, an antibody is immobilized on a plate. The sample containing the target antigen is added, which binds to the antibody and so is immobilized on the plate. Next, a second type of antibody is added, which also binds to the target antigen on the plate, forming a ‘sandwich’ with the target antigen in the middle. The second antibody is linked to an enzyme, called a reporter enzyme, which allows the binding reaction to be measured by creating a color signal. To create this signal, first any unbound antibody is washed away, and a colorimetric substrate is added. The enzyme catalyzes a reaction of the substrate, creating a color change. A stronger color signal indicates more target antigen is present. An example of this is a home pregnancy test. In some forms, the first or second antibody is one of the disclosed antibodies, and the other antibody is one that detects the protein of interest or the biomarker (e.g., lactylation of tau), but may or may not detect its phosphorylated state, and thus may target a different antigen of the protein (e.g., a non-lactylated antigen).

In some forms, the assay is in the form of a sandwich assay, which is a noncompetitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person.

Immunohistochemistry (IHC) is a process of localizing antigens (e.g., proteins) in tissue utilizing antigen-specific antibodies. The antigen-binding antibody can be conjugated or fused to a tag that allows its detection, e.g., via visualization. In some forms, the tag is an enzyme that can catalyze a color-producing reaction, such as alkaline phosphatase or horseradish peroxidase. The enzyme can be fused to the antibody or non-covalently bound, e.g., using a biotin-avidin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag.

Quantitative immunochemical techniques can also be used. For example, the Quantitative Tissue Biomarker Platform from HistoRx and/or measuring immunofluorescence level(s) can be used to quantify levels of biomarkers.

Western blotting can be used to identify and/or quantify the presence of lactylated tau in a biological sample, and can be quantitative or qualitative. A typical Western blotting procedure includes the steps of immunoprecipitating a target protein from a lysate of cells expressing the protein, performing an SDS-PAGE with said protein, transferring the protein to a nitrocellulose membrane, incubating the nitrocellulose membrane with said antibody, detecting said antibody with a secondary antibody conjugated to a fluorescent or chromogenic compound (e.g., peroxidases such as horseradish peroxidase (HRP), alkaline phosphatase (AP), IRDye near-infrared (NIR) fluorescent dyes), and quantifying the respective signal of said compound (e.g., fluorescence, luminescence, chromogenic enzyme substrate).

The ratio of two signals generated by Western blotting employing the same antibody but two different samples can be calculated, thereby determining how much more/less (fold-change) of the biomarker is present in one sample compared to another.

In some forms, the methods provided herein involve determining the presence, absence, and/or concentration of lactylated tau biomarkers in a cell, and/or the number of biomarker positive cells in sample by fluorescence activated cell sorting (FACS) using a flow cytometry device (e.g., Beckman Coulter Z2 Coulter Counter, Beckman Coulter Inc.). In some forms, a FACs-based method include the step of preparing the output composition for detection by flow cytometry before the biomarker can be detected. For example, the output composition can be incubated with a fluorescently labeled antibody that is specific for one or more biomarkers, and then the sample can be analyzed using a flow cytometer. In some forms, the cells are permeabilized to facilitate antibody access to intracellular lactylated tau biomarkers. In flow cytometry, cells bound by fluorescently labeled affinity reagents are carried in a fluidic stream, are separated based on size and/or fluorescent signal and are subsequently analyzed and counted using a FACS software program (e.g., FlowJo software). The number or approximate number of cells can be determined by detection of the fluorescent signal, which optionally can be determined or processed by the FACS software program to provide the total or approximate number of particles in the output composition.

In some forms, a sample, such as a biological sample derived from the CNS or brain is analyzed by means of a biochip. Biochips generally include solid substrates and have a generally planar surface, to which a capture reagent (also called an adsorbent or affinity reagent) is attached. Frequently, the surface of a biochip includes a plurality of addressable locations, each of which has the capture reagent bound there.

In some forms, the biochips are protein biochips. Protein biochips are adapted for the capture of polypeptides. Many protein biochips are described in the art. These include; for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden).

Photonic biosensors can be used in label-free assays. For example, photonic biosensors combine photonic sensing with bio recognition technology to create label-free testing on-chip. Instead of moving electrons around on silicon chips, light is moved around on silicon chips via waveguides. This technology has allowed the development of miniature lab-on-a-chip label-free immunoassay (LFIA) devices. These devices are functionalized with capture antibodies and have a resonance condition of light. This resonance wavelength will be shifted by a reaction between the capture antibody and the target antigen due to the change in refractive index. Measuring the shift in resonance wavelength provides a readout of a binding event. Label-free assays therefore enable the detection of antigen-antibody binding without the use of an additional label, resulting in increased assay sensitivity and decreased working time.

In some forms, the methods implement additional biochemical, cellular, and in vivo assays for detecting, quantifying, or modulating lactylated tau.

In some forms, the methods implement cell-based assays assessing tau lactylation, aggregation, turnover, phosphorylation, ubiquitination, cleavage, or subcellular localization in response to metabolic, genetic, or pharmacologic perturbations.

In some forms, the methods implement protein-protein interaction assays (e.g., pull-down assays, proximity ligation assays, FRET/BRET, or crosslinking-based assays) to evaluate interactions involving lactylated tau.

In some forms, the methods implement aggregation and seeding assays, including in vitro fibrillization, biosensor cell assays, or tau propagation models.

In some forms, the methods implement clearance and degradation assays, including autophagy- or proteasome-based assays.

In some forms, the methods implement animal-based assays, including behavioral, biochemical, histological, or imaging readouts in tauopathy models.

In some forms, the methods implement screening assays for identifying compounds or biological agents that modulate tau lactylation or its downstream effects.

These assays may be used for diagnostic, prognostic, therapeutic screening, or mechanistic studies, and may be performed alone or in combination with other detection or treatment methods described herein. Any of the detection methodologies can be multiplexed to detect two, three, four, or more biomarkers. In a particular forms, the methodology is or includes a multiplex immunoassay utilizing Luminex microbead technology.

### C. Diagnosis

Methods of diagnosing a disease or disorder associated with elevated lactylation of tau in a subject are also provided. As set forth in the Examples, it has been established that lactate is an inducer of tau lactylation, phosphorylation, and cleavage, and that the acetyl-transferase p300 mediates the transfer of lactyl-CoA to lysine residues in tau, to produce lactylated tau. It has also been established that elevated lactylation of tau, particularly at the lysine residue K331, is associated with diseases and disorders, including AD. Particularly, in AD a progressive buildup of lactylated tau at K331 in the brain based on Braak stages has been identified.

Therefore, in some forms, methods for diagnosing the presence, severity, progression, regression or absence of a disease or disorder in a subject include detecting or quantifying the amount of lactylated tau in a sample from the subject as compared to a control sample, such as a sample from a healthy subject. In some forms, the methods identify and/or quantify lactylated lysine residues at one or more sites in tau protein in a sample from a subject. For example, in some forms, the methods detect or quantify lactylated lysine at position K311 of a tau protein set forth in SEQ ID NO:2, for example, as set forth in SEQ ID NO:3 or 36.

In some forms, the location, presence and/or quantity of the disclosed lactylated tau biomarkers are used for diagnosis of a tauopathy in a subject. In some forms, the increase in lactylated tau in a subject, for example, an increase in the relative amount of lactylation of tau in a first sample taken at a first time point and a second sample taken at a second time point after the first time point, represents a progression in a disease or disorder associated with tau lactylation, such as AD. For example, in some forms, an increase in the relative amount of lactylation of tau in a sample from a subject with early mild cognitive impairment (MCI) is indicative of progression to a potentially more severe neurodegenerative disease stage. Therefore, in some forms, the methods identify lactylated tau to predict the onset of a disease stage in a subject, or to monitor the progression of a disease, for example in response to treatment or therapy. For example, in some forms, the methods identify or quantify lactyalted tau in a sample from as subject, such as a CSF sample, prior to, during and/or after treatment of the subject. When the methods include more than a single sampling and detection of lactyalted tau in the same subject the methods can include one or more steps to compare the amount of lactylated tau in each of the samples and/or a control sample.

It has been established that lactylated tau is also present in the brain if healthy (control) subjects. Therefore, in some forms, the amount of tau lactylation informs the disease state of a subject. For example, in some forms, the relative amount or incidence of lactylation, such as the amount or incidence of lactyllysine at one or more specific lysine residues in tau from the brain of a subject having a disease or disorder is indicative of a disease or disorder in a subject. It has been established that the presence and amount of lactyllysine at position K311 of SEQ ID NO:2 (e.g., as set forth in SEQ ID NO:36) is indicative of a disease or disorder as compared to a control protein.

As discussed herein, lactylated tau biomarkers can be detected by any suitable method. The methods can include single markers, or combinations of markers.

### 1. Single Markers

In some forms, the biomarkers are used in diagnostic tests to assess AD and/or other tau lactylation-related disease and disorder status in a subject, e.g., to distinguish between normal cells and diseased cells, and disease status. For example, disease status includes, without limitation, the presence or absence of disease (e.g., AD v. non-AD), characterization of cells including AD cells (e.g., level of tau lactylation), the risk of developing disease, the stage of the disease (e.g., non-invasive or early-stage AD v. invasive or metastatic AD), the progress of disease (e.g., progress of disease or remission of disease over time) and the effectiveness or response to treatment of disease. Based on this status, further procedures may be indicated, including additional diagnostic tests or therapeutic procedures or regimens. Representative ADs and therapies are discussed in more detail below.

The biomarkers discussed herein can be present and/or expressed in AD including but not limited to MTC, and, therefore, each is individually useful in aiding in the determination of AD and/or other tau lactylation-related diseases and disorders. The method involves, first, measuring the selected biomarker in a subject sample using the methods described herein, and, second, comparing the measurement with a diagnostic amount or cut-off that distinguishes a positive AD and/or other tau lactylation-related disease and disorder status from a negative AD and/or other tau lactylation-related disease and disorder status. The diagnostic amount represents a measured amount of a biomarker above which a subject is classified as having a particular status. For example, because the biomarker is up-regulated compared to normal during AD and/or other tau lactylation-related diseases and disorders, then a measured amount above the diagnostic cutoff provides a diagnosis or status of the AD and/or other tau lactylation-related diseases and disorders. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different AD statuses and drawing the cut-off to suit the diagnostician's desired levels of specificity and sensitivity.

### 2. Combinations of Markers

In some forms, detecting and/or quantifying one or more diseases requires one or more further biomarkers in addition to lactylated tau. While individual biomarkers, such as lactylated tau are useful diagnostic biomarkers, a combination of biomarkers may provide greater predictive value of a particular status than single biomarkers alone. Specifically, the detection of a plurality of biomarkers in a sample can increase the sensitivity and/or specificity of the test. Thus, in one form, two or more, three or more, four or more or five or more biomarkers can be detected and used to assess the status of AD and/or other tau lactylation-related disease and disorder in a subject.

### D. Determining Risk of Developing Disease

In some forms, the biomarkers are used in methods for determining the risk of developing disease in a subject are also provided. Biomarker amounts or patterns can be characteristic of various risk states, e.g., high, medium, or low. The risk of developing a disease is determined by measuring the relevant biomarker or biomarkers and then either submitting them to a classification algorithm or comparing them with a reference amount and/or pattern of biomarkers that is associated with the particular risk level.

### E. Determining Stage of Disease

In some forms, detecting and/or quantifying the described lactylated tau biomarkers provide methods for determining the stage of disease in a subject. In some forms, a disease, such as a tauopathy, such as AD, includes multiple stages of severity and/or progression. Therefore, in some forms, each stage of a disease, such as a tauopathy, such as AD, can be associated and/or correlated with a characteristic amount of a lactylated tau biomarker or relative amounts of a set of biomarkers (a pattern). In some forms, methods to determine, monitor and/or predict each stage of a disease, such as a tauopathy, such as AD, include one or more steps for measuring the presence, amount or location of lactylated tau biomarker alone, or together with one or more additional biomarkers and then either submitting them to a classification algorithm or comparing them with a reference amount and/or pattern of biomarkers that is associated with the particular stage.

### F. Determining Course (Progression/Remission) of Disease

In some forms, detecting and/or quantifying the described lactylated tau biomarkers is used in methods for determining the course of disease in a subject. Disease course refers to changes in disease status over time, including disease progression (worsening) and disease regression (improvement). Over time, the amounts or relative amounts (e.g., the pattern) of the biomarkers changes. This method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

### G. Subject Management

In some forms, detecting and/or quantifying the described lactylated tau biomarkers is used in methods for managing subject treatment based on the status. Such management includes the actions of the physician or clinician subsequent to determining AD and/or other tau lactylation-related disease and disorder status. For example, if a physician makes a diagnosis of AD and/or other tau lactylation-related disease and disorder, then a certain regime of treatment, such as prescription or administration of therapy, including, but not limited to administration of the compositions discussed in more detail below, might follow. Alternatively, a diagnosis of non-AD might be followed with further testing to determine a specific disease that the patient might be suffering from. Also, if the diagnostic test gives an inconclusive result on AD and/or other tau lactylation-related disease and disorder, further tests may be required.

In some forms, detecting and/or quantifying the described lactylated tau biomarkers is used in methods for selecting a subject for treatment for AD and/or other tau lactylation-related disease and disorder by detecting the presence or quantity of one or more biomarkers provided herein in a sample from a subject suspected of having AD and/or other tau lactylation-related disease and disorder, comparing the levels of biomarker in the sample to a predetermined standard, wherein the patient is selected for treatment for AD and/or other tau lactylation-related disease and disorder if certain biomarkers or levels of biomarkers are detected in the sample. Such treatments can be those known to be effective and/or preferred for treating subjects with a tau lactylation-positive conditions.

In some forms, detecting and/or quantifying the described lactylated tau biomarkers is used in methods for identifying a subject as not having a tau lactylation-related disease and disorder, when the test is negative. Thus, although the subject may have AD or another diseases or disorder, the subject can be identified as negative for tau lactylation diseases and disorders. Such forms may lead to selection of alternative treatments and may avoid treatments known to be effective or preferred for treating subjects with tau lactylation conditions, and/or may include treatments that are known not to be effective and/or preferred for treating subjects with conditions associated with aberrant or increased lactylation of tau.

Additional forms relate to the communication of assay results or diagnoses or both to technicians, physicians or patients, for example. In certain forms, computers will be used to communicate assay results or diagnoses or both to interested parties, e.g., physicians and their patients. In some forms, the assays will be performed or the assay results analyzed in a country or jurisdiction which differs from the country or jurisdiction to which the results or diagnoses are communicated.

In a preferred form a diagnosis based on the presence or absence in a test subject of any of the disclosed biomarkers is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain forms, the message containing results of a diagnostic test may be generated and delivered automatically to the subject using a combination of computer hardware and software which will be familiar to artisans skilled in telecommunications. In certain forms all or some of the method steps, including the assaying of samples, diagnosing of diseases, and communicating of assay results or diagnoses, may be carried out in diverse (e.g., foreign) jurisdictions.

### H. Biomarkers in Screening Assays

In some forms, detecting and/or quantifying the described lactylated tau biomarkers is used in methods to screen for compounds that modulate the expression of the biomarkers in vitro or in vivo, which compounds in turn may be useful in treating or preventing tau lactylation-related disease and disorder in patients. Compounds suitable for therapeutic testing may be screened initially by identifying compounds which reduce the presence of one or more biomarkers in the tau lactylation-related disease and disordered cells.

Test compounds capable of modulating the presence and/or expression of any of the biomarkers in tau lactylation-related disease and disorder may be administered to patients who are suffering from or are at risk of developing AD. For example, the administration of a test compound that decreases the activity of a particular biomarker may decrease the risk of tau lactylation-related disease and disorder in a patient if the increased activity of the biomarker is responsible or indicative, at least in part, for the onset of the tau lactylation-related disease and disorder.

At the clinical level, screening a test compound includes obtaining samples from test subjects before and after the subjects have been exposed to a test compound. The levels in the samples of one or more of the biomarkers can be measured and analyzed to determine whether the levels of the biomarkers change after exposure to a test compound. The samples can be analyzed by any appropriate means known to one of skill in the art including e.g., by the means described herein. In a further form, the changes in the level of expression of one or more of the biomarkers can be measured using in vitro methods and materials. For example, human tissue cultured cells which express, or are capable of expressing, one or more of the biomarkers may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with tauopathies and/or other tau lactylation-related disease and disorder, test compounds will be screened for their ability to slow or stop the progression of the disease.

### I. Assessing the Effectiveness of Treatment or Risk

In some forms, detecting and/or quantifying the described lactylated tau biomarkers is used in methods for determining the course of diseases and disorders in a subject. Disease course refers to changes in disease status over time, including disease progression (worsening) and disease regression (improvement). Over time, the amounts or relative amounts (e.g., the pattern) of the biomarkers changes. Accordingly, this method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

In yet another example, detecting and/or quantifying the described lactylated tau biomarkers is used in methods for biomarkers can be used in studies to determine if the subject is at risk for developing AD and/or other tau lactylation-related disease and disorder.

### IV. Methods of Treatment

Methods of treatment of a disease or disorder associated with lactylated tau protein in a subject in need thereof are also provided. For example, any of the disclosed methods for detecting and/or quantifying the described lactylated tau biomarkers may be coupled to a method of treating a subject in need thereof. Thus, any of the disclosed methods can further include treating a subject identified according to the described methods as having an increased tau lactylation as compared to a normal control subject.

In some forms, the described methods include one or more steps for treating a subject identified as having increased tau lactylation as compared to a normal control subject for a disease or disorder associated with increased tau lactylation. In some forms, the methods include treating a subject with a treatment known to be effective and/or preferred for treating subjects with one or more conditions associated with increased tau lactylation.

In some forms, the method includes selecting a subject as having, or as being at increased risk of having a disease or disorder associated with increased tau lactylation and selecting a suitable treatment or regimen for the subject. For example, in some forms, the methods further include selecting a treatment or regimen that is likely to be effective and/or preferred for treating subjects with increased tau lactylation conditions and/or selecting against treatments known to be effective or preferred for treating subjects that do not exhibit increased tau lactylation. In some forms, the methods further include treating the subject with a selected treatment or regimen.

In certain forms, the described compositions are administered to a subject systemically, locally, or regionally. In some forms, the compositions are taken orally, injected, topically applied, or otherwise administered directly into the vasculature or onto vascular tissue at or adjacent to a site of a disease or disorder, such as the CNS and/or the brain. Typically, local administration causes an increased localized concentration of the compositions, which is greater than that which can be achieved by systemic administration.

As set forth in the Examples, it has been established that lactylation not only serves as a biomarker for diseases and disorders such as AD, but also potentially contributes to tau's pathogenic features, such as its aggregation and impaired clearance. Particularly, in AD a progressive buildup of lactylated tau at K331 in the brain based on Braak stages has been identified.

It may be that peptides including a defined lactyllysine that is present in tau, such as lactyllysine at position 281 of SEQ ID NO:2, can reduce the level, formation, or production of neurofibrillary tangles (NFTs) in the brain.

The reduction of pathological tau formation by administration of a lactylated K281 tau peptide is believed to occur through one or more non-mutually exclusive mechanisms, and the invention is not limited to any single mechanism of action.

In some forms, the lactylated K281 peptide may act as a competitive or decoy substrate, interacting with tau-binding partners, aggregation-prone interfaces, or seeding assemblies, thereby interfering with tau-tau interactions required for pathological aggregation and propagation.

In other forms, the lactylated peptide may modulate tau clearance pathways, promote recognition by cellular quality-control machinery, or alter the local biochemical environment to reduce aggregation, seeding, or stability of pathogenic tau species. The presence of the lactyl modification may also influence protein-protein interactions or enzymatic processes involved in tau turnover.

Accordingly, the observed reduction in pathological tau is consistent with a functional interference effect, which may include competitive inhibition, sequestration, altered aggregation dynamics, or enhanced clearance, without being limited to a single defined mechanism. The precise molecular mechanism may vary depending on context.

Methods including administration of a peptides including a defined lactyllysine that is present in tau to reduce the amount or distribution of lactylated tau in the brain of a subject in vivo are provided. It is believed that the compositions disclosed herein are effective to treat a number of tauopathies, as discussed in more detail below.

The disclosed compositions and methods can be used to limit the progression of, reduce, delay, or inhibit the level, formation, or production of diseases and disorders associated with lactylated tau in a subject over time; limit the progression of, reduce, delay, or inhibit the expression of lactylated tau in a subject over time, limit the progression of, reduce, delay, or inhibit the level of neurofibrillary tangles (NFTs) in a subject; increase the relative amount of non-lactylated tau in the brain of a subject; reduce neurodegeneration in the brain of a subject; and combinations thereof. In some forms, the compositions are administered in an effective amount to reduce the expression or accumulation of lactylated tau including lactyllysine at position K311 of tau 441 protein in a subject.

In other forms, the subject has been diagnosed or is likely to be diagnosed with a tauopathy or a disorder, condition, symptom or comorbidity thereof. The disclosed methods typically include administering to the subject an effective amount of the disclosed compositions including a lactylated tau peptide, such as CQIINK(K-lactyl)LDLSN (SEQ ID NO:7), or CINK(K-lactyl)LDLSNVQSKC (SEQ ID NO:8). to limit the progression of, reduce, delay, or inhibit the level, formation, or production of neurofibrillary tangles (NFTs) in subject over time; limit the progression of, reduce, delay, or inhibit the expression of lactylated tau in a subject over time, limit the progression of, reduce, delay, or inhibit the level of neurodegeneration in the brain of a subject; increase the relative amount of non-lactylated tau in the brain of a subject; or a combinations thereof.

As discussed in more detail below, the disclosed compositions and methods can be used to treat one or more tauopathies or disorders, conditions, symptoms or comorbidities thereof. In preferred forms, the subject is a human subject. The subject can be male or female.

Although some tauopathies occur with higher frequency in adult or elderly subjects relative to younger age groups, however other age groups can also be affected by these diseases and conditions. Therefore, newborns, infants, children, adolescents, adults, and elderly can be treated for a tauopathy using the compositions and methods disclosed herein. In some preferred forms the subject is elderly.

In some forms, the composition is administered in an effective amount to treat one or more one or more symptoms or comorbidities of a tauopathy, or a disorder or condition related thereto. The effects of the treatment can be measured relative to a control. Suitable controls are known or can be determined by one of skill in the art. For example, in some forms, the disclosed compositions and methods limit the progression of, reduce, delay, or inhibit the level, formation, or production of neurofibrillary tangles (NFTs) in subject over time; limit the progression of, reduce, delay, or inhibit the expression of lactylated tau in a subject over time, limit the progression of, reduce, delay, or inhibit the level of neurodegeneration in the brain of a subject; increase the relative amount of non-lactylated tau in the brain of a subject; or combinations thereof in a treated subject relative to an untreated subject.

In other forms, the compositions and methods limit the progression of, reduce, decrease, or ameliorate one or more symptoms, characteristics or comorbidities of a tauopathy; reverse the progression of one or more symptoms, characteristics or comorbidities of a tauopathy; halt the progression of one or more symptoms, characteristics or comorbidities of a tauopathy; limit the progression of the occurrence of one or more symptoms, characteristics or comorbidities of a tauopathy; inhibit the rate of development of one or more symptoms, characteristics or comorbidities or combinations thereof of a tauopathy in a treated subject relative to an untreated subject. The untreated subject can be the treated subject prior to initiation of treatment, or a matched subject not receiving the treatment.

Suitable compositions for use with the disclosed methods are discussed in more detail below and typically include a lactylated tau peptide, for example, CQIINK(K-lactyl)LDLSN (SEQ ID NO:7) or CINK(K-lactyl)LDLSNVQSKC (SEQ ID NO:8).

### A. Diseases, Disorders, and Conditions to be Treated

As discussed above, the compositions disclosed herein can be used to prevent, reduce, delay, or inhibit the formation or limit the progression of, reduce, delay, or inhibit the level, formation, or production of diseases and disorders associated with lactylated tau in a subject over time; limit the progression of, reduce, delay, or inhibit the amount of lactylated tau in the brain and CNS of a subject over time, limit the progression of, reduce, delay, or inhibit the level of neurofibrillary tangles (NFTs) in a subject; increase the relative amount of non-lactylated tau in the brain of a subject; reduce neurodegeneration in the brain of a subject; and combinations thereof. In some forms, the compositions are administered in an effective amount to reduce the expression or accumulation of lactylated tau including lactyllysine at position K311 of tau 441 protein in a subject. The compositions are particularly useful for treating a subject with, or likely to develop, a tauopathy. In some forms, the described compositions and methods treat, prevent or ameliorate one or more symptoms of Alzheimer's disease (AD) in a subject in need thereof.

### 1. Tauopathies

In some forms, the disclosed compositions and methods are used to treat or prevent diseases characterized by increased tau lactylation, especially at position K331 of tau 441, tau expression, increased tau phosphorylation, or pathologies associated with the aggregation of tau protein in the brain.

Tauopathies are neurodegenerative diseases where tau aggregates into neurofibrillary tangles (NFTs). Exemplary tauopathies include AD, corticobasal degeneration, frontotemporal dementia, frontotemporal lobar degeneration, progressive supranuclear palsy, and Pick's disease. Huntington's Disease also displays an increased amount of tau, including rod-like deposits. Tau can go through a number of post-translational modifications (PTMs) including phosphorylation, glycation, nitration, O-GlcNAcylation, acetylation, oxidation, ubiquitination, sumoylation, and methylation. Tau's binding affinity to microtubules is regulated by these PTMs. There are high levels of ubiquitinated tau in AD and other tauopathies, and the tau function is impaired and aggregation promoted by lysine acetylation and other PTMs. In transgenic mouse models of tauopathies it is possible to detect acetylation of K280, however, this is not possible in control mouse brains. Of the amino acids in tau, around 20% could potentially become phosphorylated. The microtubule-binding domain of tau is usually positively charged and, as such, attracts the negatively charged microtubules. It loses its positive charge when this domain becomes hyperphosphorylated and tau dissociates from the microtubules. Tau is then no longer able to assemble or stabilize microtubules. It is possible that subsequent neurodegeneration is a result of a loss in microtubule function, hyperphosphorylated tau being toxic to neurons, or a combination of these effects. Tau dimers can be formed through the combination of two hyperphosphorylated tau monomers. Known as dimerization, these interactions between hexapeptides in repeats 2 and 3 can lead to subsequent oligomerization. Following this, oligomers aggregate into Paired Helical Filaments (PHFs), which have a twisted double-helical ribbon structure. PHFs are more negatively charged than monomeric tau when contained within PHFs. It is, therefore, not able to bind tubulin or stabilize microtubules as effectively. If the microtubules are not stabilized then the microtubule network decays and neurons cease to function. Neurofibrillary Tangles (NFT) are filamentous, insoluble aggregate of tau. NFTs have an increased beta-sheet structure and include aggregated PHFs. There is an association between dementia severity and the degree of NFT deposition in the brain as well as an association with neuron death. Neuropil threads and neuritic plaques are two other forms of tau aggregates in AD patients, which also induce neuron degeneration. Research suggests, however, that filamentous and fibrillary tau could have some neuroprotective effects and that the most toxic form of tau is soluble, hyperphosphorylated tau oligomers.

For example, a method of treating a disease or disorder characterized by increased tau lactylation, especially at position K331 of Tau 441, tau expression, increased tau phosphorylation, or pathologies associated with the aggregation of tau protein in the brain can include administering to a subject in need thereof a composition including an effective amount of a lactylated tau protein, to reduce, delay, or inhibit tau lactylation, or development of neurofibrillary tangles (NFTs) in the subject compared to a control.

Examples of tauopathies and conditions associated therewith that may be treated, ameliorated or prevented according to the described methods include, but are not limited to Alzheimer's disease, Argyrophilic grain disease (AGD), Chronic Traumatic Encephalopathy (CTE), Dementia pugilistica (chronic traumatic encephalopathy), frontotemporal dementia, frontotemporal lobar degeneration.gangliocytoma, Ganglioglioma, gangliocytoma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis,tangle-predominant dementia, lead encephalopathy, tuberous sclerosis, Hallervorden-Spatz disease, lipofuscinosis, corticobasal degeneration.

In some forms the tauopathy is a non-Alzheimer's tauopathy. Non-Alzheimer's tauopathies are sometimes grouped together as “Pick's complex.”

In some forms, the compositions are used to reduce, prevent or delay neurofibrillary tangles (NFTs), lactylated tau expression, tau phosphorylation, or pathologies associated with the aggregation of tau protein in the brain. The method can include administering to the subject an effective amount of a lactylated tau peptide to reduce, delay, or inhibit the expression of, lactylation of, or phosphorylation of tau in the subject compared to a control. The method can include treating subjects that have not yet been diagnosed with a specific disease or disorder.

i. Alzheimer's Disease

In some forms, the disclosed compositions and methods are used to treat Alzheimer's disease (AD). AD is one of the most widely studied proteinopathies. Genetic, pathological, biochemical, animal and cell modeling indicate that accumulation of misfolded, aggregated proteins in the brain triggers a complex series of events that result in neuronal degeneration. In AD aggregation and accumulation of the amyloid p (also referred to as p amyloid, beta amyloid, amyloid beta, Abeta and Aβ) protein and microtubule associated protein tau (MAPT) (also referred to herein as tau) have both been implicated as key pathogenic ‘triggers’. Aβ accumulates in senile plaques, cerebral vessels, and within neurons. Tau accumulates inside cells as neurofibrillary tangles (NFTs) and tau neuritis, and is often hyperphosporylated.

Tangle pathology is most pronounced in vulnerable brain regions in AD, including the cortex and hippocampus. Since mutations in tau produce known familial brain diseases such as frontal temporal dementia (FTD), it is believed that tau aggregation is linked to neuronal damage and cognitive dysfunction.

In genetic forms of AD, data support the “Ap aggregate/amyloid cascade” hypothesis, which postulates that Aβ aggregation and accumulation precedes, and therefore drives, tau accumulation and hyperphosporylation, supported by the observation that individuals with mutations in pathways that increase Aβ levels have familial AD, whilst evidence from drug trials, genetic studies, and experimental work in animal models indicates that an additional late-onset AD (LOAD) might exist in some cases.

A number of pharmacological agents are being developed to treat AD, for example, Aβ42 immunization, tarenflurbil (FLURIZAN™, Myriad Pharmaceuticals) which is believed to act by decreasing the production of Aβ42, and tramiprosate (ALZHEMED™, Neurochem Inc.) which was designed to bind to beta amyloid peptide and prevent it from reacting with glycosaminoglycans.

In some forms, the described methods and compositions reduce, prevent or delay neurofibrillary tangles (NFTs), lactylated tau expression, tau phosphorylation, or pathologies associated with AD in a subject in need thereof. In some forma, the described compositions and methods include administering to the subject an effective amount of a lactylated tau peptide having a defined lactyllysine to reduce, delay, or inhibit the expression of, lactylation of, or phosphorylation of tau in the subject compared to a control treatment in early Alzheimer's disease. In some forms, the methods include administering to a subject with AD an effective amount of a lactylated tau peptide having a defined lactyllysine to prevent or delay the onset of the disease based on the presence of disease predicting mutations, or plaques visible during brain imaging. Methods to monitor AD progression are known in the art, and include brain imaging of plaque burden.

### B. Combination Therapies

Combination therapies include administering the composition containing an effective amount of a lactylated tau peptide having a defined lactyllysine disclosed herein in combination with one or more second therapeutic agents. For example, the composition itself can include a combination of a lactylated tau peptide having a defined lactyllysine and one or more second therapeutic agents. In another form, a first composition including a lactylated tau peptide having a defined lactyllysine is co-administered with one or more additional compositions including one or more second therapeutic agents. Exemplary lactylated tau peptides include CQIINK(K-lactyl)LDLSN (SEQ ID NO:7) and CINK(K-lactyl)LDLSNVQSKC (SEQ ID NO:8; Tau K281la peptide B: 279-290).

The second therapeutic agent can be a conventional therapeutic agent for treating a tauopathy. The second agent can determined based on the disease to be treated. For example, if the disease is Alzheimer's disease, the compositions disclosed herein can be coadministered with a conventional Alzheimer's disease treatment such as Aβ42 immunization, tarenflurbil (FLURIZAN™, Myriad Pharmaceuticals) which is believed to act by decreasing the production of Aβ42, and tramiprosate (ALZHEMED™, Neurochem Inc.) which was designed to bind to beta amyloid peptide and prevent it from reacting with glycosaminoglycans.

### C. Administration

The compositions can be administered prophylactically, therapeutically, or combinations thereof. Therefore, the composition can be administered during a period before, during, or after onset of one or more symptoms of the disorders disclosed herein. In some forms, the composition is administered with one or more additional therapeutic agents as part of a co-therapy, one or more second treatments, or combinations thereof.

The disclosed compositions may be administered in a physiologically acceptable carrier to a host. Preferred methods of administration include systemic or direct administration to a cell. The compositions can be administered to a cell or patient, as is generally known in the art for protein therapies. One form provides a pharmaceutical composition including a lactylated tau peptide having a defined lactyllysine and a pharmaceutically acceptable carrier or excipient. In some forms, the lactylated tau peptide includes a defined lactyllysine at position 281 of SEQ ID NO:2. An exemplary peptide includes such as lactylated lysine at position K331 of Tau 441.

The composition typically includes an effective amount of the lactylated tau peptide having a defined lactyllysine to prevent, reduce, delay, or inhibit the level, formation, or production of amyloid proteins in subject; reduce the expression or accumulation of Abeta in a subject; reduce the ratio of Abeta42/Abeta40 in a subject; prevent, reduce, delay, or inhibit the expression of tau, or lactylation of tau, or phosphorylation of tau in a subject; limit the progression of, reduce, delay, or inhibit the level of neurofibrillary tangles (NFTs) in a subject; decrease neurodegeneration in the brain of a subject; increase the relative amount of non-lactylated tau in the brain of a subject; or a combination thereof.

The compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 17th edition, Osol, A. Ed. (198)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween®, Pluronics® or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, re-constitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

The composition can include one or more additional ingredients. As used herein, “additional ingredients” include: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other additional ingredients which may be included in the pharmaceutical compositions are known. Suitable additional ingredients are described in Remington's Pharmaceutical Sciences, 17th ed. Mack Publishing Co., Genaro, ed., Easton, Pa. (1985).

Dosages and desired concentrations of the polynucleotide-binding polypeptide disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

The composition can be administered intravenously in a wide dosing range from about 0.01 milligram per kilogram body weight (mg/kg) to about 10 mg/kg, alternatively about 0.01 milligram per kilogram body weight (mg/kg) to about 1.0 mg/kg, depending on patient's age and physical state, as well as dosing regimen and schedule.

The dose can be administered in separate administrations of 2, 3, 4, 5 or 6 doses. The dose can be administered every day, every two days, every three days, every four days, every five days, every six days, every seven days, once every two weeks, or once a month.

In some forms the composition is lyophilized and reconstituted in sterile water prior to use. In another form the composition is lyophilized in 20 mM histidine, 10 mg/mL trehalose, 30 mg/mL mannitol, pH 6.5 and reconstituted in sterile water prior to use. In yet another form, the composition is dissolved in 20 mM histidine, 150 mM NaCl pH 6.5 and kept frozen prior to use.

The invention can be further understood by the following numbered paragraphs:
1. A method of detecting or quantifying lactylation of tau protein, including detecting lactylated tau protein in a sample including tau protein.
2. The method of paragraph 1, wherein the detecting includes contacting the sample with one or more binding moieties that selectively bind lactylated tau protein.
3. The method of paragraph 2, wherein the tau protein is within a biological sample from a subject,
optionally wherein the biological sample includes cells or a cell lysate or a fraction thereof,
optionally wherein the cells or cell lysate are obtained as a biopsy from the subject.
4. The method of paragraph 3, wherein the biological sample includes a bodily fluid selected from the group consisting of Cerebral Spinal Fluid (CSF), blood, plasma, urine, saliva, nasal fluid, bone marrow, brain tissue, vomit and feces.
5. The method of paragraph 3, wherein the biological sample includes CSF.
6. The method of paragraph 3, further including determining that the biological sample includes lactylated tau protein if the level of detected binding is higher in the biological sample than in a control.
7. The method of paragraph 6, wherein the control includes a recombinant non-lactylated tau protein, or a biological sample including a tau protein from a healthy subject.
8. The method of paragraph 1, wherein binding is detected by a method selected from the group consisting of an immunoassay, immunohistochemistry, Western blotting, surface plasmon resonance, flow cytometry (FACS) analysis, mass spectrometry, chemical labeling and imaging, chromatographic methods, animal behavior based assays, biosensor analyses and biochip analyses.
9. The method of paragraph 8, wherein the immunoassay is selected from the group consisting of an enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, and immuno-chromatography assays.
10. The method of paragraph 1, wherein the one or more binding moieties do not bind to non-lactylated tau protein, optionally wherein the one or more binding moieties binds to lactylated lysine (Kla).
11. The method of paragraph 1, wherein the one or more binding moieties is selected from the group consisting of a small molecule, a protein, and a nucleic acid.
12. The method of paragraph 11, wherein the one or more binding moieties includes an antibody or an antigen binding fragment thereof.
13. The method of paragraph 12, wherein the antibody or antigen binding fragment thereof includes an antigen binding domain that immuno-specifically binds to one or more peptide selected form the group consisting of CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIIIH(K-lactyl)PGGGQVEC (SEQ ID NO:10); GNIHH(K-lactyl)PGGGQC (SEQ ID NO:11); CTHVPGGGN(K-lactyl)(K-lactyl)IETHK (SEQ ID NO:13); and CVPGGGN(K-lactyl)(K-lactyl)IETHK (SEQ ID NO:14).
14. A method of diagnosing a subject with a disease or disorder associated with lactylated tau protein, including detecting or quantifying lactylated tau protein within a sample from the subject, wherein the detecting includes contacting the sample with one or more binding moieties that selectively bind lactylated tau protein,
optionally further including diagnosing a subject with a disease or disorder if the level of detected binding is higher in the biological sample than in a control,
optionally wherein the control includes a recombinant non-lactylated tau protein, or a biological sample including a tau protein from a healthy subject or average of a pool from two or more subject.
15. The method of paragraph 14, wherein the disease or disorder associated with lactylated tau protein is selected from the group consisting of Alzheimer's disease, Argyrophilic grain disease (AGD), Chronic Traumatic Encephalopathy (CTE), Dementia pugilistica (chronic traumatic encephalopathy), frontotemporal dementia, frontotemporal lobar degeneration, gangliocytoma, Ganglioglioma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis,tangle-predominant dementia, lead encephalopathy, tuberous sclerosis, Hallervorden-Spatz disease, lipofuscinosis, and corticobasal degeneration.
16. The method of paragraph 14, wherein the disease or disorder is Alzheimer's disease.
17. The method of paragraph 14 further including treating the subject.
18. The method of paragraph 17, wherein the treatment includes a therapy effective for treating diseases and disorders characterized by the presence of lactylated tau protein.
19. The method of paragraph 17, wherein the treatment includes administering to the subject an effective amount of a lactylated tau protein or fragment thereof, to reduce or alleviate one or more symptoms of the disease or disorder.
20. The method of paragraph 16, further including administering to the subject an effective amount of a lactylated tau protein or fragment thereof, to reduce or alleviate one or more symptoms of Alzheimer's disease.
21. The method of paragraph 19, wherein the lactylated tau protein or fragment thereof is administered to the subject at dosage of between 0.01 mg/kg and about 1.0 mg/kg, inclusive.
22. The method of paragraph 19, wherein the lactylated tau protein or fragment thereof is administered to the subject every day, every two days, every three days, every four days, every five days, every six days, every seven days, once every two weeks, or once a month.
23. The method of paragraph 19, wherein the lactylated tau protein is selected from the group consisting of CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIIIH(K-lactyl)PGGGQVEC (SEQ ID NO:10); GNIHH(K-lactyl)PGGGQC (SEQ ID NO:11); CTHVPGGGN(K-lactyl)(K-lactyl)IETHK (SEQ ID NO:13); and CVPGGGN(K-lactyl)(K-lactyl)IETHK (SEQ ID NO:14).
24. The method of paragraph 19, wherein the lactylated tau protein is selected from the group consisting of CQIINK(K-lactyl)LDLSN (SEQ ID NO:7) and CINK(K-lactyl)LDLSNVQSKC (SEQ ID NO:8).
25. The method of paragraph 19, wherein the lactylated tau protein is CQIINK(K-lactyl)LDLSN (SEQ ID NO:7).
26. A composition including an isolated tau protein, or a fragment thereof, wherein the tau protein, or a fragment thereof includes lactylation of one or more lysine residues.
27. The composition of paragraph 26, wherein the tau protein includes an amino acid sequence of any one of SEQ ID NO:2,
wherein one or more lysine resides is lactylated,
wherein the one or more lactylated lysine residue is selected from the group consisting of K224, K225, K257, K267, K281, K311, K317, K331, and K321.
28. The composition of paragraph 26, wherein the tau protein includes an amino acid sequence of any one of SEQ ID NOs:3, or 32-40.
29. The composition of paragraph 26, wherein the tau protein includes an amino acid sequence of SEQ ID NO:36.
30. The composition of paragraph 26, wherein the tau protein or fragment thereof is in an amount effective for use as a positive control composition for an assay for diagnosing a subject with a disease or disorder associated with lactylated tau protein.
31. A kit including the composition of paragraph 30.
32. An antibody that selectively binds lactylated tau protein, or an antigen binding fragment thereof, wherein the antibody is prepared according to a method including:
(a) immunizing a subject with the recombinant tau protein of paragraph 26;
(b) isolating an antibody that immuno-specifically binds to the recombinant tau protein from the subject.
33. The antibody or antigen binding fragment thereof of paragraph 32, wherein the subject is immunized with one or more of SEQ ID NOs:3, 4-5, 7-8, 10-11, 13-14, or 32-40.
34. The antibody or antigen binding fragment thereof of paragraph 32, wherein the antibody is an intact antibody or functional antibody fragment or fusion protein.
35. The antibody or antigen binding fragment thereof of paragraph 34, wherein the functional fragment or fusion protein is selected from Fab fragments, F(ab′)2 fragments, Fab′ fragments, Fv fragments, recombinant IgG (rlgG) fragments, single chain antibody fragments optionally single chain variable fragments (scFv), and single domain antibodies optionally selected from sdAb, sdFv, and nanobody fragments.
36. The antibody or antigen binding fragment thereof of paragraph 32, wherein the antibody is selected from the group consisting of intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv.
37. The antibody or antigen binding fragment thereof of paragraph 32, wherein the antibody is an IgM, IgE, IgA, IgD, or IgG optionally an IgG₁, IgG₂, IgG₃, or IgG4.

### EXAMPLES

### Example 1: Lactylation of Tau in Human Alzheimer's Disease Brains

Zhang X, Liu Y, Rekowski M J, Wang N. Lactylation of tau in human Alzheimer's disease brains. Alzheimers Dement. 2025 February; 21(2):e14481. doi: 10.1002/alz.14481. Epub 2024 Dec. 30. PMID: 39740133; PMCID: PMC11851134; is specifically incorporated by reference herein in its entirety including all supplemental materials.

### Materials and Methods

### Patient Samples

Autopsy brain tissue samples from the frontal cortex were provided by the Neuropathology Core of the University of Kansas Alzheimer's Disease Research Center (KU ADRC). Samples were homogenized on dry ice and subsequently aliquoted for analysis. Detailed information regarding the human samples utilized in this study, including the AD and control cases, is provided in Table 1.

TABLE 1
Human sample information

| ID | Sex | Age | Diagnosis | Tissue |
|---|---|---|---|---|
| ADC003 | M | 82 | ND | FCx, Superior B1 |
| ADC005 | M | 89 | ND | FCx, Superior B1 |
| ADC007 | M | 86 | AD/CAA/TDP43+ | FCx, Superior B1 |
| ADC011 | M | 87 | AD/LB | FCx, Superior B1 |
| ADC013 | M | 78 | AD/CAA | FCx, Superior B1 |
| ADC014 | M | 91 | ND | FCx, Superior B1 |
| ADC040 | M | 74 | CN/Hem/loAD | SFG |
| ADC044 | M | 67 | CN/loAD | SFG |
| ADC054 | M | 95 | CN | SFG |
| ADC056 | M | 74 | AD | SFG |
| ADC059 | M | 68 | AD | SFG |
| ADC086 | M | 92 | AD/ARTAG | SFG |

Note:
FCx = frontal cortex;
SFG = superior frontal gyrus;
ND = non-demented;
CAA = cerebral amyloid angiopathy;
LB = Lewy body;
CN = control non-demented;
load = low AD;
ARTAG = aging-related tau astrogliopathy;
Hem = hemorrhage

### Antibodies

Anti-L-lactyllysine (PTM-1401RM) antibody was purchased from PTBIO. Anti-AT8 (MN1020) antibody was purchased from Thermo Fisher Scientific. Anti-tau (210-241) T (Tau-5, MAB361), antitau^{368N} (ABN-1703), anti-Flag tag (F01804), and ant-HA tag (H6908) antibodies were purchased from Sigma. Anti-GFP (ab290) antibody was purchased from Abcam. Anti-Actin (66009-1-Ig), Anti-His tag (66005-1-Ig), and Anti-pan-acetylation (66289-1-Ig) antibodies were purchased from Proteintech Group. Information relating to these antibodies, including their dilutions used in the experiments, is provided in Table 2.

TABLE 2
List of Antibodies

| Antibodies | | | Dilution |
|---|---|---|---|
| Pan-lactyllysine | PTM BIO | PTM-1401RM | 1:1,000 |
| AT8 | Thermo Fisher | MN1020 | 1:2,000 |
| | Scientific | | |
| Tau5 | Sigma | MAB361 | 1:1,000 |
| Tau368N | Sigma | ABN-1703 | 1:5,000 |
| Flag tag | Sigma | F1804 | 1:5,000 |
| HA tag | Sigma | H6908 | 1:5,000 |
| GFP tag | Abcam | ab290 | 1:5,000 |
| Actin | Proteintech | 66009-1-Ig | 1:10,000 |
| His tag | Proteintech | 66005-1-Ig | 1:5,000 |
| Pan-acetylation | Proteintech | 66289-1-Ig | 1:1,000 |
| Goat anti Rabbit | Bio rad | 1706515 | 1:10,000 |
| Goat anti Mouse | Bio rad | 1706516 | 1:10,000 |
| donkey anti-mouse | Life Technologies | Cat# A21202; | 1:500 |
| IgG Alexa Flour 488 | | RRID: AB_141607 | |
| donkey anti-rabbit IgG | Life Technologies | Cat# A10042; | 1:500 |
| Alexa Flour 568 | | RRID: AB_2534017 | |
| donkey anti-goat IgG | Life Technologies | Cat# A21447; | 1:500 |
| Alexa Flour 647 | | RRID: AB_141844 | |

### Plasmids

The following plasmids were used in this study: TauWT plasmid (Addgene plasmid #14748), pRK5-EGFP-Tau AP (Addgene plasmid #46905) [10], pSG5-HA-p300 (Addgene plasmid #89094) [11], HA-Ubiquitin (Addgene plasmid #18712) [12], pAdDeltaF6(Addgene plasmid #112867), pAAV2/8 (Addgene plasmid #112864), and AAV.CBA.eGFP.2A.wtTau (Addgene plasmid #140424) [13]. Tau^{3KR} plasmid was generated at Gene Universal.

### Cell Lines

HEK 293T cell lines were procured from the American Type Culture Collection (ATCC). Cells were maintained in a humidified incubator set at 37° C. with 5% CO₂. Cells underwent validation via short tandem repeat (STR) DNA profiling and were confirmed to be free of mycoplasma contamination through PCR testing. Culture media (DMEM) were supplemented with 10% fetal bovine serum (FBS) and 1% penicillin-streptomycin to support optimal growth conditions. Mouse primary neuronal cultures were prepared from cortices of postnatal day 1 pups. Cells were purified and plated at 0.1 mg/ml poly-D-lysine coated dish in neurobasal medium supplemented with B27. Experimental treatments were conducted at 7-13 days in vitro (DIV) in neurobasal medium supplemented with N2.

Recombinant AAV-TauWT was generated by co-transfection of the AAV plasmids with helper plasmids, including the AAV2/8 vector, pAdDeltaF6 vector and AAV.CBA.eGFP.2A.wtTau vector, into 293T cells. AAV were collected at 72 h and infected primary neurons.

For Western blot assays, cells were treated with various compounds under the following conditions: L-lactate at 2 mM, DCA at 20 mM, 2-DG at 10 mM, rotenone at 10 mM, CHX at 100 mg/mL, MG132 at 20 μM. All treatments were applied for a duration of 24 hours.

Transient transfection of DNA plasmids into HEK 293T cells was performed using Lipofectamine 3000 (Thermo Fisher Scientific). Six hours post-transfection, the media was replaced with fresh complete media.

For lentiviral transduction, HEK 293T were infected with control or LDHA shRNA lentivirus. Harvested lentiviruses were added to the cells for further experiments with 8 μg/ml polybrene to enhance infection efficiency.

### Western Blot Analysis

Total protein was extracted using RIPA buffer supplemented with 1 mM PMSF (Sigma) and a protease inhibitor cocktail (Sigma P8340). Following centrifugation at 12,000×g for 15 minutes at 4° C., the lysates were collected. The pellet was washed with ice-cold RIPA buffer, followed by lysis in 10% SDS buffer (10% SDS, 250 mM Tris, pH 6.8). The lysates were sonicated in a water bath sonicator until no particulate material remained. Protein concentrations were determined using the bicinchoninic acid (BCA) method for both RIPA-soluble and RIPA-insoluble fractions. Equal amounts of protein from each sample were mixed with LDS sample buffer (Invitrogen) and reducing agent (Invitrogen), then denatured at 70° C. for 10 minutes. The samples were resolved on 4-12% Bis-Tris gels (Thermo Fisher) and transferred to PVDF membranes. Membranes were incubated overnight at 4° C. with primary antibody at a 1:1,000 dilution, followed by washing and incubation with a secondary antibody. Protein detection was carried out using Clarity ECL Western Blotting Substrate (Bio-Rad).

### Immunohistochemistry and Immunofluorescence

For immunohistochemistry, formalin-fixed, paraffin-embedded (FFPE) human tissue sections were dewaxed and rehydrated through a graded ethanol series, followed by microwave-assisted antigen retrieval in 0.01 M citrate buffer (pH 6.0) and methanol/H₂O₂ treatment. Non-specific binding was blocked with 5% goat serum before incubation with primary antibody and HRP-conjugated secondary antibodies. Staining was visualized using the NovaRed substrate kit (Vector, SK-4800).

### Immunoprecipitation and Pull-Down

For transient transfection and co-immunoprecipitation assays, HEK 293T cells were co-transfected with plasmids encoding GFP-tagged wild-type tau or HA-tagged p300. The transfected cells were lysed on ice for 25 minutes using NETN buffer (20 mM Tris-HCl, pH 8.0, 100 mM NaCl, 1 mM EDTA, and 0.5% Nonidet P-40) supplemented with 1× protease inhibitors. Following centrifugation at 12,000 rpm for 10 minutes to remove cell debris, the soluble fractions were collected and incubated with HA beads for 2 hours at 4° C. The beads were washed three times with NETN buffer, then boiled in LDS loading buffer for 10 minutes and subjected to SDS-PAGE. Membranes were blocked with 5% milk in TBST buffer and probed with indicated antibodies.

For in vitro pull-down assay, His-tagged purified tau protein and Flag-tagged p300 were added in the NETN buffer. The mixture was incubated for 30 minutes at 4° C. Subsequently, Flag beads were added to capture the protein complexes and incubated for an additional 30 minutes at 4° C. The beads were washed three times with the NETN buffer and then denatured at 70° C. for 10 minutes.

### Flow Cytometry

Single-cell suspensions were prepared from cultured cells after incubation in serum-free DMEM media containing 100 M SCOTfluor lactic acid probe 510, for 30 minutes at 37° C. Then, cells were washed in PBS and stained with DAPI to exclude dead cells. Flow cytometry analysis was performed using a BD FACS Aria III.

### In Vitro Tau Lactylation and Acetylation

The recombinant His-tagged tau proteins (Abcam) was incubated with Flag-tagged p300 proteins (R&D Systems) in reaction buffer (50 mM HEPES, pH7.8, 30 mM KCl, 0.25 mM EDTA, 5.0 mM MgCl2, 5.0 mM sodium butyrate, 2.5 mM DTT) with 20 M acetyl-CoA (Sigma) or lactyl-CoA (MCE). Reactions were incubated at 30° C. for 30 min. Next, LDS loading buffer was added to the reaction and denatured at 70° C. for 10 min. Samples were separated by SDS-PAGE and immunoblotted with indicated antibodies.

### In Vitro Tau Ubiquitination

The recombinant tau proteins (Abcam) were ubiquitinated using the CHIP ubiquitin ligase kit (Misfolder Protein Ubiquitination Kit, BPBio Cat. #J5110) in the presence of E1, E2 (UBE2D3), and E3 CHIP enzymes. Briefly, 6 μL of recombinant tau protein (1 μg/L), adding 6 μL each of 10×E1 enzyme, 10×E2 (UBE2D3) enzyme, and 10×CHIP to initiate ubiquitination. The mixture was vortexed, briefly spun down, and incubated at 37° C. for 6 hours. To terminate the reaction, the tubes were placed on ice for 10 minutes, followed by heat inactivation at 70° C. for 15 minutes.

### In Vitro Tau Cleavage

The recombinant tau proteins (Abcam) were incubated with rAEP proteins (Sino Biological) in reaction buffer. Reactions were incubated at 37° C. for 30 min. Next, LDS loading buffer was added to the reaction and denatured at 70° C. for 10 min. Samples were separated by SDS-PAGE and immunoblotted with indicated antibodies.

### Docking Modeling Studies

The structure of lactyl-CoA (CHEBI ID: 15529) was obtained in SMILES format from the ChEBI database. This format was then converted to PDB format using Chem3D for subsequent molecular docking studies. Receptor proteins were downloaded in PDB format p300 (PDB Accession No. 6GYR). Protein preparation was performed using AutoDock Tool 4.2.1, and molecular docking was carried out with AutoDock Vina, which automatically calculates grid maps. Following successful docking, ligand binding positions and bond distances were analyzed and confirmed using PyMOL and LigPlot. Both PyMOL and LigPlot facilitated detailed visualization of ligand-protein interactions, including polar bonds, bond distances, and hydrophobic interactions.

### Data Analysis of Mass Spectrometry

Data were downloaded from ProteomeXchange (PXD020517) and searched with Proteome Discoverer 3.0 [14] using the Sequest algorithm against the human Tau protein sequence and a database of common contaminants. Precursor mass tolerance was set to 10 ppm and fragment mass tolerance set to 0.02 Da. The data were searched allowing a maximum of 4 modifications per peptide including variable modifications: oxidation (Met, +15.9949), acetylation (Lys, +42.0105), ubiquitination (Lys, +114.0429), lactylation (Lys, +72.0211), phosphorylation (Ser, Thr, Tyr, +79.9663) and static modification on cysteine (propionamide, +71.0371).

### Mass Spectrometry Analysis of In Vitro Lactylated Tau

After incubation of Tau with P300, proteins were alkylated with iodoacetamide (10 mM final) in the dark for 10 min at room temperature. Ice cold acetone was added (1:4) to the sample and incubated overnight at −20° C. to precipitate the proteins. The proteins were pelleted by centrifuging at 14, 000×g for 30 min at 4° C. The protein pellet was washed 2× with ice cold acetone and the pellet dried on the benchtop for 10 min. The pellet was resuspended in 50 mM TEAB, 2 mM CaCl₂ buffer prior to digestion with trypsin overnight at 37° C. with shaking at 500 RPM (Thermomixer, Eppendorf). The digestion was quenched with the addition of formic acid to 1%. Peptides were quantitated by Nanodrop spectrophotometry at 205 nm before LC-MS/MS analysis.

The peptide sample was injected using the Vanquish Neo (Thermo) nano-UPLC onto a C18 trap column (PEPMAP™ Neo Trap, 0.3 mm×5 mm, 5 μm particle size) using pressure loading. Peptides were eluted onto the separation column (PEPMAP™ Neo, 75 μm×150 mm, 2 μm C18 particle size, Thermo) prior to elution directly to the mass spectrometer. Briefly, peptides were loaded and washed for 5 minutes at a flow rate of 0.350 μL/min at 2% B (mobile phase A: 0.1% formic acid in water, mobile phase B: 80% ACN, 0.1% formic acid in water). Peptides were eluted over 60 minutes from 2-30% mobile phase B before ramping to 45% B in 5 min. The column was washed for 10 min at 100% B before re-equilibrating at 2% B for the next injection. The nano-LC was directly interfaced with the Orbitrap Ascend Tribrid mass spectrometer (Thermo) using a silica emitter (20 μm i.d., 10 cm, CoAnn Technologies) equipped with a high field asymmetric ion mobility spectrometry (FAIMS) source (Thermo). The data were collected by data dependent acquisition with the intact peptide detected in the Orbitrap at 120,000 resolving power from 375-1500 m z. Peptides with charge +2-7 were selected for fragmentation by higher energy collision dissociation (HCD) at 28% NCE and were detected in the Orbitrap at 30,000 resolving power. Dynamic exclusion was set to 60s after one instance. The mass list was shared between the FAIMS compensation voltages. FAIMS voltages were set at −45 (1.4 s), −60 (1 s), −75 (0.6 s) CV for a total duty cycle time of 3s. Source ionization was set at +1700 V with the ion transfer tube temperate set at 305° C. Raw files were searched against the human tau protein sequence and a common contaminants database using SEQUEST in Proteome Discoverer 3.0 [14]. Abundances, abundance ratios, and p-values were exported to Microsoft Excel for further analysis. Spectra were exported directly from Proteome Discoverer 3.0.

### Quantification and Statistical Analysis

All experiments were replicated at least three times independently. Quantitative data from the experimental replicates were pooled and are presented as mean±SE as indicated in the figure legend. Normality testing was conducted using Kolmogorov-Smirnov tests. Compiled data were analyzed by Student's t test and two-way ANOVA test.

### Results

### Elevated Lactylation of Tau in the Human AD Brain

To identify AD-associated changes in the expression of lactate signature genes (genes encoding regulators of lactate metabolism), a single nuclei RNA-seq (snRNA-seq) dataset from control and AD human brain samples was analyzed [15]. Pooled cell transcriptomes from ND and AD samples were used to define seven clusters. Importantly, cells from inhibitory (INH) neurons, excitatory (EX) neurons, oligodendrocytes (ODC), oligodendrocyte progenitor cell (OPC), and microglia (MG) clusters exhibit a significant and coordinated upregulation of lactate signature gene expression.

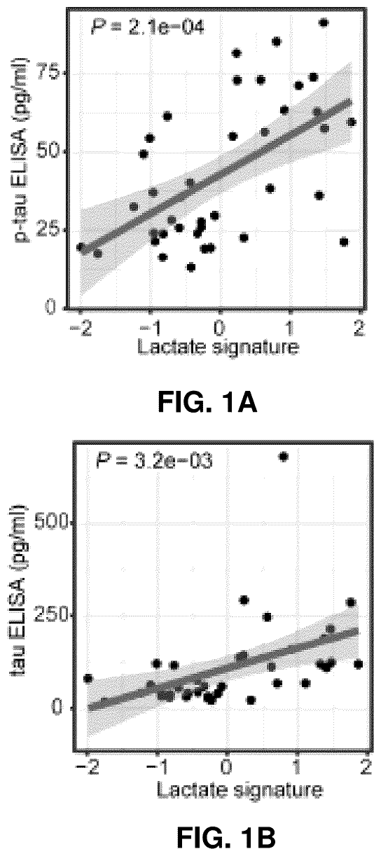

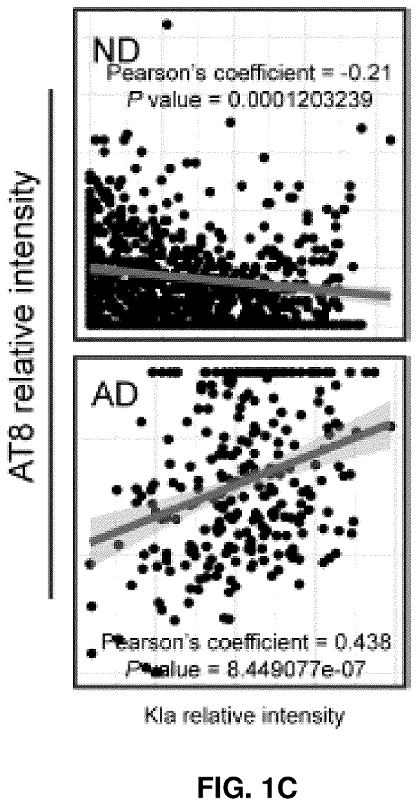

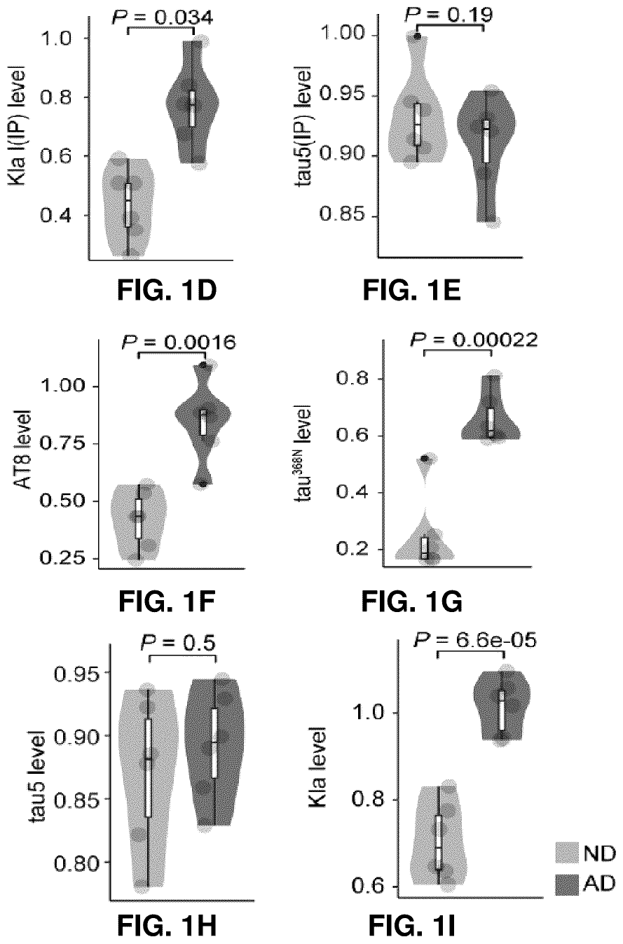

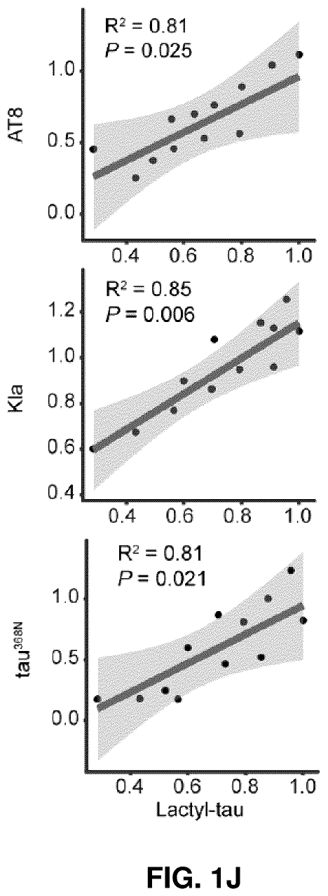

Next, a proteomics dataset was investigated for lactate signature expression in cerebrospinal fluid (CSF) samples from 19 control and 20 AD patients (patient information is provided in Table 3) [16]. The data show that lactate signature expression is upregulated in AD CSF. Moreover, lactate metabolism-related functions show a profound upregulation as well. Interestingly, the data show that the expression levels of tau and phosphorylated tau (p-tau) detected by ELISA significantly correlate with lactate signature expression (FIGS. 1A-1B).

TABLE 3
Patient information for CSF samples

| Sample ID | Diagnosis | Gender | Age | Race | APOE | Abeta | Tau | pTau |
|---|---|---|---|---|---|---|---|---|
| AD_CSF40_b1.128N | AD | Female | 59 | Caucasian | E4/4 | 152.75 | 109.17 | 35.98716 |
| | | | | or White | | | | |
| AD_CSF40_b1.129N | AD | Male | 75 | Caucasian | E3/4 | 163.87 | 118.90 | 59.32894 |
| | | | | or White | | | | |
| AD_CSF40_b1.130N | AD | Female | 52 | Caucasian | E3/3 | 267.95 | 187.86 | 62.53397 |
| | | | | or White | | | | |
| AD_CSF40_b1.131N | AD | Male | 63 | Black or | E4/4 | 214.65 | 53.84 | 36.85576 |
| | | | | African | | | | |
| | | | | American | | | | |
| AD_CSF40_b2.128C | AD | Male | 65 | Black or | E4/4 | 133.81 | 67.79 | 71.11238 |
| | | | | African | | | | |
| | | | | American | | | | |
| AD_CSF40_b2.129N | AD | Male | 90+ | Caucasian | E3/4 | 219.06 | 142.45 | 81.41084 |
| | | | | or White | | | | |
| AD_CSF40_b2.129C | AD | Female | 66 | Caucasian | E4/4 | 216.61 | 136.77 | 54.88685 |
| | | | | or White | | | | |
| AD_CSF40_b2.131N | AD | Male | 75 | Caucasian | NA | 343.90 | 679.25 | 85 |
| | | | | or White | | | | |
| AD_CSF40_b3.128C | AD | Female | 50 | Caucasian | E3/4 | 382.68 | 291.49 | 72.74992 |
| | | | | or White | | | | |
| AD_CSF40_b3.130N | AD | Female | 68 | Caucasian | E3/3 | 305.50 | 246.49 | 72.84288 |
| | | | | or White | | | | |
| AD_CSF40_b3.130C | AD | Male | 78 | Caucasian | E3/4 | 91.37 | 119.35 | 73.69732 |
| | | | | or White | | | | |
| AD_CSF40_b3.131C | AD | Female | 50 | Black or | E3/3 | 220.67 | 115.06 | 61.22256 |
| | | | | African | | | | |
| | | | | American | | | | |
| AD_CSF40_b4.128C | AD | Male | 76 | Caucasian | E4/4 | 189.53 | 119.79 | 54.24436 |
| | | | | or White | | | | |
| AD_CSF40_b4.129C | AD | Male | 75 | Caucasian | E4/4 | 155.18 | 123.35 | 57.31011 |
| | | | | or White | | | | |
| AD_CSF40_b4.130N | AD | Male | 56 | Black or | E4/4 | 197.44 | 67.81 | 38.21997 |
| | | | | African | | | | |
| | | | | American | | | | |
| AD_CSF40_b4.131N | AD | Female | 52 | Caucasian | E3/3 | 321.47 | 213.71 | 91.06036 |
| | | | | or White | | | | |
| AD_CSF40_b5.128N | AD | Male | 74 | Caucasian | E4/4 | 260.70 | 285.26 | 21.21841 |
| | | | | or White | | | | |
| AD_CSF40_b5.129N | AD | Female | 61 | American | E3/3 | 206.43 | 157.77 | 63.13478 |
| | | | | Indian or | | | | |
| | | | | Alaska | | | | |
| | | | | Native | | | | |
| AD_CSF40_b5.131N | AD | Male | 75 | Black or | E4/4 | 223.44 | 59.03 | 29.58653 |
| | | | | African | | | | |
| | | | | American | | | | |
| AD_CSF40_b5.131C | AD | Male | 51 | Black or | E3/3 | 321.52 | 111.43 | 56.22973 |
| | | | | African | | | | |
| | | | | American | | | | |
| AD_CSF40_b1.128C | Normal | Male | 61 | Caucasian | E3/4 | 663.71 | 61.89 | 49.25577 |
| | | | | or White | | | | |
| AD_CSF40_b1.129C | Normal | Female | 55 | Black or | E2/4 | 520.72 | 29.38 | 16.41763 |
| | | | | African | | | | |
| | | | | American | | | | |
| AD_CSF40_b1.130C | Normal | Female | 68 | Caucasian | E3/3 | 553.58 | 36.76 | 23.76985 |
| | | | | or White | | | | |
| AD_CSF40_b2.128N | Normal | Male | 82 | Caucasian | E3/3 | 464.33 | 16.98 | 17.5186 |
| | | | | or White | | | | |
| AD_CSF40_b2.130N | Normal | Male | 49 | Caucasian | E3/3 | 616.85 | 40.33 | 35.41487 |
| | | | | or White | | | | |
| AD_CSF40_b2.130C | Normal | Female | 66 | Black or | E3/2 | 445.04 | 26.67 | 27.67817 |
| | | | | African | | | | |
| | | | | American | | | | |
| AD_CSF40_b2.131C | Normal | Female | 70 | Black or | E3/3 | 467.80 | 22.60 | 19.15676 |
| | | | | African | | | | |
| | | | | American | | | | |
| AD_CSF40_b3.128N | Normal | Male | 69 | Black or | E3/3 | 467.35 | 22.96 | 22.56 |
| | | | | African | | | | |
| | | | | American | | | | |
| AD_CSF40_b3.129N | Normal | Male | 80 | Caucasian | E3/4 | 553.53 | 44.17 | 13.1215 |
| | | | | or White | | | | |
| AD_CSF40_b3.129C | Normal | Male | 81 | Caucasian | E3/3 | 608.82 | 76.20 | 40.07387 |
| | | | | or White | | | | |
| AD_CSF40_b3.131N | Normal | Male | 67 | Caucasian | E3/3 | 745.79 | 54.89 | 28.15266 |
| | | | | or White | | | | |
| AD_CSF40_b4.128N | Normal | Female | 62 | Caucasian | E3/4 | 690.64 | 31.71 | 25.74805 |
| | | | | or White | | | | |
| AD_CSF40_b4.129N | Normal | Male | 76 | Caucasian | E3/3 | 492.44 | 40.22 | 19.35767 |
| | | | | or White | | | | |
| AD_CSF40_b4.130C | Normal | Female | 72 | Black or | E3/3 | 402.43 | 40.66 | 32.3148 |
| | | | | African | | | | |
| | | | | American | | | | |
| AD_CSF40_b4.131C | Normal | Female | 60 | Caucasian | E3/3 | 528.27 | 32.92 | 21.42446 |
| | | | | or White | | | | |
| AD_CSF40_b5.128C | Normal | Female | 80 | Black or | E2/3 | 596.07 | 31.75 | 25.96308 |
| | | | | African | | | | |
| | | | | American | | | | |
| AD_CSF40_b5.129C | Normal | Male | 79 | Caucasian | E2/3 | 650.64 | 79.92 | 19.48168 |
| | | | | or White | | | | |
| AD_CSF40_b5.130N | Normal | Male | 64 | Caucasian | E3/3 | 495.68 | 45.22 | 24.02655 |
| | | | | or White | | | | |
| AD_CSF40_b5.130C | Normal | Male | 72 | Black or | E3/3 | 598.98 | 59.75 | 23.87194 |
| | | | | African | | | | |
| | | | | American | | | | |

Lactate has been shown to induce a PTM in lysine residues on histones and proteins, known as lysine lactylation (Kla) [17]. Immunohistochemistry (IHC) using an antibody against lactylated lysine (Kla) in histological sections from human postmortem patients (Table 1) showed a few cells positive for Kla in ND samples; in contrast, a significant increase in the number of cells positive for Kla was observed in AD samples, especially in neurons.

To examine whether lysine lactylation is associated with tau phosphorylation, sections from human postmortem control and AD brains were stained using the Kla antibody with an antibody against phosphorylated tau at Ser202 and Thr205 (AT8). These data show that proteins had undergone Kla as PTM in cells with phosphorylated tau (FIG. 1C).

Since tau often undergoes PTMs, which play important roles in regulating its functions [9], it was tested whether lysine lactylation could occur on tau. Thus, proteins were extracted from postmortem human ND and AD brain samples (Table 1) and isolated tau by immunoprecipitation by using an antibody against total tau (tau5). These data show that tau isolated from the AD samples exhibited increased levels of lysine lactylation, as detected by the Kla antibody. It was observed that tau isolated from the AD samples showed increased tau cleavage (detected by tau^{368N} antibody) and tau phosphorylation (detected by AT8 antibody) (FIGS. 1D-1I). Moreover, the levels of tau phosphorylation, tau cleavage, and total lactylation in each sample significantly correlate with the levels of tau lactylation in these samples (FIG. 1J).

### Tau Lactylation at K331 is Elevated in Human AD Brain

To identify the lysine lactylation sites in tau associated with AD, lysine lactylation as a PTM of tau was analyzed using a published, publicly available quantitative proteomics (MS) dataset by mass spectrometry that analyzed tau PTMs in postmortem control and AD human brain samples from parietal cortex (Brodmann area, BA39—angular gyrus) [18]. Given that tau PTMs did not exhibit any difference in the soluble fraction [18], this analysis was focused on tau lactylation in the insoluble fraction. Due to the corruption of some raw data, which were unusable for analysis, data from 47 out of 49 AD samples and 38 out of 42 control samples was analyzed.

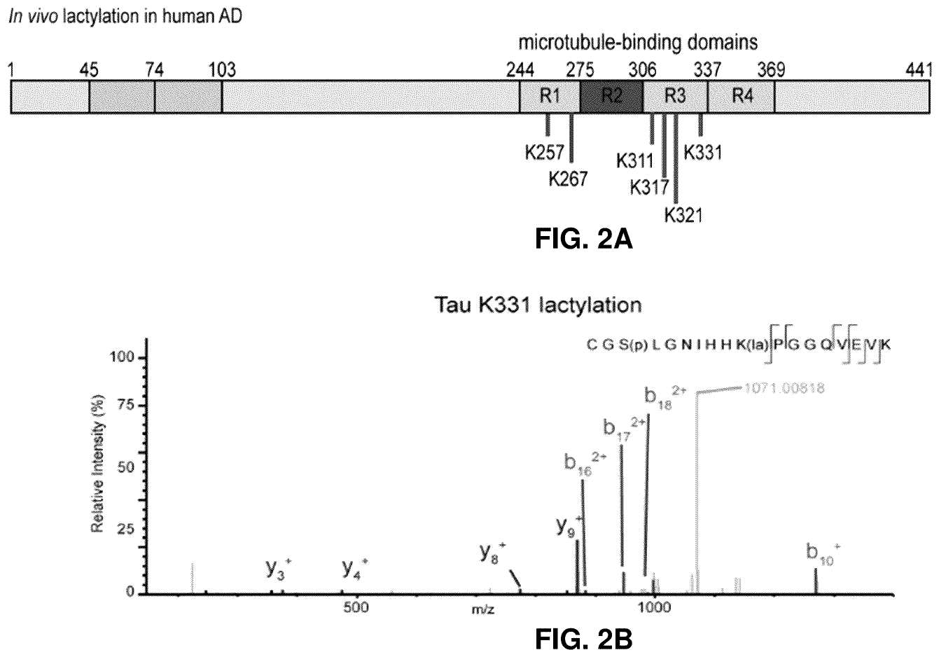

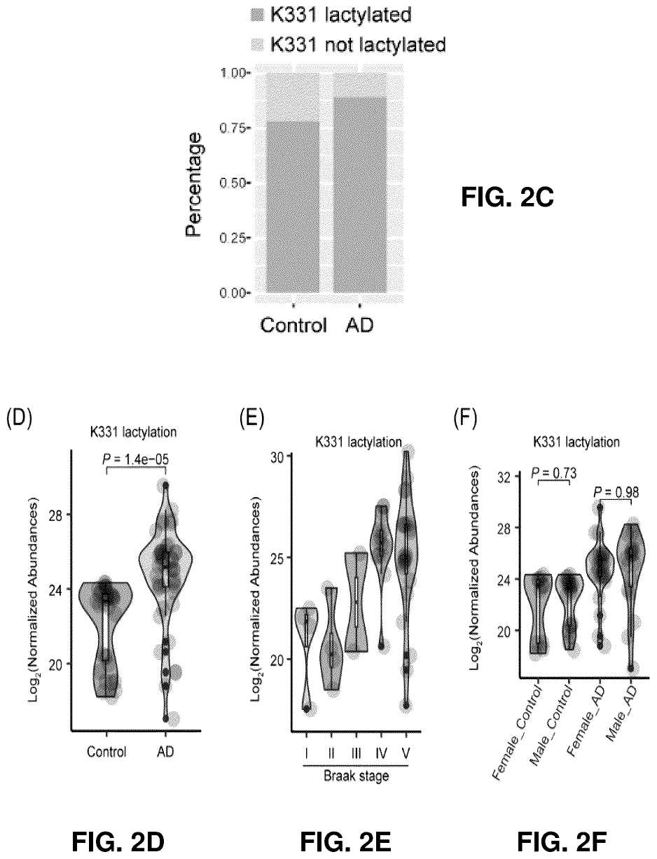

This analysis identified six distinct lactylation sites (lysine residues) on tau, all within the microtubule-binding domain (FIG. 2A). Peptides were identified with combinatorial modifications including lactylation at lysine residue at position 311 (K311) (#peptide spectrum match or PSM=3) and K317 or K321 (#PSM=2) was only detected in the AD samples but not in the control samples. Lactylation at K257 (#PSM=26) was detected in 41 out of 47 (83%) AD samples and 25 out of 38 (66%) control samples. Lactylation at K321 (#PSM=3) was detected in 7 out of 47 (15%) AD samples and 3 out of 38 (8%) control samples.

Notably, lactylation at K331 (#PSM=30) was detected in 44 out of 47 (94%) AD samples and 31 out of 38 (82%) control samples with a significantly increased abundance ratio (log₂=2.3; P-value: 0.00104) (FIG. 2B-2D). To assess heterogeneity and disease progression reflected by tau lactylation profiles, progressively higher levels of K331 lactylation was observed corresponding to Braak stages (control samples: Braak stages I, II and III; AD samples: Braak stages IV and V) (FIG. 2E). No significant changes were observed between male and female groups (FIG. 2F).

Taken together, these data from qualitative and quantitative proteomics by MS establish that tau is lactylated in the human brain and that tau lactylation is elevated in AD.

### Lactate Induces Tau Lactylation

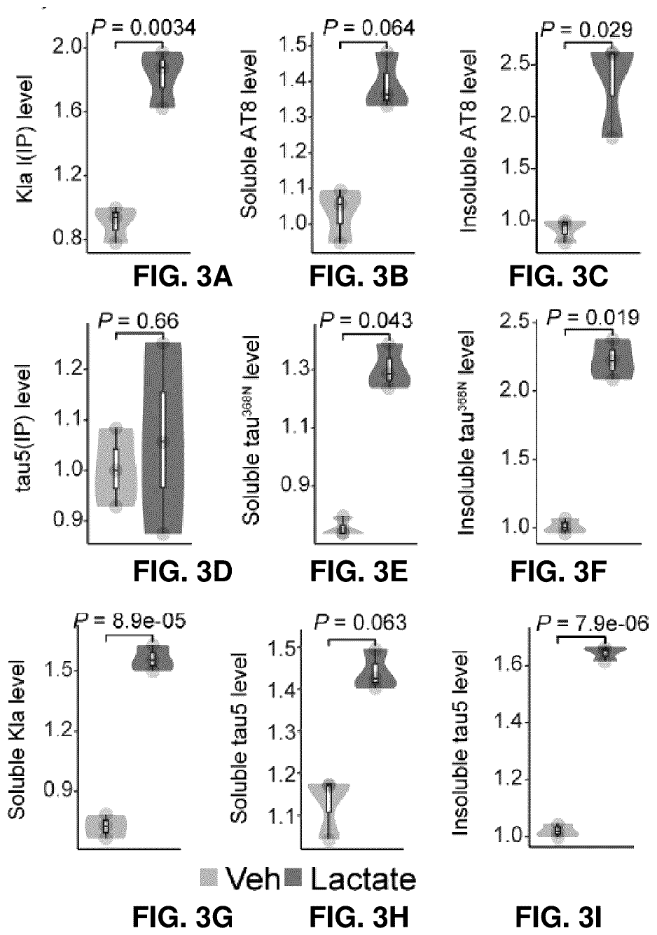

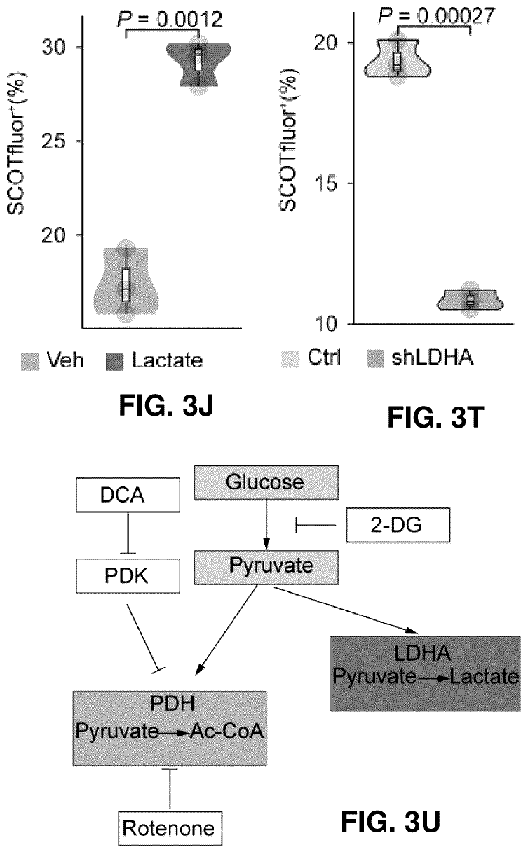

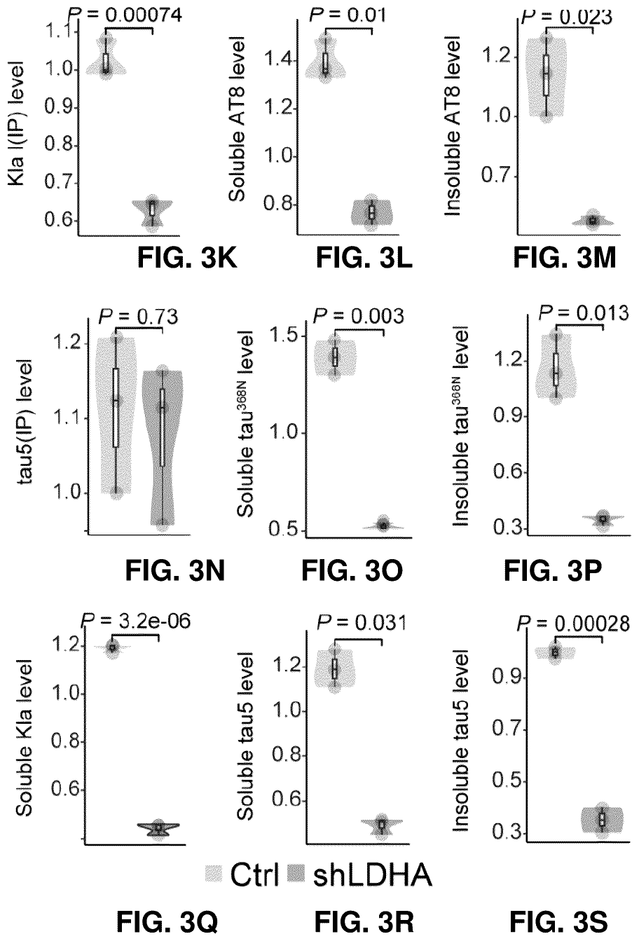

To examine whether tau lactylation is induced by lactate, human embryonic kidney (HEK) 293T cells were next transfected with a plasmid expressing wild-type tau-383. HEK 293 cells have been frequently used as a tool cell line for the studies of tau [19]. Tau-383 is a splicing variant human tau with 383 amino acids, which include the 4 microtubule binding regions (0N4R) but lack the N-terminal repeats [20]. After treating the transfected cells with lactate (2 mM; 24 hours), a significantly increased level of lactylation was detected in the immunoprecipitated tau by using the Kla antibody (FIGS. 3A-3I). Interestingly, lactate also induced tau phosphorylation, which was detected by using the AT8 antibody, and cleavage, which was detected by the tau^{368N} antibody, which occurred most profoundly in the insoluble fraction (FIGS. 3A-3I). Intracellular lactate levels were quantified using the SCOTfluor lactate probe, which confirmed that the lactate-treated cells exhibited increased intracellular lactate accumulation (FIG. 3J). To confirm these results in neurons, wild-type tau was expressed in primary mouse neuronal cell culture. After lactate treatment, an increased level of lactylation was detected in the immunoprecipitated tau by using the Kla antibody as well as increased tau phosphorylation in the insoluble fraction. Thus, these data suggest that lactate is an inducer of tau lactylation, phosphorylation, and cleavage.

Lactate dehydrogenase A (LDHA) produces a subunit of the lactate dehydrogenase enzyme necessary for lactate production in cells [21]. Since these data detected an endogenous level of tau lactylation in cells (FIGS. 3A-3I), it was determined whether this requires LDHA by knocking down LDHA using shRNA. After that, cells were transfected with wild-type tau-383. These data show a significantly decreased level of lactylation in the immunoprecipitated tau by using the Kla antibody (FIG. 3K-3S). These data suggest that inhibiting lactate production reduces tau lactylation. Interestingly, knockdown of LDHA also reduced tau phosphorylation and tau cleavage, with these effects being most pronounced in the insoluble fraction (FIG. 3K-3S). Intracellular lactate levels were quantified after LDHA knockdown, which confirmed decreased intracellular lactate accumulation (FIG. 3T). This observation suggests that LDHA-mediated lactate production induces tau lactylation.

Glucose is a precursor of lactate when glycolysis is not followed by oxidative phosphorylation, leading to the conversion of pyruvate into lactate under anaerobic conditions [22](FIG. 3U). Given the role of intracellular lactate in driving tau lactylation, HEK 293T cells were treated with the non-metabolizable glucose analogue 2-deoxy-D-glucose (2-DG) (FIG. 3U), which led to a reduction in tau lactylation. Concurrently, 2-DG treatment also downregulated tau phosphorylation and cleavage. Moreover, since lactate production is regulated by the balance between glycolysis and mitochondrial metabolism [23], it was investigated whether modulating the activity of key enzymes in these pathways could influence tau lactylation through changes in lactate levels. Sodium dichloroacetate (DCA), an inhibitor of pyruvate dehydrogenase kinase, decreased intracellular lactate [24] (FIG. 3U), leading to reductions in both tau lactylation and tau phosphorylation. Conversely, treatment with rotenone, a mitochondrial complex I inhibitor that shifts cellular metabolism toward glycolysis [25], upregulated tau lactylation. These findings collectively indicate that endogenous lactate production is an important determinant of tau lactylation levels.

### In Vitro Lactylation of Tau

p300 is an acetyl-transferase that has been demonstrated to mediate the transfer of lactyl-CoA to lysine residues in proteins, facilitating lactylation [26]. An in silico molecular docking was conducted [27], which revealed a strong binding affinity of lactyl-CoA to p300 (docking energy −20.417 Kcal/Mol).

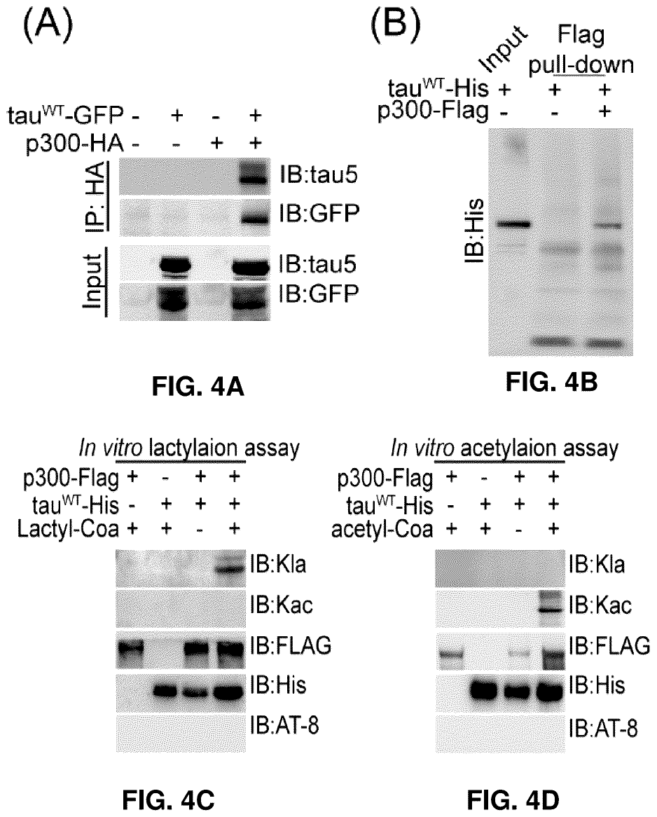

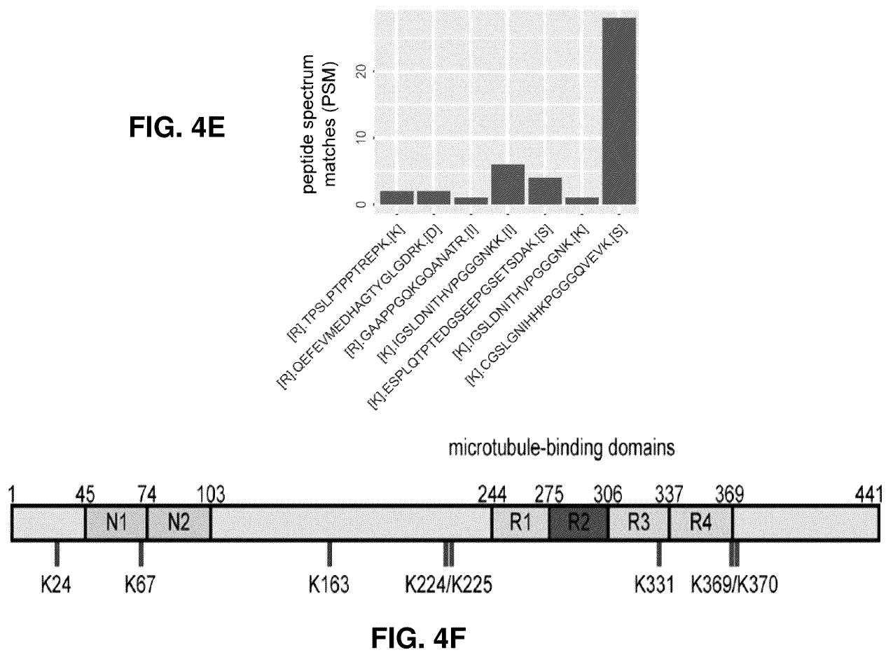

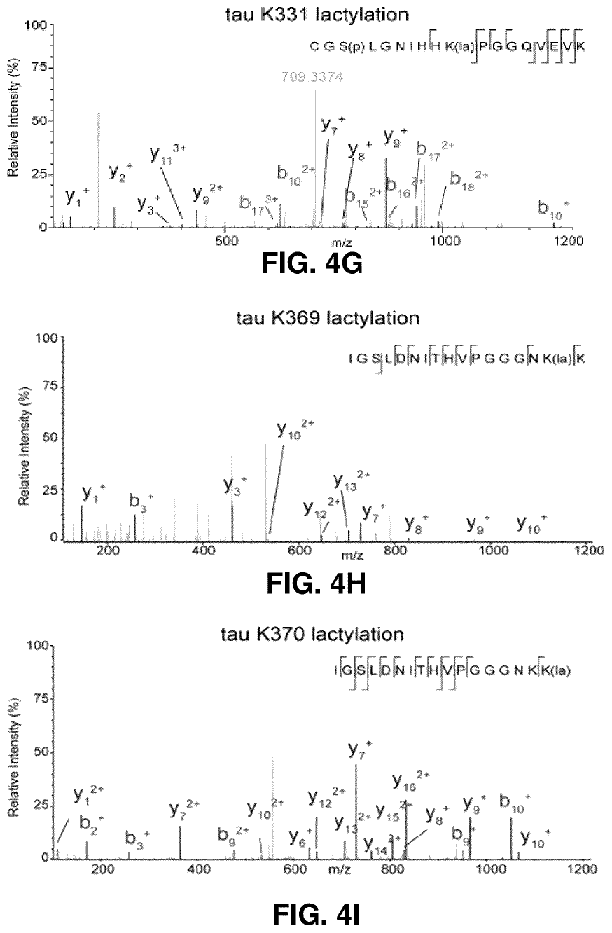

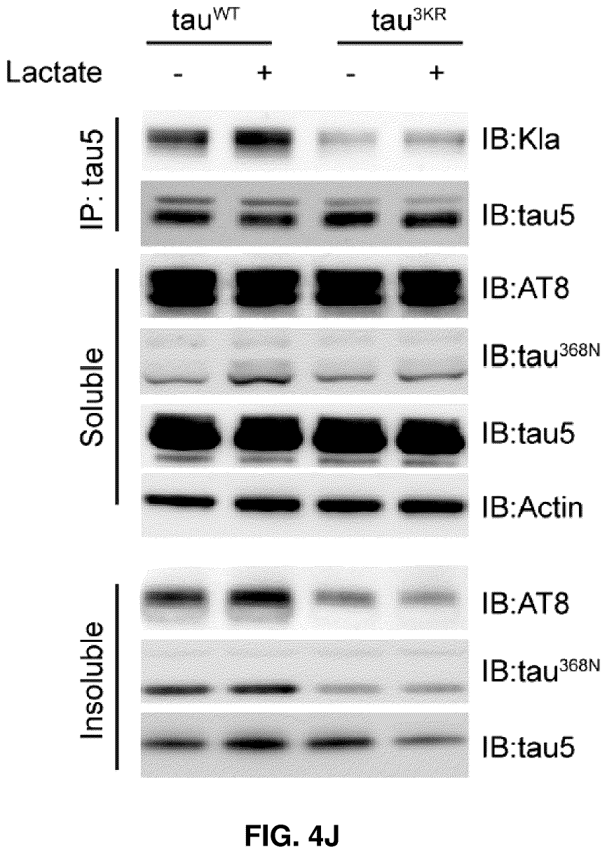

To experimentally examine whether p300 is a mediator of tau lactylation, co-immunoprecipitation was performed in HEK 293T cells transfected with GFP-tagged tau and hemagglutinin (HA)-tagged p300. These data show that tau interacted with p300 (FIG. 4A). Pull-down assay using purified Flag-tagged p300 and his-tagged tau further demonstrated a direct interaction between tau and p300 (FIG. 4B). Notably, AlphaFold predicted that this interaction specifically involves the HAT domain of p300, which has been shown to facilitate the transfer of the lactyl group from lactyl-CoA to tau, and the p300-tau interaction particularly targets the K331-370 residue of tau.

To further confirm that p300 is a lactyl-transferase for tau, purified tau and recombinant p300 proteins were incubated with lactyl-CoA (20 μM), the active form of lactate, in a buffer system designed to facilitate lactylation reactions [5]. These results show that tau lactylation occurred exclusively in the presence of tau, p300, and lactyl-CoA, as detected by the Kla antibody (FIG. 4C). Given that p300 is also an acetyl-transferase, the lysine acetylation (Kac) antibody confirmed the absence of tau acetylation. Additionally, the AT8 antibody shows that tau phosphorylation did not occur, consistent with the lack of kinase that phosphorylates tau in the in vitro system. Reciprocally, when acetyl-CoA was incubated with purified p300 and tau proteins, tau acetylation was induced by the Kac antibody, whereas lactylation was not detected by the Kla antibody (FIG. 4D).

To identify the lactylation sites of the in vitro lactylated tau, liquid chromatography tandem mass spectrometry (LC-MS/MS) analysis identified eight lactylated lysine residues in tau, with K331, the most prominent site identified in the human brain upregulated in AD (FIGS. 2A-2F), K369, and potentially K370 showing the highest PSMs (FIG. 4E-4I).

Subsequent mutagenesis studies replacing these lysine residues (K331, K369, and K370) with arginine (tau3KR) profoundly reduced tau lactylation, even under lactate stimulation (FIG. 4J). Concurrently, tau3KR shows reduced level of tau phosphorylation and cleavage in the insoluble fraction (FIG. 4J). These results confirm that p300 mediates tau lactylation.

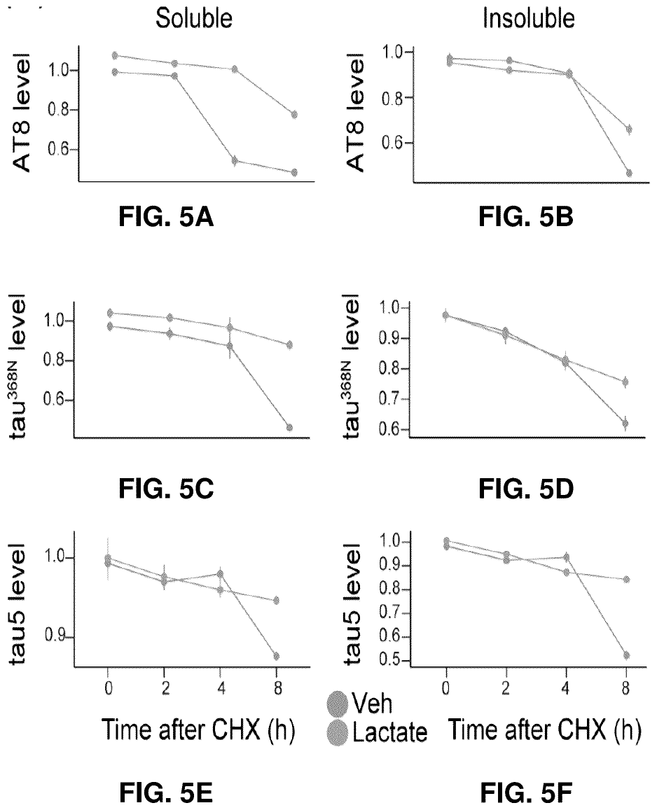

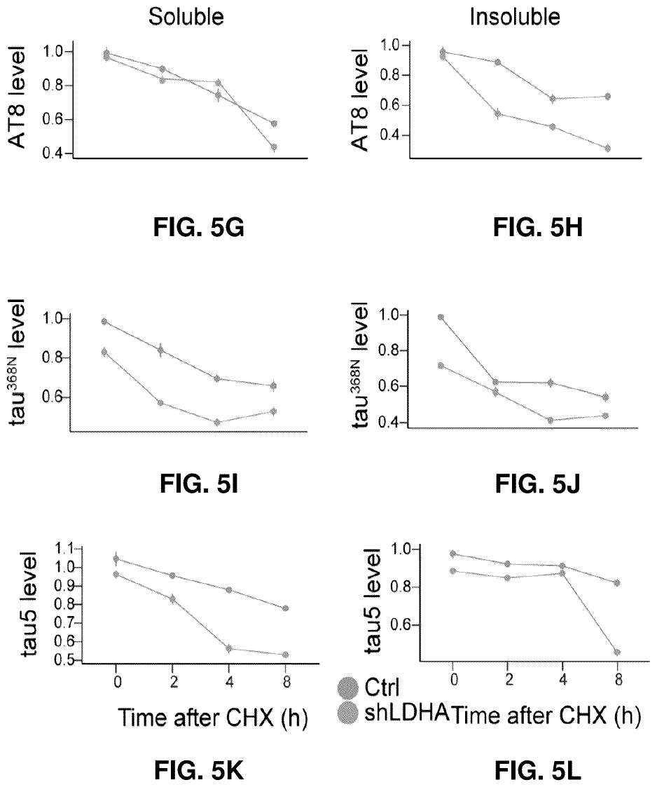

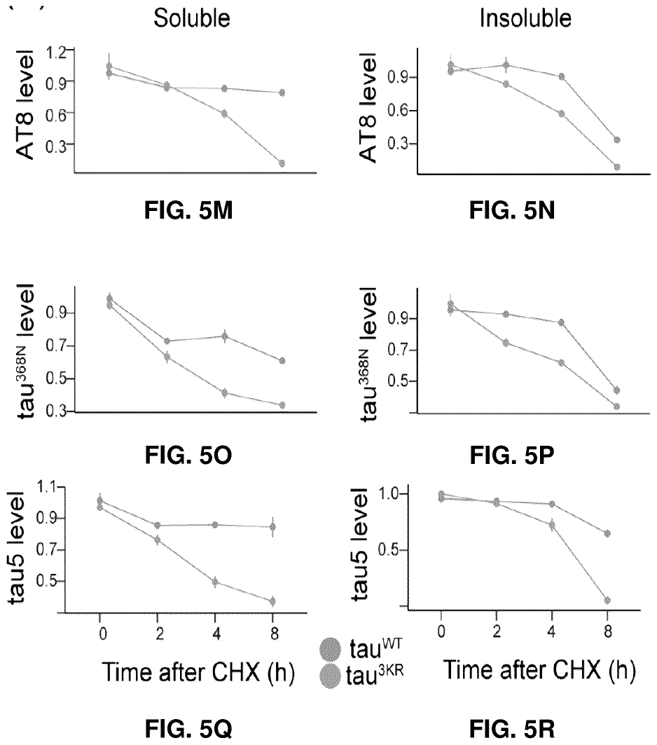

Regulation of turnover and cleavage by lactate-induced tau lactylation To investigate the function of tau lactylation, it was noted that, concurrent with the increase of lactylation of tau, the levels of tau phosphorylation, cleavage, and total tau were also induced by lactate treatment in the insoluble fraction (FIGS. 2A and 2B). Thus, the involvement of lactate-induced tau lactylation in regulating tau turnover was assessed. The turnover rates of wild-type tau were compared with or without lactate treatment, knockdown of LDHA, and tau^{3KR} mutant by treated with cycloheximide (CHX) (FIG. 5A-5R). Lactate treatment resulted in an increased half-life of phosphorylated tau, cleaved tau, and total tau (FIG. 5A-5F). Conversely, knockdown of LDHA (FIG. 5G-5L) or tau^{3KR} mutant (FIG. 5M-5R) resulted in a decreased half-life of phosphorylated tau, cleaved tau, and total tau.

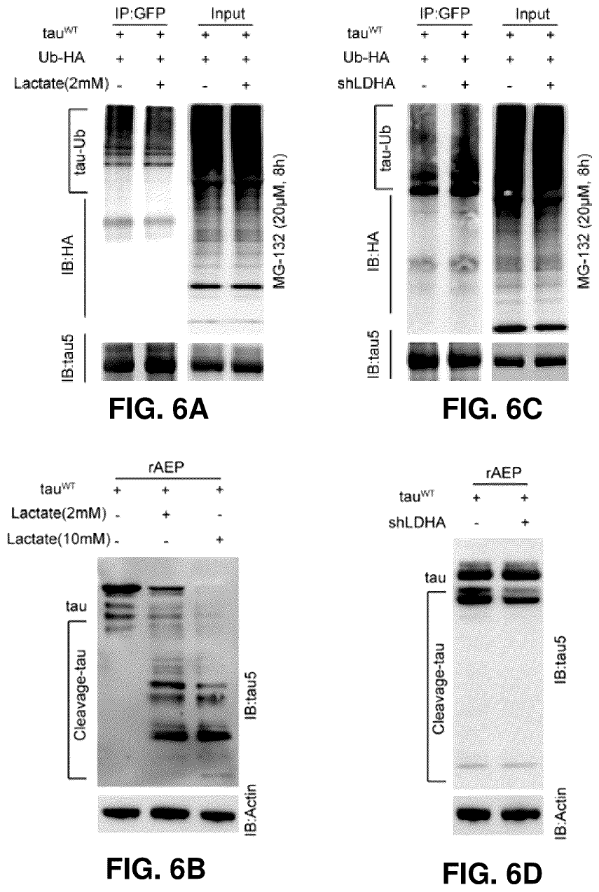

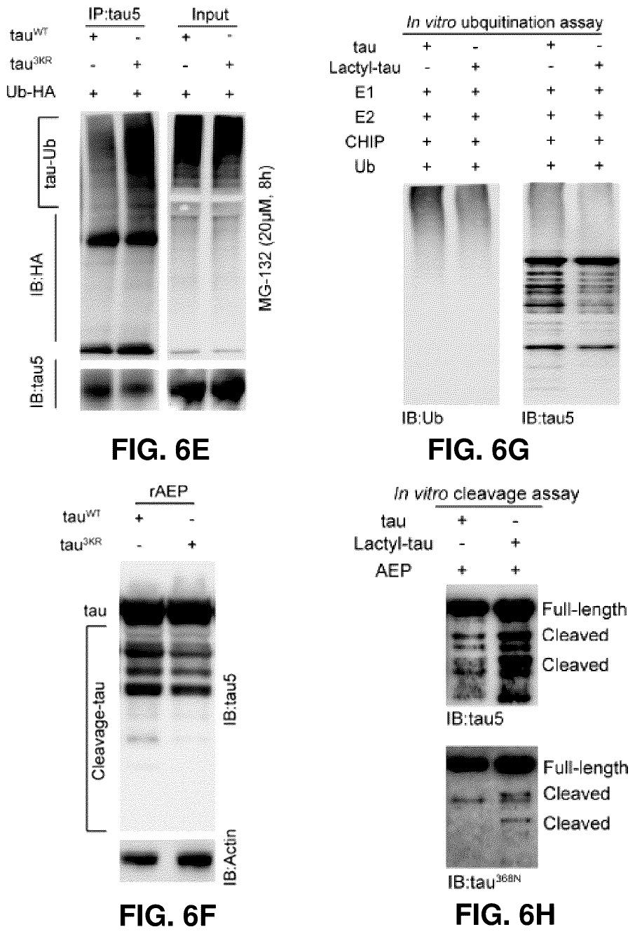

Ubiquitination of tau marks tau for degradation, typically through the proteasome pathway. To directly test whether lactylation inhibits tau ubiquitination, HEK 293T cells were transfected with expression plasmids encoding GFP-tagged wild-type tau-383 and HA-tagged ubiquitin. Cells were then treated with lactate to induce lactylation and MG132, a proteasome inhibitor, to block the proteasome-mediated degradation. After tau was immunoprecipitated by using an anti-GFP antibody, its ubiquitination was detected by using an anti-HA antibody. The data show that lactate inhibited polyubiquitination of tau (FIG. 6A). In contrast, knockdown of LDHA or tau^{3KR} mutant, which reduced tau lactylation (FIGS. 3K-3S and 4J), enhanced tau ubiquitination (FIGS. 6C and 6E).

Additionally, when incubating cell lysate with recombinant 6-secretase (rAEP), the data show that lactate treatment enhanced AEP-induced tau cleavage in a lactate dose-dependent manner (FIG. 6B). Conversely, knockdown of LDHA or tau^{3KR} mutant reduced AEP-induced tau cleavage in cells. (FIGS. 6D and 6F).

Finally, it was examined whether lactylation of tau directly inhibits its ubiquitination and enhances it cleavage. Control and in vitro lactylated tau (lactyl-tau) were incubated with ubiquitin complex proteins in an in vitro ubiquitination assay buffer. The data show that lactyl-tau underwent less ubiquitination (FIG. 6G). Moreover, by using the in vitro tau cleavage assay, the data show that lactyl-tau underwent increased cleavage (FIG. 6H). Together, these data suggest roles of lactylation as an important PTM that regulates tau turnover, ubiquitination, and cleavage.

### Discussion

These findings reveal a significant role of lysine lactylation in tau associated with AD. Elevated lactylation of tau in AD brain samples were detected by proteomics, particularly at the lysine residue K331. The observed upregulation of lactate signature genes and lactylation of tau correlates with increased tau phosphorylation and cleavage, highlighting lactate as an important mediator of tau pathophysiology. These results suggest that lactylation not only serves as a biomarker for AD but also potentially contributes to tau's pathogenic features, such as its aggregation and impaired clearance. Furthermore, the in vitro experiments indicate that lactate directly induces tau lactylation and affects its turnover, providing new insights into the metabolic regulation of tau and offering potential therapeutic avenues for targeting lactate metabolism in AD.

While the analysis of the published quantitative and qualitative proteomics dataset that include 47 AD and 38 control human brain samples [18] revealed that lysine 331 (K331) in tau is a prominent site (FIGS. 2A-2F). Interestingly, this study shows that lysine 331 (K331) in tau shows the highest PSM score in the in vitro lactylation assay (FIGS. 4A-4J). K331 is a particularly dynamic site that can undergo multiple types of post-translational modifications (PTMs), each with distinct implications for tau's function and pathology [9]. This lysine residue can be modified by ubiquitination, acetylation, methylation, and sumoylation, among others. The ability of K331 to undergo such diverse modifications underscores its important role in modulating tau's behavior and highlights the complex regulatory mechanisms that govern the involvement of tau in AD and other neurodegenerative diseases involving tauopathies. Understanding the specific conditions under which each modification occurs and their regulatory effects could provide valuable insights into therapeutic strategies aimed at mitigating tau pathology.

In the postmortem human brain samples, certain lactylation sites-K311, K321, and potentially K317—are detected exclusively in the AD but not in control samples. Since these sites were not identified in p300-mediated in vitro lactylated tau, this suggests the possibility that other lactyltransferases other than p300 may be involved in mediating tau lactylation at these ectopic sites in AD.

Additionally, all lactylation sites identified in human samples (FIGS. 2A-2F) are within the microtubule-binding domains of tau, where positively charged proline-rich regions interact with the negatively charged microtubule surface. This suggests that tau lactylation might regulate microtubule binding, and in AD, aberrant or increased tau lactylation leads to tau dysfunction.

While studies have shown that lactate levels increase during early mild cognitive impairment (MCI) but decrease in later stages to control levels, these findings reveal a progressive buildup of lactylated tau at K331 in the brain based on Braak stages (FIGS. 2A-2F). This suggests that the irreversible accumulation of lactylated tau in the insoluble fraction may contribute to disease progression. The accumulation at lactylated tau K331, a residue within tau's microtubule-binding domain, could interfere with tau's normal role in stabilizing microtubules, leading to impaired neuronal function. Unlike the dynamic fluctuations in lactate levels, the buildup of lactylated tau appears to persist, potentially serving as a pathological marker for the transition from MCI to more severe neurodegenerative stages. This could indicate that lactylation at K331 triggers or exacerbates tau misfolding and aggregation, forming insoluble tau species that further contribute to NFT formation and neuronal toxicity in AD.

In the brain, lactate primarily originates from astrocytic glycolysis, where glucose is either directly converted to lactate to serve as a key energy reserve or transferred to neurons to facilitate metabolism and ATP production. This study shows that lactate dehydrogenase A (LDHA) plays an important role in supplying lactate for tau lactylation (FIGS. 2A-2F). Analysis of the published scRNA-seq dataset revealed that upregulation of LDHA gene expression occurred specifically in excitatory neurons.

While these results show that tau lactylation is elevated in AD, tau lactylation is also detected in the control brain (FIGS. 1A-1J and FIGS. 2A-2F), suggesting that this tau PTM plays a physiological role in neuronal function. Indeed, the role of lactate for brain remains much of debate and what is clear is that lactate is required for physiological function but meanwhile when dysregulated contribute to disease progression. In a recent study, the authors show that restoration of glucose metabolism by inhibition of indoleamine-2,3-dioxygenase 1 (IDO1), an enzyme that metabolizes tryptophan to kynurenine (KYN), improves lactate production in AD mouse models and rescue hippocampal memory function [28].

### Example 2: Lactylation of K281 Occurs in Tauopathology

To test whether the Tau-K281 lactylation occurs in tauopathology, immunostaining with a custom antibody against Tau-Lys281 was carried out in PS19 tauopathy mice (carrying the expression of P301S pathogenic mutant tau) at 9 months of age, when PS19 mice showed tauopathy in the hippocampus.

The polyclonal antibody was generated by immunization with a synthetic peptide including a lactylated lysine residue corresponding to a tau peptide sequence, conjugated to a carrier protein to enhance immunogenicity. The peptide included the lactylated lysine modification at the target position, and the resulting antiserum was affinity-purified to enrich for antibodies recognizing lactylated tau.

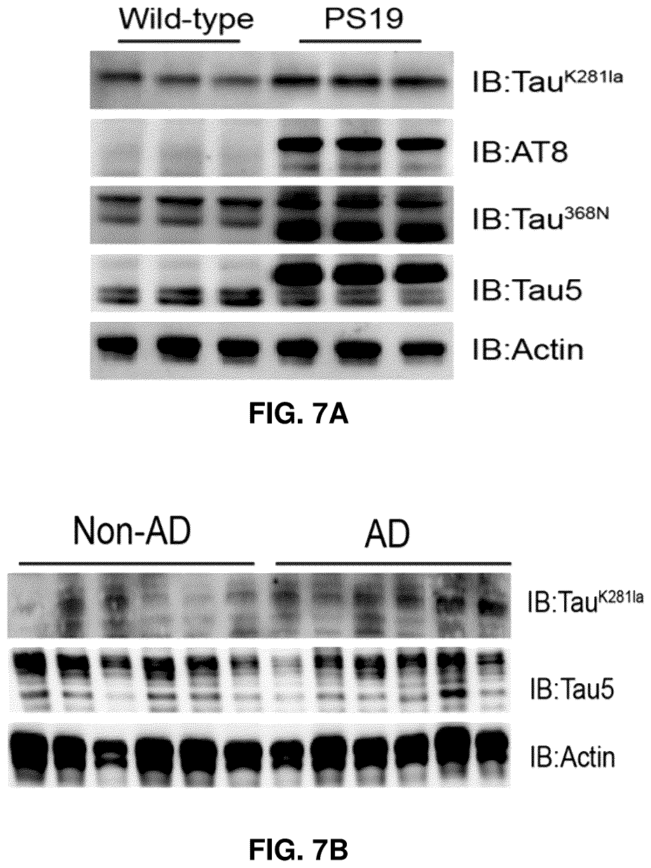

PS19 mice showed significantly more immunoreactivity throughout the hippocampus than control mice, indicating elevated Tau-K281 lactylation (FIG. 7A). In wild-type mice hippocampus, immunostaining for Tau-Lys281 lactylation area only week punctate signals in CA1 pyramidal neurons and the dentate gyrus (DG) (FIG. 7A). By contrast, PS19 mice showed a significantly increase in Tau-Lys281 lactylation, with widespread labeling of neurons across CA1 and the DG (FIG. 7A). Similarly, pan-lactyl lysine immunoreactivity was not detected in wild-type brains but significantly up-regulated in PS19 hippocampus, with strong cytoplasmic and nuclear staining observed in CA1 pyramidal cells and through into the DG granule cell layer (FIG. 7A). AT8 (phospho-tau) immunoreactivity was minimal showed in wild-type tissue, whereas PS19 mice showed extensive AT8-positive tau aggregates that co-localized with regions of elevated lactylation (FIG. 7A). Collectively, these data demonstrate that both tau-specific lactylation and global protein lactylation are significantly increased in the PS19 tauopathy mouse model, and that the spatial distribution of elevated lactylation closely parallels tau phosphorylation pathology. These findings support the concept that lactylation may operate downstream of metabolic dysregulation and upstream of tau aggregation in vivo, as previously implicated in human Alzheimer's disease.

To further confirm the immunohistochemical findings, Western blot analyses of hippocampal lysates from wild-type and PS19 mice was performed. Consistent with the elevated Tau-Lys281 lactylation observed in IHC, immunoblotting with the Tau-Lys281 lactylation antibody showed markedly increased tau lactylation at site 281 in PS19 hippocampus, whereas wild-type lysates displayed only minimal signal (FIG. 7A). In parallel, AT8 immunoblotting revealed robust phospho-tau accumulation in PS19 mice hippocampus, in contrast to the weak or barely detectable AT8 signal in wild-type hippocampus (FIG. 7A). Immunoblotting for Tau368N and Tau5 demonstrated substantially higher levels of transgenic tau in PS19 hippocampus, as expected for the P301S tau overexpression model (FIG. 7A). All the western blot results used actin as a loading control and confirmed equivalent protein amounts across samples.

To determine whether Tau-K281 lactylation has also happened in human Alzheimer's disease, postmortem cortical and hippocampal tissue from AD patients and age-matched non-AD controls were examined. Immunohistochemistry using the Tau-Lys281 lactylation antibody revealed low to minimal TauK281la signal in non-AD cortex and hippocampus, with only weakly positive neurons (FIG. 7B). In contrast, AD brains showed a significantly increase in TauK281 lactylation, characterized by widespread neurons labeling throughout the cortex, as well as strong staining in the hippocampal granule cell layer (FIG. 7B). The distribution of increased TauK281la immunoreactivity corresponded closely to regions known to exhibit abundant tau pathology in AD.

To further confirm these histological observations, western blotting of prefrontal brain lysates from non-AD and AD cases was performed (FIG. 7B). Consistent with the IHC results, AD samples showed markedly elevated TauK281 lactylation, whereas non-AD lysates displayed only weak or barely detectable signal (FIG. 7B). Total tau levels detected by Tau5 were higher in AD tissue, reflecting the accumulation of pathological tau species in disease, while actin confirmed equal protein loading across samples (FIG. 7B).

### Example 3: Lactylation Influences Assembly of Pathogenic Tau

### Methods

To test whether specific lactylation site (K281-lac) tau peptide can influence the ability to disassemble pathogenic tau, two lactylated peptide variants at K281 (Peptide A, Peptide B, and a non-lactylated K281 peptide control) were evaluated for their ability to reduce Tau fibril extracted from PS19 and control mouse brain. The amino acid sequences of the peptides A and B including a lactyllysine at position 281 of Tau 441 (K281la) are as follows:

(SEQ ID NO: 7; Tau K281la peptide A:
276-286)
CQIINK (K-lactyl) LDLSN;
and
(SEQ ID NO: 8; Tau K281la peptide B:
279-290)
CINK (K-lactyl) LDLSNVQSKC.

The corresponding (control) peptide that does not include a lactylation of lysine at position 281 of Tau 441 (K281) is as follows: CQIINKKLDLSN (SEQ ID NO:9; Tau K281la unmodified peptide: 279-290).

Tau fibril disassembly was quantified after 48 h of incubation using dot blot analysis with the specific antibody GT38, which selectively recognizes Tau fibril.

### Results

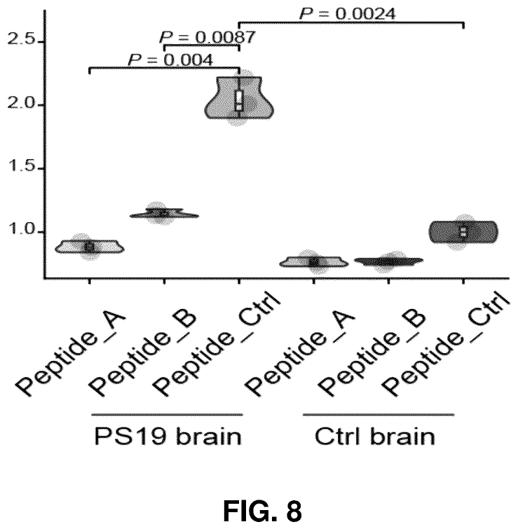

In PS19 brains, Peptide A reduced GT38 signal relative to control mice and the control peptide, indicating robust disassembly activity. Peptide B produced a moderate but reproducible reduction in GT38 immunoreactivity, suggesting partial activity. In contrast, control peptide showed no effect (see FIG. 8).

Together, these data demonstrate that lactylated K281 peptide sequence result in disassembling pathological tau formation in PS19 brain extracts. The data indicate that a tau peptide including a defined lactylation site (e.g., K281-lac) may reduce tau fibril formation and therefore holds potential therapeutic utility.

### REFERENCES

[1] Batra R, Arnold M, Worheide M A, Allen M, Wang X, Blach C, et al. The landscape of metabolic brain alterations in Alzheimer's disease. Alzheimers Dement. 2022.
[2] Vander Heiden M G, Cantley L C, Thompson C B. Understanding the Warburg effect: the metabolic requirements of cell proliferation. Science. 2009; 324:1029-33.
[3] Traxler L, Herdy J R, Stefanoni D, Eichhorner S, Pelucchi S, Szucs A, et al. Warburg-like metabolic transformation underlies neuronal degeneration in sporadic Alzheimer's disease. Cell Metab. 2022; 34:1248-63 e6.
[4] Yang C, Pan R Y, Guan F, Yuan Z. Lactate metabolism in neurodegenerative diseases. Neural Regen Res. 2024; 19:69-74.
[5] Zhang D, Tang Z, Huang H, Zhou G, Cui C, Weng Y, et al. Metabolic regulation of gene expression by histone lactylation. Nature. 2019; 574:575-80.
[6] Pan R Y, He L, Zhang J, Liu X, Liao Y, Gao J, et al. Positive feedback regulation of microglial glucose metabolism by histone H4 lysine 12 lactylation in Alzheimer's disease. Cell Metab. 2022; 34:634-48 e6.
[7] Liu X, Zhang Y, Li W, Zhou X. Lactylation, an emerging hallmark of metabolic reprogramming: Current progress and open challenges. Front Cell Dev Biol. 2022; 10:972020.
[8] Wang M Y, Zhou Y, Li W L, Zhu L Q, Liu D. Friend or foe: Lactate in neurodegenerative diseases. Ageing Res Rev. 2024; 101:102452.
[9] Alquezar C, Arya S, Kao A W. Tau Post-translational Modifications: Dynamic Transformers of Tau Function, Degradation, and Aggregation. Front Neurol. 2020; 11:595532.
[10] Hoover B R, Reed M N, Su J, Penrod R D, Kotilinek L A, Grant M K, et al. Tau mislocalization to dendritic spines mediates synaptic dysfunction independently of neurodegeneration. Neuron. 2010; 68:1067-81.
[11] Askew E B, Bai S, Blackwelder A J, Wilson E M. Transcriptional synergy between melanoma antigen gene protein-All (MAGE-11) and p300 in androgen receptor signaling. J Biol Chem. 2010; 285:21824-36.
[12] Kamitani T, Kito K, Nguyen H P, Yeh E T. Characterization of NEDD8, a developmentally down-regulated ubiquitin-like protein. J Biol Chem. 1997; 272:28557-62.
[13] Wegmann S, Bennett R E, Delorme L, Robbins A B, Hu M, McKenzie D, et al. Experimental evidence for the age dependence of tau protein spread in the brain. Sci Adv. 2019; 5:eaaw6404.
[14] Orsburn B C. Proteome Discoverer-A Community Enhanced Data Processing Suite for Protein Informatics. Proteomes. 2021; 9.
[15] Morabito S, Miyoshi E, Michael N, Shahin S, Martini A C, Head E, et al. Single-nucleus chromatin accessibility and transcriptomic characterization of Alzheimer's disease. Nat Genet. 2021; 53:1143-55.
[16] Higginbotham L, Ping L, Dammer E B, Duong D M, Zhou M, Gearing M, et al. Integrated proteomics reveals brain-based cerebrospinal fluid biomarkers in asymptomatic and symptomatic Alzheimer's disease. Sci Adv. 2020; 6.
[17] Lu Z, Zheng X, Shi M, Yin Y, Liang Y, Zou Z, et al. Lactylation: The emerging frontier in post-translational modification. Front Genet. 2024; 15:1423213.
[18] Wesseling H, Mair W, Kumar M, Schlaffner C N, Tang S, Beerepoot P, et al. Tau PTM Profiles Identify Patient Heterogeneity and Stages of Alzheimer's Disease. Cell. 2020; 183:1699-713 e13.
[19] Yan Y, Wang X, Chaput D, Shin M K, Koh Y, Gan L, et al. X-linked ubiquitin-specific peptidase 11 increases tauopathy vulnerability in women. Cell. 2022; 185:3913-30 e19.
[20] Shelton L B, Baker J D, Zheng D, Sullivan L E, Solanki P K, Webster J M, et al. Hsp90 activator Aha1 drives production of pathological tau aggregates. Proc Natl Acad Sci USA. 2017; 114:9707-12.
[21] Frame A K, Sinka J L, Courchesne M, Muhammad R A, Grahovac-Nemeth S, Bernards M A, et al. Altered neuronal lactate dehydrogenase A expression affects cognition in a sex- and age-dependent manner. iScience. 2024; 27:110342.
[22] Butterfield D A, Halliwell B. Oxidative stress, dysfunctional glucose metabolism and Alzheimer disease. Nat Rev Neurosci. 2019; 20:148-60.
[23] Brooks G A. Lactate. Sports Medicine. 2007; 37:341-3.
[24] Jha M K, Lee I K, Suk K. Metabolic reprogramming by the pyruvate dehydrogenase kinase-lactic acid axis: Linking metabolism and diverse neuropathophysiologies. Neurosci Biobehav Rev. 2016; 68:1-19.
[25] March-Diaz R, Lara-Urena N, Romero-Molina C, Heras-Garvin A, Ortega-de San Luis C, Alvarez-Vergara M I, et al. Hypoxia compromises the mitochondrial metabolism of Alzheimer's disease microglia via HIF1. Nat Aging. 2021; 1:385-99.
[26] Moreno-Yruela C, Zhang D, Wei W, Baek M, Liu W, Gao J, et al. Class I histone deacetylases (HDAC1-3) are histone lysine delactylases. Sci Adv. 2022; 8:eabi6696.
[27] Morris G M, Huey R, Olson A J. Using AutoDock for ligand-receptor docking. Curr Protoc Bioinformatics. 2008; Chapter 8:Unit 8 14.
[28] Minhas P S, Jones J R, Latif-Hernandez A, Sugiura Y, Durairaj A S, Wang Q, et al. Restoring hippocampal glucose metabolism rescues cognition across Alzheimer's disease pathologies. Science. 2024; 385:eabm6131.
[29] Stein C, Koch K, Hopfeld J, Lobentanzer S, Lau H, Klein J. Impaired hippocampal and thalamic acetylcholine release in P301L tau-transgenic mice. Brain Res Bull. 2019; 152:134-42.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific forms of the invention described herein. Such equivalents are intended to be encompassed by the following claims.

We claim:

1. A method of detecting or quantifying lactylation of tau protein, comprising detecting lactylated tau protein in a sample comprising tau protein.

2. The method of claim 1, wherein the detecting comprises contacting the sample with one or more binding moieties that selectively bind lactylated tau protein.

3. The method of claim 2, wherein the tau protein is within a biological sample from a subject,
optionally wherein the biological sample comprises cells or a cell lysate or a fraction thereof,
optionally wherein the cells or cell lysate are obtained as a biopsy from the subject.

4. The method of claim 3, wherein the biological sample comprises a bodily fluid selected from the group consisting of Cerebral Spinal Fluid (CSF), blood, plasma, urine, saliva, nasal fluid, bone marrow, brain tissue, vomit and feces.

5. The method of claim 3, wherein the biological sample comprises CSF.

6. The method of claim 3, further comprising determining that the biological sample includes lactylated tau protein if the level of detected binding is higher in the biological sample than in a control.

7. The method of claim 6, wherein the control comprises a recombinant non-lactylated tau protein, or a biological sample comprising a tau protein from a healthy subject.

8. The method of claim 1, wherein binding is detected by a method selected from the group consisting of an immunoassay, immunohistochemistry, Western blotting, surface plasmon resonance, flow cytometry (FACS) analysis, mass spectrometry, chemical labeling and imaging, chromatographic methods, animal behavior based assays, biosensor analyses and biochip analyses.

9. The method of claim 8, wherein the immunoassay is selected from the group consisting of an enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, and immuno-chromatography assays.

10. The method of claim 1, wherein the one or more binding moieties do not bind to non-lactylated tau protein,
optionally wherein the one or more binding moieties binds to lactylated lysine (Kla).

11. The method of claim 1, wherein the one or more binding moieties is selected from the group consisting of a small molecule, a protein, and a nucleic acid.

12. The method of claim 11, wherein the one or more binding moieties comprises an antibody or an antigen binding fragment thereof.

13. The method of claim 12, wherein the antibody or antigen binding fragment thereof comprises an antigen binding domain that immuno-specifically binds to one or more peptide selected form the group consisting of CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIIIH(K-lactyl)PGGGQVEC (SEQ ID NO:10); GNIHH(K-lactyl)PGGGQC (SEQ ID NO:11); CTHVPGGGN(K-lactyl)(K-lactyl)IETHK (SEQ ID NO:13); and CVPGGGN(K-lactyl)(K-lactyl)IETHK (SEQ ID NO: 14).

14. A method of diagnosing a subject with a disease or disorder associated with lactylated tau protein, comprising detecting or quantifying lactylated tau protein within a sample from the subject, wherein the detecting comprises contacting the sample with one or more binding moieties that selectively bind lactylated tau protein,
optionally further comprising diagnosing a subject with a disease or disorder if the level of detected binding is higher in the biological sample than in a control,
optionally wherein the control comprises a recombinant non-lactylated tau protein, or a biological sample comprising a tau protein from a healthy subject or average of a pool from two or more subject.

15. The method of claim 14, wherein the disease or disorder associated with lactylated tau protein is selected from the group consisting of Alzheimer's disease, Argyrophilic grain disease (AGD), Chronic Traumatic Encephalopathy (CTE), Dementia pugilistica (chronic traumatic encephalopathy), frontotemporal dementia, frontotemporal lobar degeneration, gangliocytoma, Ganglioglioma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis,tangle-predominant dementia, lead encephalopathy, tuberous sclerosis, Hallervorden-Spatz disease, lipofuscinosis, and corticobasal degeneration.

16. The method of claim 14, wherein the disease or disorder is Alzheimer's disease.

17. The method of claim 14 further comprising treating the subject.

18. The method of claim 17, wherein the treatment comprises a therapy effective for treating diseases and disorders characterized by the presence of lactylated tau protein.

19. The method of claim 17, wherein the treatment comprises administering to the subject an effective amount of a lactylated tau protein or fragment thereof, to reduce or alleviate one or more symptoms of the disease or disorder.

20. The method of claim 16, further comprising administering to the subject an effective amount of a lactylated tau protein or fragment thereof, to reduce or alleviate one or more symptoms of Alzheimer's disease.

21. The method of claim 19, wherein the lactylated tau protein or fragment thereof is administered to the subject at dosage of between 0.01 mg/kg and about 1.0 mg/kg, inclusive.

22. The method of claim 19, wherein the lactylated tau protein or fragment thereof is administered to the subject every day, every two days, every three days, every four days, every five days, every six days, every seven days, once every two weeks, or once a month.

23. The method of claim 19, wherein the lactylated tau protein is selected from the group consisting of CPTPPTREP(K-lactyl)(K-lactyl)VAVVR (SEQ ID NO:4); PPTREP(K-lactyl)(K-lactyl)VAVVRC (SEQ ID NO:5); CQIINK(K-lactyl)LDLSN (SEQ ID NO:7); CINK(K-lactyl)LDLSNVQSKC (SEQ ID NO:8); GNIHH(K-lactyl)PGGGQVEC (SEQ ID NO:10); GNIHH(K-lactyl)PGGGQC (SEQ ID NO: 11); CTHVPGGGN(K-lactyl)(K-lactyl)IETHK (SEQ ID NO:13); and CVPGGGN(K-lactyl)(K-lactyl)IETHK (SEQ ID NO:14).

24. The method of claim 19, wherein the lactylated tau protein is selected from the group consisting of CQIINK(K-lactyl)LDLSN (SEQ ID NO:7) and CINK(K-lactyl)LDLSNVQSKC (SEQ ID NO:8).

25. The method of claim 19, wherein the lactylated tau protein is CQIINK(K-lactyl)LDLSN (SEQ ID NO:7).
